SEQUENCE LISTING (257 sequences)

SEQ ID NO: 1 - protein, 5 aa, Artificial Sequence
  Description of Artificial Sequence Synthetic peptide
NYNMH

SEQ ID NO: 2 - protein, 17 aa, Artificial Sequence
  Description of Artificial Sequence Synthetic peptide
TIYPGNDDTSYNQKFKD

SEQ ID NO: 3 - protein, 8 aa, Artificial Sequence
  Description of Artificial Sequence Synthetic peptide
GGYRAMDY

SEQ ID NO: 4 - protein, 16 aa, Artificial Sequence
  Description of Artificial Sequence Synthetic peptide
RSSQSIVYSNGNTYLG

SEQ ID NO: 5 - protein, 7 aa, Artificial Sequence
  Description of Artificial Sequence Synthetic peptide
KVSNRFS

SEQ ID NO: 6 - protein, 9 aa, Artificial Sequence
  Description of Artificial Sequence Synthetic peptide
FQGSHVPYT

SEQ ID NO: 7 - protein, 5 aa, Artificial Sequence
  Description of Artificial Sequence Synthetic peptide
DYYIN

SEQ ID NO: 8 - protein, 17 aa, Artificial Sequence
  Description of Artificial Sequence Synthetic peptide
RIYPGIGNTYYNKKFKG

SEQ ID NO: 9 - protein, 9 aa, Artificial Sequence
  Description of Artificial Sequence Synthetic peptide
GHYGRGMDY

SEQ ID NO: 10 - protein, 17 aa, Artificial Sequence
  Description of Artificial Sequence Synthetic peptide
KSSQSLLNSIDQKNYLA

SEQ ID NO: 11 - protein, 7 aa, Artificial Sequence
  Description of Artificial Sequence Synthetic peptide
FASTKES

SEQ ID NO: 12 - protein, 9 aa, Artificial Sequence
  Description of Artificial Sequence Synthetic peptide
QQHYSTPWT

SEQ ID NO: 13 - protein, 5 aa, Artificial Sequence
  Description of Artificial Sequence Synthetic peptide
RAWMN

SEQ ID NO: 14 - protein, 19 aa, Artificial Sequence
  Description of Artificial Sequence Synthetic peptide
RIKRKTDGETTDYAAPVKG

SEQ ID NO: 15 - protein, 7 aa, Artificial Sequence
  Description of Artificial Sequence Synthetic peptide
SNRAFDI

SEQ ID NO: 16 - protein, 17 aa, Artificial Sequence
  Description of Artificial Sequence Synthetic peptide
KSSQSVLYAGNNRNYLA

SEQ ID NO: 17 - protein, 7 aa, Artificial Sequence
  Description of Artificial Sequence Synthetic peptide
QASTRAS

SEQ ID NO: 18 - protein, 9 aa, Artificial Sequence
  Description of Artificial Sequence Synthetic peptide
QQYYTPPLA

SEQ ID NO: 19 - protein, 6 aa, Artificial Sequence
  Description of Artificial Sequence Synthetic peptide
SYYWSW

SEQ ID NO: 20 - protein, 16 aa, Artificial Sequence
  Description of Artificial Sequence Synthetic peptide
YIYYSGSTNYNPSLKS

SEQ ID NO: 21 - protein, 7 aa, Artificial Sequence
  Description of Artificial Sequence Synthetic peptide
GKTGSAA

SEQ ID NO: 22 - protein, 11 aa, Artificial Sequence
  Description of Artificial Sequence Synthetic peptide
RASQGISRWLA

SEQ ID NO: 23 - protein, 7 aa, Artificial Sequence
  Description of Artificial Sequence Synthetic peptide
AASSLQS

SEQ ID NO: 24 - protein, 9 aa, Artificial Sequence
  Description of Artificial Sequence Synthetic peptide
QQTVSFPIT

SEQ ID NO: 25 - protein, 8 aa, Artificial Sequence
  Description of Artificial Sequence Synthetic peptide
GYTFTNYN

SEQ ID NO: 26 - protein, 8 aa, Artificial Sequence
  Description of Artificial Sequence Synthetic peptide
IYPGNDDT

SEQ ID NO: 27 - protein, 10 aa, Artificial Sequence
  Description of Artificial Sequence Synthetic peptide
ARGGYRAMDY

SEQ ID NO: 28 - protein, 11 aa, Artificial Sequence
  Description of Artificial Sequence Synthetic peptide
QSIVYSNGNTY

SEQ ID NO: 29 - protein, 3 aa, Artificial Sequence
  Description of Artificial Sequence Synthetic peptide
KVS

SEQ ID NO: 30 - protein, 8 aa, Artificial Sequence
  Description of Artificial Sequence Synthetic peptide
GYSFTDYY

SEQ ID NO: 31 - protein, 8 aa, Artificial Sequence
  Description of Artificial Sequence Synthetic peptide
IYPGIGNT

SEQ ID NO: 32 - protein, 11 aa, Artificial Sequence
  Description of Artificial Sequence Synthetic peptide
ARGHYGRGMDY

SEQ ID NO: 33 - protein, 12 aa, Artificial Sequence
  Description of Artificial Sequence Synthetic peptide
QSLLNSIDQKNY

SEQ ID NO: 34 - protein, 3 aa, Artificial Sequence
  Description of Artificial Sequence Synthetic peptide
FAS

SEQ ID NO: 35 - protein, 8 aa, Artificial Sequence
  Description of Artificial Sequence Synthetic peptide
GLTFERAW

SEQ ID NO: 36 - protein, 10 aa, Artificial Sequence
  Description of Artificial Sequence Synthetic peptide
IKRKTDGETT

SEQ ID NO: 37 - protein, 9 aa, Artificial Sequence
  Description of Artificial Sequence Synthetic peptide
AGSNRAFDI

SEQ ID NO: 38 - protein, 12 aa, Artificial Sequence
  Description of Artificial Sequence Synthetic peptide
QSVLYAGNNRNY

SEQ ID NO: 39 - protein, 3 aa, Artificial Sequence
  Description of Artificial Sequence Synthetic peptide
QAS

SEQ ID NO: 40 - protein, 8 aa, Artificial Sequence
  Description of Artificial Sequence Synthetic peptide
GGSISSYY

SEQ ID NO: 41 - protein, 7 aa, Artificial Sequence
  Description of Artificial Sequence Synthetic peptide
IYYSGST

SEQ ID NO: 42 - protein, 9 aa, Artificial Sequence
  Description of Artificial Sequence Synthetic peptide
ARGKTGSAA

SEQ ID NO: 43 - protein, 6 aa, Artificial Sequence
  Description of Artificial Sequence Synthetic peptide
QGISRW

SEQ ID NO: 44 - protein, 3 aa, Artificial Sequence
  Description of Artificial Sequence Synthetic peptide
AAS

SEQ ID NO: 45 - protein, 7 aa, Artificial Sequence
  Description of Artificial Sequence Synthetic peptide
GYTFTNY

SEQ ID NO: 46 - protein, 4 aa, Artificial Sequence
  Description of Artificial Sequence Synthetic peptide
PGND

SEQ ID NO: 47 - protein, 6 aa, Artificial Sequence
  Description of Artificial Sequence Synthetic peptide
GYRAMD

SEQ ID NO: 48 - protein, 12 aa, Artificial Sequence
  Description of Artificial Sequence Synthetic peptide
SQSIVYSNGNTY

SEQ ID NO: 49 - protein, 6 aa, Artificial Sequence
  Description of Artificial Sequence Synthetic peptide
GSHVPY

SEQ ID NO: 50 - protein, 7 aa, Artificial Sequence
  Description of Artificial Sequence Synthetic peptide
GYSFTDY

SEQ ID NO: 51 - protein, 4 aa, Artificial Sequence
  Description of Artificial Sequence Synthetic peptide
PGIG

SEQ ID NO: 52 - protein, 7 aa, Artificial Sequence
  Description of Artificial Sequence Synthetic peptide
HYGRGMD

SEQ ID NO: 53 - protein, 13 aa, Artificial Sequence
  Description of Artificial Sequence Synthetic peptide
SQSLLNSIDQKNY

SEQ ID NO: 54 - protein, 6 aa, Artificial Sequence
  Description of Artificial Sequence Synthetic peptide
HYSTPW

SEQ ID NO: 55 - protein, 7 aa, Artificial Sequence
  Description of Artificial Sequence Synthetic peptide
GLTFERA

SEQ ID NO: 56 - protein, 6 aa, Artificial Sequence
  Description of Artificial Sequence Synthetic peptide
RKTDGE

SEQ ID NO: 57 - protein, 5 aa, Artificial Sequence
  Description of Artificial Sequence Synthetic peptide
NRAFD

SEQ ID NO: 58 - protein, 13 aa, Artificial Sequence
  Description of Artificial Sequence Synthetic peptide
SQSVLYAGNNRNY

SEQ ID NO: 59 - protein, 6 aa, Artificial Sequence
  Description of Artificial Sequence Synthetic peptide
YYTPPL

SEQ ID NO: 60 - protein, 7 aa, Artificial Sequence
  Description of Artificial Sequence Synthetic peptide
GGSISSY

SEQ ID NO: 61 - protein, 3 aa, Artificial Sequence
  Description of Artificial Sequence Synthetic peptide
YSG

SEQ ID NO: 62 - protein, 5 aa, Artificial Sequence
  Description of Artificial Sequence Synthetic peptide
KTGSA

SEQ ID NO: 63 - protein, 7 aa, Artificial Sequence
  Description of Artificial Sequence Synthetic peptide
SQGISRW

SEQ ID NO: 64 - protein, 6 aa, Artificial Sequence
  Description of Artificial Sequence Synthetic peptide
TVSFPI

SEQ ID NO: 65 - protein, 10 aa, Artificial Sequence
  Description of Artificial Sequence Synthetic peptide
ASGYTFTNYN

SEQ ID NO: 66 - protein, 17 aa, Artificial Sequence
  Description of Artificial Sequence Synthetic peptide
IYPGNDDTSYNQKFKDR

SEQ ID NO: 67 - protein, 7 aa, Artificial Sequence
  Description of Artificial Sequence Synthetic peptide
GGYRAMD

SEQ ID NO: 68 - protein, 13 aa, Artificial Sequence
  Description of Artificial Sequence Synthetic peptide
SSQSIVYSNGNTY

SEQ ID NO: 69 - protein, 12 aa, Artificial Sequence
  Description of Artificial Sequence Synthetic peptide
KVSNRFSGVPDR

SEQ ID NO: 70 - protein, 10 aa, Artificial Sequence
  Description of Artificial Sequence Synthetic peptide
ASGYSFTDYY

SEQ ID NO: 71 - protein, 17 aa, Artificial Sequence
  Description of Artificial Sequence Synthetic peptide
IYPGIGNTYYNKKFKGR

SEQ ID NO: 72 - protein, 8 aa, Artificial Sequence
  Description of Artificial Sequence Synthetic peptide
GHYGRGMD

SEQ ID NO: 73 - protein, 14 aa, Artificial Sequence
  Description of Artificial Sequence Synthetic peptide
SSQSLLNSIDQKNY

SEQ ID NO: 74 - protein, 12 aa, Artificial Sequence
  Description of Artificial Sequence Synthetic peptide
FASTKESGVPDR

SEQ ID NO: 75 - protein, 10 aa, Artificial Sequence
  Description of Artificial Sequence Synthetic peptide
ASGLTFERAW

SEQ ID NO: 76 - protein, 19 aa, Artificial Sequence
  Description of Artificial Sequence Synthetic peptide
IKRKTDGETTDYAAPVKGR

SEQ ID NO: 77 - protein, 6 aa, Artificial Sequence
  Description of Artificial Sequence Synthetic peptide
SNRAFD

SEQ ID NO: 78 - protein, 14 aa, Artificial Sequence
  Description of Artificial Sequence Synthetic peptide
SSQSVLYAGNNRNY

SEQ ID NO: 79 - protein, 12 aa, Artificial Sequence
  Description of Artificial Sequence Synthetic peptide
QASTRASGVPDR

SEQ ID NO: 80 - protein, 10 aa, Artificial Sequence
  Description of Artificial Sequence Synthetic peptide
VSGGSISSYY

SEQ ID NO: 81 - protein, 16 aa, Artificial Sequence
  Description of Artificial Sequence Synthetic peptide
IYYSGSTNYNPSLKSR

SEQ ID NO: 82 - protein, 6 aa, Artificial Sequence
  Description of Artificial Sequence Synthetic peptide
GKTGSA

SEQ ID NO: 83 - protein, 8 aa, Artificial Sequence
  Description of Artificial Sequence Synthetic peptide
ASQGISRW

SEQ ID NO: 84 - protein, 12 aa, Artificial Sequence
  Description of Artificial Sequence Synthetic peptide
AASSLQSGVPSR

SEQ ID NO: 85 - protein, 117 aa, Artificial Sequence
  Description of Artificial Sequence Synthetic polypeptide
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 86 - protein, 112 aa, Artificial Sequence
  Description of Artificial Sequence Synthetic polypeptide
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 87 - protein, 118 aa, Artificial Sequence
  Description of Artificial Sequence Synthetic polypeptide
QVQLVQSGAEVKKPGASVKVSCKASGYSFTDYYINWVRQAPGQGLEWMGRIYPGIGNTYYNKKFKGRVTITRDTSASTAYMELSSLRSEDTAVYYCARGHYGRGMDYWGQGTLVTVSS

SEQ ID NO: 88 - protein, 113 aa, Artificial Sequence
  Description of Artificial Sequence Synthetic polypeptide
DIVMTQSPDSLAVSLGERATINCKSSQSLLNSIDQKNYLAWYQQKPGQPPKLLIYFASTKESGVPDRFSGSGSGTDFTLTISGLQAEDVAVYFCQQHYSTPWTFGGGTKVEIR

SEQ ID NO: 89 - protein, 118 aa, Artificial Sequence
  Description of Artificial Sequence Synthetic polypeptide
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSS

SEQ ID NO: 90 - protein, 113 aa, Artificial Sequence
  Description of Artificial Sequence Synthetic polypeptide
DIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIK

SEQ ID NO: 91 - protein, 115 aa, Artificial Sequence
  Description of Artificial Sequence Synthetic polypeptide
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARGKTGSAAWGQGTLVTVSS

SEQ ID NO: 92 - protein, 107 aa, Artificial Sequence
  Description of Artificial Sequence Synthetic polypeptide
DIQMTQSPSSVSASVGDRVTITCRASQGISRWLAWYQQKPGKAPKLLIYAASSLQSGVPSRFSGSGSGTDFTLTISSLQPEDFATYYCQQTVSFPITFGGGTKVEIK

SEQ ID NO: 93 - protein, 5 aa, Artificial Sequence
  Description of Artificial Sequence Synthetic peptide
SYWIT

SEQ ID NO: 94 - protein, 17 aa, Artificial Sequence
  Description of Artificial Sequence Synthetic peptide
DIYPGSGSTNHIEKFKS

SEQ ID NO: 95 - protein, 11 aa, Artificial Sequence
  Description of Artificial Sequence Synthetic peptide
GYGSSYGYFDY

SEQ ID NO: 96 - protein, 11 aa, Artificial Sequence
  Description of Artificial Sequence Synthetic peptide
RASENIYSYLA

SEQ ID NO: 97 - protein, 7 aa, Artificial Sequence
  Description of Artificial Sequence Synthetic peptide
TAKTLAE

SEQ ID NO: 98 - protein, 9 aa, Artificial Sequence
  Description of Artificial Sequence Synthetic peptide
QHQYGPPFT

SEQ ID NO: 99 - protein, 5 aa, Artificial Sequence
  Description of Artificial Sequence Synthetic peptide
SYWMH

SEQ ID NO: 100 - protein, 17 aa, Artificial Sequence
  Description of Artificial Sequence Synthetic peptide
NIDPSDSDTHYNQKFKD

SEQ ID NO: 101 - protein, 10 aa, Artificial Sequence
  Description of Artificial Sequence Synthetic peptide
GYSKYYAMDY

SEQ ID NO: 102 - protein, 16 aa, Artificial Sequence
  Description of Artificial Sequence Synthetic peptide
RSSQSIVHSYGNTYLE

SEQ ID NO: 103 - protein, 7 aa, Artificial Sequence
  Description of Artificial Sequence Synthetic peptide
KVSNRFS

SEQ ID NO: 104 - protein, 9 aa, Artificial Sequence
  Description of Artificial Sequence Synthetic peptide
FQGSHVPYT

SEQ ID NO: 105 - protein, 11 aa, Artificial Sequence
  Description of Artificial Sequence Synthetic peptide
YGNYGENAMDY

SEQ ID NO: 106 - protein, 9 aa, Artificial Sequence
  Description of Artificial Sequence Synthetic peptide
FQGSHVPFT

SEQ ID NO: 107 - protein, 5 aa, Artificial Sequence
  Description of Artificial Sequence Synthetic peptide
DYYIH

SEQ ID NO: 108 - protein, 17 aa, Artificial Sequence
  Description of Artificial Sequence Synthetic peptide
RIDPEDGETKYAPKFQG

SEQ ID NO: 109 - protein, 5 aa, Artificial Sequence
  Description of Artificial Sequence Synthetic peptide
GGFAY

SEQ ID NO: 110 - protein, 11 aa, Artificial Sequence
  Description of Artificial Sequence Synthetic peptide
ASSSVSSSYLY

SEQ ID NO: 111 - protein, 7 aa, Artificial Sequence
  Description of Artificial Sequence Synthetic peptide
STSNLAS

SEQ ID NO: 112 - protein, 9 aa, Artificial Sequence
  Description of Artificial Sequence Synthetic peptide
HQWSSHPYT

SEQ ID NO: 113 - protein, 5 aa, Artificial Sequence
  Description of Artificial Sequence Synthetic peptide
SYWVH

SEQ ID NO: 114 - protein, 17 aa, Artificial Sequence
  Description of Artificial Sequence Synthetic peptide
NIDPSDSDTHYSPSFQG

SEQ ID NO: 115 - protein, 11 aa, Artificial Sequence
  Description of Artificial Sequence Synthetic peptide
GGTGTLAYFAY

SEQ ID NO: 116 - protein, 16 aa, Artificial Sequence
  Description of Artificial Sequence Synthetic peptide
RSSQSLVHSYGNTYLY

SEQ ID NO: 117 - protein, 7 aa, Artificial Sequence
  Description of Artificial Sequence Synthetic peptide
RVSNRFS

SEQ ID NO: 118 - protein, 9 aa, Artificial Sequence
  Description of Artificial Sequence Synthetic peptide
FQGTHVPYT

SEQ ID NO: 119 - protein, 5 aa, Artificial Sequence
  Description of Artificial Sequence Synthetic peptide
GYGIS

SEQ ID NO: 120 - protein, 17 aa, Artificial Sequence
  Description of Artificial Sequence Synthetic peptide
WISAYGGETNYAQKLQG

SEQ ID NO: 121 - protein, 11 aa, Artificial Sequence
  Description of Artificial Sequence Synthetic peptide
EAGSSWYDFDL

SEQ ID NO: 122 - protein, 11 aa, Artificial Sequence
  Description of Artificial Sequence Synthetic peptide
RASQGISSWLA

SEQ ID NO: 123 - protein, 7 aa, Artificial Sequence
  Description of Artificial Sequence Synthetic peptide
AASNLQS

SEQ ID NO: 124 - protein, 9 aa, Artificial Sequence
  Description of Artificial Sequence Synthetic peptide
QQGASFPIT

SEQ ID NO: 125 - protein, 8 aa, Artificial Sequence
  Description of Artificial Sequence Synthetic peptide
GYTFTSYW

SEQ ID NO: 126 - protein, 8 aa, Artificial Sequence
  Description of Artificial Sequence Synthetic peptide
IYPGSGST

SEQ ID NO: 127 - protein, 13 aa, Artificial Sequence
  Description of Artificial Sequence Synthetic peptide
ATGYGSSYGYFDY

SEQ ID NO: 128 - protein, 6 aa, Artificial Sequence
  Description of Artificial Sequence Synthetic peptide
ENIYSY

SEQ ID NO: 129 - protein, 3 aa, Artificial Sequence
  Description of Artificial Sequence Synthetic peptide
TAK

SEQ ID NO: 130 - protein, 8 aa, Artificial Sequence
  Description of Artificial Sequence Synthetic peptide
IDPSDSDT

SEQ ID NO: 131 - protein, 12 aa, Artificial Sequence
  Description of Artificial Sequence Synthetic peptide
ARGYSKYYAMDY

SEQ ID NO: 132 - protein, 11 aa, Artificial Sequence
  Description of Artificial Sequence Synthetic peptide
QSIVHSYGNTY

SEQ ID NO: 133 - protein, 13 aa, Artificial Sequence
  Description of Artificial Sequence Synthetic peptide
ASYGNYGENAMDY

SEQ ID NO: 134 - protein, 8 aa, Artificial Sequence
  Description of Artificial Sequence Synthetic peptide
GFNIKDYY

SEQ ID NO: 135 - protein, 8 aa, Artificial Sequence
  Description of Artificial Sequence Synthetic peptide
IDPEDGET

SEQ ID NO: 136 - protein, 7 aa, Artificial Sequence
  Description of Artificial Sequence Synthetic peptide
AKGGFAY

SEQ ID NO: 137 - protein, 7 aa, Artificial Sequence
  Description of Artificial Sequence Synthetic peptide
SSVSSSY

SEQ ID NO: 138 - protein, 3 aa, Artificial Sequence
  Description of Artificial Sequence Synthetic peptide
STS

SEQ ID NO: 139 - protein, 8 aa, Artificial Sequence
  Description of Artificial Sequence Synthetic peptide
GYSFTSYW

SEQ ID NO: 140 - protein, 13 aa, Artificial Sequence
  Description of Artificial Sequence Synthetic peptide
VRGGTGTLAYFAY

SEQ ID NO: 141 - protein, 11 aa, Artificial Sequence
  Description of Artificial Sequence Synthetic peptide
QSLVHSYGNTY

SEQ ID NO: 142 - protein, 3 aa, Artificial Sequence
  Description of Artificial Sequence Synthetic peptide
RVS

SEQ ID NO: 143 - protein, 8 aa, Artificial Sequence
  Description of Artificial Sequence Synthetic peptide
GYTFRGYG

SEQ ID NO: 144 - protein, 8 aa, Artificial Sequence
  Description of Artificial Sequence Synthetic peptide
ISAYGGET

SEQ ID NO: 145 - protein, 13 aa, Artificial Sequence
  Description of Artificial Sequence Synthetic peptide
AREAGSSWYDFDL

SEQ ID NO: 146 - protein, 6 aa, Artificial Sequence
  Description of Artificial Sequence Synthetic peptide
QGISSW

SEQ ID NO: 147 - protein, 7 aa, Artificial Sequence
  Description of Artificial Sequence Synthetic peptide
GYTFTSY

SEQ ID NO: 148 - protein, 4 aa, Artificial Sequence
  Description of Artificial Sequence Synthetic peptide
PGSG

SEQ ID NO: 149 - protein, 9 aa, Artificial Sequence
  Description of Artificial Sequence Synthetic peptide
YGSSYGYFD

SEQ ID NO: 150 - protein, 7 aa, Artificial Sequence
  Description of Artificial Sequence Synthetic peptide
SENIYSY

SEQ ID NO: 151 - protein, 6 aa, Artificial Sequence
  Description of Artificial Sequence Synthetic peptide
QYGPPF

SEQ ID NO: 152 - protein, 4 aa, Artificial Sequence
  Description of Artificial Sequence Synthetic peptide
PSDS

SEQ ID NO: 153 - protein, 8 aa, Artificial Sequence
  Description of Artificial Sequence Synthetic peptide
YSKYYAMD

SEQ ID NO: 154 - protein, 12 aa, Artificial Sequence
  Description of Artificial Sequence Synthetic peptide
SQSIVHSYGNTY

SEQ ID NO: 155 - protein, 6 aa, Artificial Sequence
  Description of Artificial Sequence Synthetic peptide
GSHVPY

SEQ ID NO: 156 - protein, 9 aa, Artificial Sequence
  Description of Artificial Sequence Synthetic peptide
GNYGENAMD

SEQ ID NO: 157 - protein, 6 aa, Artificial Sequence
  Description of Artificial Sequence Synthetic peptide
GSHVPF

SEQ ID NO: 158 - protein, 7 aa, Artificial Sequence
  Description of Artificial Sequence Synthetic peptide
GFNIKDY

SEQ ID NO: 159 - protein, 4 aa, Artificial Sequence
  Description of Artificial Sequence Synthetic peptide
PEDG

SEQ ID NO: 160 - protein, 3 aa, Artificial Sequence
  Description of Artificial Sequence Synthetic peptide
GFA

SEQ ID NO: 161 - protein, 8 aa, Artificial Sequence
  Description of Artificial Sequence Synthetic peptide
SSSVSSSY

SEQ ID NO: 162 - protein, 6 aa, Artificial Sequence
  Description of Artificial Sequence Synthetic peptide
WSSHPY

SEQ ID NO: 163 - protein, 7 aa, Artificial Sequence
  Description of Artificial Sequence Synthetic peptide
GYSFTSY

SEQ ID NO: 164 - protein, 9 aa, Artificial Sequence
  Description of Artificial Sequence Synthetic peptide
GTGTLAYFA

SEQ ID NO: 165 - protein, 12 aa, Artificial Sequence
  Description of Artificial Sequence Synthetic peptide
SQSLVHSYGNTY

SEQ ID NO: 166 - protein, 6 aa, Artificial Sequence
  Description of Artificial Sequence Synthetic peptide
GTHVPY

SEQ ID NO: 167 - protein, 7 aa, Artificial Sequence
  Description of Artificial Sequence Synthetic peptide
GYTFRGY

SEQ ID NO: 168 - protein, 4 aa, Artificial Sequence
  Description of Artificial Sequence Synthetic peptide
AYGG

SEQ ID NO: 169 - protein, 9 aa, Artificial Sequence
  Description of Artificial Sequence Synthetic peptide
AGSSWYDFD

SEQ ID NO: 170 - protein, 7 aa, Artificial Sequence
  Description of Artificial Sequence Synthetic peptide
SQGISSW

SEQ ID NO: 171 - protein, 6 aa, Artificial Sequence
  Description of Artificial Sequence Synthetic peptide
GASFPI

SEQ ID NO: 172 - protein, 10 aa, Artificial Sequence
  Description of Artificial Sequence Synthetic peptide
ASGYTFTSYW

SEQ ID NO: 173 - protein, 17 aa, Artificial Sequence
  Description of Artificial Sequence Synthetic peptide
IYPGSGSTNHIEKFKSK

SEQ ID NO: 174 - protein, 10 aa, Artificial Sequence
  Description of Artificial Sequence Synthetic peptide
GYGSSYGYFD

SEQ ID NO: 175 - protein, 8 aa, Artificial Sequence
  Description of Artificial Sequence Synthetic peptide
ASENIYSY

SEQ ID NO: 176 - protein, 12 aa, Artificial Sequence
  Description of Artificial Sequence Synthetic peptide
TAKTLAEGVPSR

SEQ ID NO: 177 - protein, 17 aa, Artificial Sequence
  Description of Artificial Sequence Synthetic peptide
IDPSDSDTHYNQKFKDR

SEQ ID NO: 178 - protein, 9 aa, Artificial Sequence
  Description of Artificial Sequence Synthetic peptide
GYSKYYAMD

SEQ ID NO: 179 - protein, 13 aa, Artificial Sequence
  Description of Artificial Sequence Synthetic peptide
SSQSIVHSYGNTY

SEQ ID NO: 180 - protein, 12 aa, Artificial Sequence
  Description of Artificial Sequence Synthetic peptide
KVSNRFSGVPDR

SEQ ID NO: 181 - protein, 17 aa, Artificial Sequence
  Description of Artificial Sequence Synthetic peptide
IDPSDSDTHYNQKFKDK

SEQ ID NO: 182 - protein, 10 aa, Artificial Sequence
  Description of Artificial Sequence Synthetic peptide
YGNYGENAMD

SEQ ID NO: 183 - protein, 10 aa, Artificial Sequence
  Description of Artificial Sequence Synthetic peptide
ASGFNIKDYY

SEQ ID NO: 184 - protein, 17 aa, Artificial Sequence
  Description of Artificial Sequence Synthetic peptide
IDPEDGETKYAPKFQGK

SEQ ID NO: 185 - protein, 4 aa, Artificial Sequence
  Description of Artificial Sequence Synthetic peptide
GGFA

SEQ ID NO: 186 - protein, 9 aa, Artificial Sequence
  Description of Artificial Sequence Synthetic peptide
ASSSVSSSY

SEQ ID NO: 187 - protein, 12 aa, Artificial Sequence
  Description of Artificial Sequence Synthetic peptide
STSNLASGVPAR

SEQ ID NO: 188 - protein, 10 aa, Artificial Sequence
  Description of Artificial Sequence Synthetic peptide
ASGYSFTSYW

SEQ ID NO: 189 - protein, 17 aa, Artificial Sequence
  Description of Artificial Sequence Synthetic peptide
IDPSDSDTHYSPSFQGH

SEQ ID NO: 190 - protein, 10 aa, Artificial Sequence
  Description of Artificial Sequence Synthetic peptide
GGTGTLAYFA

SEQ ID NO: 191 - protein, 13 aa, Artificial Sequence
  Description of Artificial Sequence Synthetic peptide
SSQSLVHSYGNTY

SEQ ID NO: 192 - protein, 12 aa, Artificial Sequence
  Description of Artificial Sequence Synthetic peptide
RVSNRFSGVPDR

SEQ ID NO: 193 - protein, 10 aa, Artificial Sequence
  Description of Artificial Sequence Synthetic peptide
ASGYTFRGYG

SEQ ID NO: 194 - protein, 17 aa, Artificial Sequence
  Description of Artificial Sequence Synthetic peptide
ISAYGGETNYAQKLQGR

SEQ ID NO: 195 - protein, 10 aa, Artificial Sequence
  Description of Artificial Sequence Synthetic peptide
EAGSSWYDFD

SEQ ID NO: 196 - protein, 8 aa, Artificial Sequence
  Description of Artificial Sequence Synthetic peptide
ASQGISSW

SEQ ID NO: 197 - protein, 12 aa, Artificial Sequence
  Description of Artificial Sequence Synthetic peptide
AASNLQSGVPSR

SEQ ID NO: 198 - protein, 120 aa, Artificial Sequence
  Description of Artificial Sequence Synthetic polypeptide
QVQLVQSGAEVKKPGASVKVSCKASGYTFTSYWITWVKQAPGQGLEWIGDIYPGSGSTNHIEKFKSKATLTVDTSISTAYMELSRLRSDDTAVYYCATGYGSSYGYFDYWGQGTLVTVSS

SEQ ID NO: 199 - protein, 107 aa, Artificial Sequence
  Description of Artificial Sequence Synthetic polypeptide
DIQMTQSPSSLSASVGDRVTITCRASENIYSYLAWYQQKPGKAPKLLIYTAKTLAEGVPSRFSGSGSGTDFTLTISSLQPEDFATYYCQHQYGPPFTFGQGTKLEIK

SEQ ID NO: 200 - protein, 119 aa, Artificial Sequence
  Description of Artificial Sequence Synthetic polypeptide
QVQLVQSGAEVKKPGASVKVSCKASGYTFTSYWMHWVRQAPGQGLEWMGNIDPSDSDTHYNQKFKDRVTMTRDTSTSTVYMELSSLRSEDTAVYYCARGYSKYYAMDYWGQGTLVTVSS

SEQ ID NO: 201 - protein, 112 aa, Artificial Sequence
  Description of Artificial Sequence Synthetic polypeptide
DIVMTQTPLSLSVTPGQPASISCRSSQSIVHSYGNTYLEWYLQKPGQSPQLLIYKVSNRFSGVPDRFSGSGSGTDFTLKISRVEAEDVGVYYCFQGSHVPYTFGQGTKLEIK

SEQ ID NO: 202 - protein, 120 aa, Artificial Sequence
  Description of Artificial Sequence Synthetic polypeptide
QVKLQESGAELVRPGSSVKLSCKASGYTFTSYWMHWVKQRPIQGLEWIGNIDPSDSDTHYNQKFKDKATLTVDNSSSTAYMQLSSLTSEDSAVYYCASYGNYGENAMDYWGQGTSVTVSS

SEQ ID NO: 203 - protein, 112 aa, Artificial Sequence
  Description of Artificial Sequence Synthetic polypeptide
DILMTQTPLSLPVSLGDQASISCRSSQSIVHSYGNTYLEWYLQKPGQSPKLLIYKVSNRFSGVPDRFSGSGSGTDFTLKISRVEAEDLGVYYCFQGSHVPFTFGSGTKLEIK

SEQ ID NO: 204 - protein, 114 aa, Artificial Sequence
  Description of Artificial Sequence Synthetic polypeptide
EVQLQQSGAELVKPGASVKLSCTASGFNIKDYYIHWVKQRTEQGLEWIGRIDPEDGETKYAPKFQGKATITADTSSNTAYLQLNSLTSEDTAVYSCAKGGFAYWGQGTLVTVSA

SEQ ID NO: 205 - protein, 108 aa, Artificial Sequence
  Description of Artificial Sequence Synthetic polypeptide
QIVLTQSPAIMSASPGEKVTLTCSASSSVSSSYLYWYQQKPGSSPKLWIYSTSNLASGVPARFSGSGSGTSYSLTISSMEAEDAASYFCHQWSSHPYTFGGGTKLEIK

SEQ ID NO: 206 - protein, 120 aa, Artificial Sequence
  Description of Artificial Sequence Synthetic polypeptide
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTLAYFAYWGQGTLVTVSS

SEQ ID NO: 207 - protein, 112 aa, Artificial Sequence
  Description of Artificial Sequence Synthetic polypeptide
DVVMTQSPLSLPVTLGQPASISCRSSQSLVHSYGNTYLYWFQQRPGQSPRLLIYRVSNRFSGVPDRFSGSGSGTDFTLKISRVEAEDVGVYYCFQGTHVPYTFGGGTKVEIK

SEQ ID NO: 208 - protein, 120 aa, Artificial Sequence
  Description of Artificial Sequence Synthetic polypeptide
QVQLVQSGAEVKKPGASVKVSCKASGYTFRGYGISWVRQAPGQGLEWMGWISAYGGETNYAQKLQGRVTMTTDTSTSTAYMELRSLRSDDTAVYYCAREAGSSWYDFDLWGRGTLVTVSS

SEQ ID NO: 209 - protein, 107 aa, Artificial Sequence
  Description of Artificial Sequence Synthetic polypeptide
DIQMTQSPSSVSASVGDRVTITCRASQGISSWLAWYQQKPGKAPKLLIYAASNLQSGVPSRFSGSGSGTDFTLTISSLQPEDFATYYCQQGASFPITFGGGTKVEIK

SEQ ID NO: 210 - protein, 5 aa, Artificial Sequence
  Description of Artificial Sequence Synthetic peptide
SYWMN

SEQ ID NO: 211 - protein, 17 aa, Artificial Sequence
  Description of Artificial Sequence Synthetic peptide
MIDPSDSETHNAQKFQG

SEQ ID NO: 212 - protein, 8 aa, Artificial Sequence
  Description of Artificial Sequence Synthetic peptide
LYRWYFDV

SEQ ID NO: 213 - protein, 11 aa, Artificial Sequence
  Description of Artificial Sequence Synthetic peptide
RASEIVGTYVS

SEQ ID NO: 214 - protein, 7 aa, Artificial Sequence
  Description of Artificial Sequence Synthetic peptide
GASNRYT

SEQ ID NO: 215 - protein, 9 aa, Artificial Sequence
  Description of Artificial Sequence Synthetic peptide
GQSYNFPYT

SEQ ID NO: 216 - protein, 5 aa, Artificial Sequence
  Description of Artificial Sequence Synthetic peptide
SYYMH

SEQ ID NO: 217 - protein, 17 aa, Artificial Sequence
  Description of Artificial Sequence Synthetic peptide
IINPSGGSTSYAQKFQG

SEQ ID NO: 218 - protein, 10 aa, Artificial Sequence
  Description of Artificial Sequence Synthetic peptide
STLWFSEFDY

SEQ ID NO: 219 - protein, 14 aa, Artificial Sequence
  Description of Artificial Sequence Synthetic peptide
SGTSSDVGGHNYVS

SEQ ID NO: 220 - protein, 7 aa, Artificial Sequence
  Description of Artificial Sequence Synthetic peptide
DVTKRPS

SEQ ID NO: 221 - protein, 9 aa, Artificial Sequence
  Description of Artificial Sequence Synthetic peptide
LSYAGSRVY

SEQ ID NO: 222 - protein, 8 aa, Artificial Sequence
  Description of Artificial Sequence Synthetic peptide
GYTFTSYW

SEQ ID NO: 223 - protein, 8 aa, Artificial Sequence
  Description of Artificial Sequence Synthetic peptide
IDPSDSET

SEQ ID NO: 224 - protein, 10 aa, Artificial Sequence
  Description of Artificial Sequence Synthetic peptide
ARLYRWYFDV

SEQ ID NO: 225 - protein, 6 aa, Artificial Sequence
  Description of Artificial Sequence Synthetic peptide
EIVGTY

SEQ ID NO: 226 - protein, 3 aa, Artificial Sequence
  Description of Artificial Sequence Synthetic peptide
GAS

SEQ ID NO: 227 - protein, 8 aa, Artificial Sequence
  Description of Artificial Sequence Synthetic peptide
GYTFTSYY

SEQ ID NO: 228 - protein, 8 aa, Artificial Sequence
  Description of Artificial Sequence Synthetic peptide
INPSGGST

SEQ ID NO: 229 - protein, 12 aa, Artificial Sequence
  Description of Artificial Sequence Synthetic peptide
ARSTLWFSEFDY

SEQ ID NO: 230 - protein, 9 aa, Artificial Sequence
  Description of Artificial Sequence Synthetic peptide
SSDVGGHNY

SEQ ID NO: 231 - protein, 3 aa, Artificial Sequence
  Description of Artificial Sequence Synthetic peptide
DVT

SEQ ID NO: 232 - protein, 7 aa, Artificial Sequence
  Description of Artificial Sequence Synthetic peptide
GYTFTSY

SEQ ID NO: 233 - protein, 4 aa, Artificial Sequence
  Description of Artificial Sequence Synthetic peptide
PSDS

SEQ ID NO: 234 - protein, 6 aa, Artificial Sequence
  Description of Artificial Sequence Synthetic peptide
YRWYFD

SEQ ID NO: 235 - protein, 7 aa, Artificial Sequence
  Description of Artificial Sequence Synthetic peptide
SEIVGTY

SEQ ID NO: 236 - protein, 6 aa, Artificial Sequence
  Description of Artificial Sequence Synthetic peptide
SYNFPY

SEQ ID NO: 237 - protein, 4 aa, Artificial Sequence
  Description of Artificial Sequence Synthetic peptide
PSGG

SEQ ID NO: 238 - protein, 8 aa, Artificial Sequence
  Description of Artificial Sequence Synthetic peptide
TLWFSEFD

SEQ ID NO: 239 - protein, 11 aa, Artificial Sequence
  Description of Artificial Sequence Synthetic peptide
GTSSDVGGHNY

SEQ ID NO: 240 - protein, 6 aa, Artificial Sequence
  Description of Artificial Sequence Synthetic peptide
YAGSRV

SEQ ID NO: 241 - protein, 10 aa, Artificial Sequence
  Description of Artificial Sequence Synthetic peptide
ASGYTFTSYW

SEQ ID NO: 242 - protein, 17 aa, Artificial Sequence
  Description of Artificial Sequence Synthetic peptide
IDPSDSETHNAQKFQGK

SEQ ID NO: 243 - protein, 7 aa, Artificial Sequence
  Description of Artificial Sequence Synthetic peptide
LYRWYFD

SEQ ID NO: 244 - protein, 8 aa, Artificial Sequence
  Description of Artificial Sequence Synthetic peptide
ASEIVGTY

SEQ ID NO: 245 - protein, 12 aa, Artificial Sequence
  Description of Artificial Sequence Synthetic peptide
GASNRYTGVPAR

SEQ ID NO: 246 - protein, 6 aa, Artificial Sequence
  Description of Artificial Sequence Synthetic peptide
SYNFPY

SEQ ID NO: 247 - protein, 10 aa, Artificial Sequence
  Description of Artificial Sequence Synthetic peptide
ASGYTFTSYY

SEQ ID NO: 248 - protein, 17 aa, Artificial Sequence
  Description of Artificial Sequence Synthetic peptide
INPSGGSTSYAQKFQGR

SEQ ID NO: 249 - protein, 9 aa, Artificial Sequence
  Description of Artificial Sequence Synthetic peptide
STLWFSEFD

SEQ ID NO: 250 - protein, 12 aa, Artificial Sequence
  Description of Artificial Sequence Synthetic peptide
DVTKRPSGVPDR

SEQ ID NO: 251 - protein, 7 aa, Artificial Sequence
  Description of Artificial Sequence Synthetic peptide
YAGSRVY

SEQ ID NO: 252 - protein, 117 aa, Artificial Sequence
  Description of Artificial Sequence Synthetic polypeptide
QVQLVQSGAEVVKPGASVKLSCKASGYTFTSYWMNWVRQRPGQGLEWIGMIDPSDSETHNAQKFQGKATLTVDKSTSTAYMHLSSLRSEDTAVYYCARLYRWYFDVWGAGTTVTVSS

SEQ ID NO: 253 - protein, 107 aa, Artificial Sequence
  Description of Artificial Sequence Synthetic polypeptide
NIVMTQSPATMSMSPGERVTLSCRASEIVGTYVSWFQQKPGQAPRLLIYGASNRYTGVPARFSGSGSGTDFTLTISSVQPEDLADYHCGQSYNFPYTFGGGTKLEIK

SEQ ID NO: 254 - protein, 119 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARSTLWFSEFDYWGQGTLVTVSS

SEQ ID NO: 255 - protein, 110 aa, Artificial Sequence
  Description of Artificial Sequence Synthetic polypeptide
QSVLTQPSSVSASPGQSITISCSGTSSDVGGHNYVSWYQQHPGKAPKLMIYDVTKRPSGVPDRFSGSKSGNTASLTVSGLQAEDEADYYCLSYAGSRVYVFGTGTKLTVL

SEQ ID NO: 256 - protein, 15 aa, Artificial Sequence
  Description of Artificial Sequence Synthetic peptide
RRCPLYISYDPVCRR

SEQ ID NO: 257 - protein, 19 aa, Artificial Sequence
  Description of Artificial Sequence Synthetic peptide
RRRRCPLYISYDPVCRRRR

(19) United States
(12) Patent Application Publication
(10) Pub. No.: US 2022/0340679 A1
(43) Pub. Date: Oct. 27, 2022

(54) CO-INHIBITION OF CD47/SIRPalpha BINDING AND NEDD8-ACTIVATING ENZYME E1 REGULATORY SUBUNIT FOR THE TREATMENT OF CANCER

(71) Applicant: Gilead Sciences, Inc., Foster City, CA (US)

(72) Inventors: Hikmat H. Assi, San Pablo, CA (US); Mark P. Chao, Palo Alto, CA (US)

(21) Appl. No.: 17/719,122

(22) Filed: Apr. 12, 2022

Related U.S. Application Data
(60) Provisional application No. 63/227,981, filed on Jul. 30, 2021.
(60) Provisional application No. 63/174,971, filed on Apr. 14, 2021.

(51) Int. Cl. C07K 16/28; A61P 35/02; A61K 45/06

(52) U.S. Cl. CPC C07K 16/2896; A61P 35/02; A61K 45/06; C07K 16/2887; A61K 2039/505

(57) ABSTRACT

Provided are methods for treating, mitigating, or preventing or delaying the recurrence or metastasis of, a cancer in a subject comprising co-administering: (a) an agent that inhibits binding between CD47 and SIRPα; and (b) a NEDD8-activating enzyme E1 regulatory subunit (NAE1) inhibitor. Further provided are kits for practicing such methods.

### CROSS-REFERENCE TO RELATED APPLICATIONS

This application claims the benefit under 35 U.S.C. § 119(e) of U.S. Provisional Application No. 63/174,971, filed on Apr. 14, 2021 and U.S. Provisional Application No. 63/227,981, filed on Jul. 30, 2021, which are hereby incorporated herein by reference in their entireties for all purposes.

### SEQUENCE LISTING

The instant application contains a Sequence Listing which has been submitted electronically in ASCII format and is hereby incorporated by reference in its entirety. Said ASCII copy, created on Mar. 3, 2022, is named FSI-008-US-NP_SL.txt and is 81,122 bytes in size.

### BACKGROUND

CD47 is a molecule mediating cancer cell evasion of phagocytosis by the innate immune system. CD47 appears to be an important means by which cancer cells, including cancer stem cells, overcome oftentimes intrinsic expression of their prophagocytic, “eat me,” signals. The progression from normal cell to cancer cell can involve changes in genes and/or gene expression that trigger programmed cell death (PCD) and programmed cell removal (PCR). Many of the steps in cancer progression subvert multiple mechanisms of PCD, and expression of anti-phagocytic signal, CD47, may represent an important checkpoint.

CD47 serves as the ligand for SIRPα, which is expressed on phagocytic cells including macrophages and dendritic cells. When SIRPα is activated by CD47 binding, it initiates a signal transduction cascade resulting in inhibition of phagocytosis. In this way, CD47 functions as an anti-phagocytic signal by delivering a dominant inhibitory signal to phagocytic cells.

CD47 expression is increased on the surface of many cancer cells from a large number of diverse human tumor types including the following primary malignancies, including without limitation hematologic cancers (e.g., leukemias and pre-leukemias) and solid tumor cancers, e.g., head and neck, melanoma, breast, lung, ovarian, pancreatic, colon, bladder, prostate, leiomyosarcoma, glioblastoma, medulloblastoma, oligodendroglioma, glioma, lymphoma, and multiple myeloma. In murine xenograft studies, it has been shown that CD47-blocking antibodies inhibit human cancer growth and metastasis by enabling phagocytosis and elimination of cancer cells from various hematologic malignancies and several solid tumors.

Acute myeloid leukemia (AML) is a common hematological malignancy whose incidence rises from 3:100,000 in young adults to greater than 20:100,000 in older adults. For patients <60 years of age, overall survival (OS) is 40 to 50%, but is only 5% for patients >60 years of age. The majority of newly diagnosed patients with AML are over the age of 60. In this patient population, standard induction chemotherapy is often not an option due to increased treatment-related mortality as a result of age and co-morbidities. Standard of care for AML patients unfit for combination chemotherapy is treatment with hypomethylating agents (e.g., azacitidine, decitabine or guadacitabine) or low dose cytarabine. Despite these frontline treatments, median OS is only about 10 months. In all types of AML, disease relapse is common despite an initial therapeutic response and is the most common reason for death. Standard chemotherapy and allogeneic stem cell transplant (when used) often fail to eradicate all tumor-propagating cells and select for chemotherapy-resistant leukemia-propagating subclones. Patients refractory to salvage therapy are treated palliatively, as current treatment options are extremely limited. These patients have a median survival of 2 months. In addition, patients with newly diagnosed intermediate or higher-risk myelodysplastic syndrome (MDS) and those who relapse after standard care have a poor prognosis and high risk of progression to AML.

Combination therapies using an agent that inhibits binding between CD47 and SIRPα; and a NEDD8-activating enzyme E1 regulatory subunit (NAE1) inhibitor in humans with cancer are of clinical interest, and are provided herein. Additionally, new treatment modalities for relapsed/refractory (R/R) AML and MDS patients, newly diagnosed AML patients ineligible for induction chemotherapy based on age and co-morbidities, and newly diagnosed intermediate/high/very high risk MDS patients are of clinical interest.

### SUMMARY

Provided are methods of treating, mitigating, or preventing or delaying the progression of (e.g., to more aggressive disease), or preventing or delaying the recurrence or metastasis of, a cancer in a subject comprising administering to the subject an effective amount of: (a) an agent that inhibits binding between CD47 and SIRPα; and (b) a NEDD8-activating enzyme E1 regulatory subunit (NAE1) inhibitor. In some embodiments, the agent that inhibits binding between CD47 and SIRPα comprises an antibody that binds to CD47. In some embodiments, the antibody that binds to CD47 is selected from the group consisting of magrolimab, lemzoparlimab, letaplimab, AK117 (ligufalimab), AO-176, IBI-322, ZL-1201, IMC-002, SRF-231, CC-90002 (a.k.a., INBRX-103), NI-1701 (a.k.a., TG-1801) and STI-6643. In some embodiments, the agent that inhibits binding between CD47 and SIRPα comprises an antibody that binds to SIRPα. In some embodiments, the antibody that binds to SIRPα is selected from the group consisting of GS-0189 (a.k.a., FSI-189), CC-95251, BI-765063 and APX-700. In some embodiments, the agent that inhibits binding between CD47 and SIRPα comprises a SIRPα-Fc fusion protein. In some embodiments, the SIRPα-Fc fusion protein is selected from the group consisting of ALX-148, TTI-621, TTI-622, JMT601 (CP0107) and SL-172154. In some embodiments, the NAE1 inhibitor is selected from the group consisting of pevonedistat, TAK-243 and TAS-4464. In some embodiments, the agent that inhibits binding between CD47 and SIRPα and the NAE1 inhibitor are administered concurrently. In some embodiments, the agent that inhibits binding between CD47 and SIRPα and the NAE1 inhibitor are administered sequentially. In some embodiments, the agent that inhibits binding between CD47 and SIRPα; and the NAE1 are administered in a combined synergistic amount. In some embodiments, administration of the agent that inhibits binding between CD47 and SIRPα and the NAE1 inhibitor provides a synergistic effect. In some embodiments, the synergistic effect is increased cancer cell death and/or decreased cancer cell growth when comparing the effect of the combination versus either the agent that inhibits binding between CD47 and SIRPα or the NAE1 inhibitor alone. In some embodiments, the synergistic effect is increased phagocytosis of cancer cells by macrophages when comparing the effect of the combination versus either the agent that inhibits binding between CD47 and SIRPα or the NAE1 inhibitor alone. In some embodiments, the synergistic effect is increased or enhanced cancer cell clearance when comparing the effect of the combination versus either the agent that inhibits binding between CD47 and SIRPα or the NAE1 inhibitor alone.

Further provided are methods of treating, mitigating, or preventing or delaying the progression of (e.g., to more aggressive disease), or preventing or delaying the recurrence or metastasis of, a cancer in a subject comprising administering to the subject an effective amount of: (a) magrolimab; and (b) pevonedistat. In some embodiments, magrolimab and pevonedistat are administered in a combined synergistic amount. In some embodiments, administration of magrolimab and pevonedistat provides a synergistic effect. In some embodiments, the synergistic effect is increased cancer cell death and/or decreased cancer cell growth when comparing the effect of the combination versus either magrolimab or pevonedistat alone. In some embodiments, the synergistic effect is increased phagocytosis of cancer cells by macrophages when comparing the effect of the combination versus either magrolimab or pevonedistat alone. In some embodiments, the synergistic effect is increased or enhanced cancer cell clearance when comparing the effect of the combination versus either magrolimab or pevonedistat alone. In some embodiments, the magrolimab is first administered at a priming dose of less than 10 mg/kg and then administered at one or more therapeutic doses of at least 15 mg/kg, e.g., at least 30 mg/kg, 45 mg/kg, 60 mg/kg. In some embodiments, the magrolimab is administered intravenously, subcutaneously or intratumorally. In some embodiments, the pevonedistat is administered at one or more doses in the range of 10 mg/m² to 50 mg/m². In some embodiments, the pevonedistat is administered orally, intravenously, intramuscularly or subcutaneously.

With respect to embodiments of the methods, in some embodiments, the subject is a human. In some embodiments, the cancer is a hematologic cancer. In some embodiments, the cancer is a solid tumor cancer. In some embodiments, the cancer has increased cell surface expression of CD47. In some embodiments, the solid tumor cancer arises from a primary malignancy selected from the group consisting of: head and neck (HNSCC), melanoma, breast, lung, ovarian, pancreatic, colon, bladder, prostate, leiomyosarcoma, glioblastoma, medulloblastoma, oligodendroglioma, glioma, lymphoma, and multiple myeloma. In some embodiments, the cancer is a leukemia or a pre-leukemia. In some embodiments, the cancer is selected from the group consisting of a myelodysplastic syndrome (MDS), acute myelogenous leukemia (AML), chronic lymphocytic leukemia (CLL), small lymphocytic leukemia (SLL), B-cell acute lymphoblastic leukemia. In some embodiments, the cancer is a lymphoma. In some embodiments, the lymphoma is selected from the group consisting of non-Hodgkin's lymphoma, diffuse large B-cell lymphoma (DLBCL), follicular lymphoma (FL), marginal zone lymphoma, mantle cell lymphoma, Waldenström's macroglobulinemia/lymphoplasmacytic lymphoma, primary mediastinal B-cell lymphoma, Burkitt's lymphoma, B-cell lymphoma unclassified, or post-transplant lymphoproliferative disease (PTLD). In some embodiments, the cancer is relapsed or refractory. In some embodiments, the methods further entail administering a hypomethylation agent. In some embodiments, the hypomethylating agent is selected from azacitidine, decitabine and guadacitabine. In some embodiments, the methods further entail administering an inhibitor of Bcl-2. In some embodiments, the inhibitor of Bcl-2 is selected from the group consisting of venetoclax, obatoclax mesylate, pelcitoclax and navitoclax. In some embodiments, the methods further entail administering one or more therapeutic antibodies. In some embodiments, the therapeutic antibody binds to CD19 (e.g., blinatumomab, tafasitamab, inebilizumab, loncastuximab), CD20 (e.g., rituximab, ofatumumab, obinutuzumab, alemtuzumab, veltuzumab, veltuzumab, ocrelizumab, ocaratuzumab, ublituximab), CD33 (e.g., gemtuzumab, lintuzumab, vadastuximab), CD123 (e.g., talacotuzumab, vibecotamab, flotetuzumab) or hepatitis A virus cellular receptor 2 (HAVCR2; TIM3; CD366) (e.g., sabatolimab, cobolimab).

Further provided are kits. In various embodiments, the kit comprises one or more unitary doses of: (a) an agent that inhibits binding between CD47 and SIRPα; and (b) a NEDD8-activating enzyme E1 regulatory subunit (NAE1) inhibitor. In some embodiments, the agent that inhibits binding between CD47 and SIRPα and the NAE1 inhibitor are in separate containers. In some embodiments, the agent that inhibits binding between CD47 and SIRPα comprises an antibody that binds to CD47. In some embodiments, the antibody that binds to CD47 is selected from the group consisting of magrolimab, lemzoparlimab, letaplimab, AK117 (ligufalimab), AO-176, IBI-322, ZL-1201, IMC-002, SRF-231, CC-90002 (a.k.a., INBRX-103), NI-1701 (a.k.a., TG-1801) and STI-6643. In some embodiments, the agent that inhibits binding between CD47 and SIRPα comprises an antibody that binds to SIRPα. In some embodiments, the antibody that binds to SIRPα is selected from the group consisting of GS-0189 (a.k.a., FSI-189), CC-95251, BI-765063 and APX-700. In some embodiments, the agent that inhibits binding between CD47 and SIRPα comprises a SIRPα-Fc fusion protein. In some embodiments, the SIRPα-Fc fusion protein is selected from the group consisting of ALX-148, TTI-621, TTI-622, JMT601 (CP0107) and SL-172154. In some embodiments, the NAE1 inhibitor is selected from the group consisting of pevonedistat, TAK, 243 and TAS-4464. In some embodiments, the kit comprises one or more unitary doses of magrolimab and one or more unitary doses of pevonedistat. In some embodiments, the kit further comprises one or more unitary doses of a hypomethylation agent. In some embodiments, the hypomethylating agent is selected from azacitidine, decitabine and guadacitabine. In some embodiments, the kit further comprises an inhibitor of Bcl-2. In some embodiments, the inhibitor of Bcl-2 is selected from the group consisting of venetoclax, obatoclax mesylate, pelcitoclax and navitoclax. In some embodiments, the kit further comprises one or more therapeutic antibodies. In some embodiments, the therapeutic antibody binds to CD19 (e.g., blinatumomab, tafasitamab, inebilizumab, loncastuximab), CD20 (e.g., rituximab, ofatumumab, obinutuzumab, alemtuzumab, veltuzumab, veltuzumab, ocrelizumab, ocaratuzumab, ublituximab), CD33 (e.g., gemtuzumab, lintuzumab, vadastuximab), CD123 (e.g., talacotuzumab, vibecotamab, flotetuzumab) or hepatitis A virus cellular receptor 2 (HAVCR2; TIM3; CD366) (e.g., sabatolimab, cobolimab).

### BRIEF DESCRIPTION OF THE DRAWINGS

FIG. 1 illustrates that Nedd8-activating Enzyme Subunit 1 (NAE1) induces cytotoxicity of U937 AML cells in a dose-dependent manner. The growth of U937 cells was monitored in response to increasing concentrations of an NAE1 small molecule inhibitor. Cellular ATP was measured after 72 hours of treatment using a commercial luminescent cell viability assay as a readout for cell number. Growth inhibition was calculated using the following equation: 100×(negative control−test sample/negative control).

FIG. 2 illustrates that NAE1 inhibition enhances in-vitro phagocytosis of U937 AML cells. U937 tumor cells were treated for 24 hours with 1 nM or 100 nM of NAE1 inhibitor, washed, CFSE-labeled, and mixed with M-CSF monocyte-derived macrophages in media containing 10 μg/mL of human IgG4 isotype or anti-CD47 antibody (Magrolimab). After a 2-hour culture period, cells were labeled with a fluorescent anti-CD11b antibody and analyzed on a flow cytometer. Phagocytosis was reported as an index based on the fold increase in the percentage of CD11b+ CFSE+ cells relative to the PBS-treatment. Conditions were tested in triplicate and reported as an average from five different monocyte donors.

FIG. 3 illustrates that NAE1 inhibition in combination with CD47 blockade elicits robust anti-tumor efficacy in an AML xenograft model. Luciferase-expressing U937 tumor cell were transferred to NSG mice by intravenous injection. On day 5, mice were randomized into cohorts and treated with Vehicle [20% (2-Hydroxypropyl)-β-cyclodextrin], azacitidine (7.5 mg/kg QD for 5 days), or NAE1 small molecule inhibitor (120 mg/kg QD for 5 days followed by 2 days/rest) by intraperitoneal injection. Magrolimab was administered 48 hours post chemo treatment (250 μg QD for the entire study) as a single agent or in combination. Tumor growth was monitored using in-vivo bioluminescent imaging and the resulting signal was reported as total flux.

### DETAILED DESCRIPTION

1. Introduction

Provided are methods of treating, mitigating, or preventing or delaying the recurrence or metastasis of, a cancer in a subject by administering: (a) an agent that inhibits binding between CD47 and SIRPα (e.g., magrolimab); and (b) a NEDD8-activating enzyme E1 regulatory subunit (NAE1) inhibitor (e.g., pevonedistat) to the subject. Surprisingly, as demonstrated herein, combined administration of an agent that inhibits binding between a CD47 and SIRPα (e.g., magrolimab) and an NAE1 inhibitor (e.g., pevonedistat) resulted in synergistic (i.e., more than additive) phagocytosis of cancer cells reduction in tumor growth.

2. Therapeutic Agents

a. Agent that Inhibits Binding Between CD47 and SIRPα

i. Antibody or Antigen-Binding Fragment Thereof that Binds to CD47

In various embodiments, the agent that inhibits binding between CD47 and SIRPα CD47 is an antibody or antigen-binding fragment thereof that binds to CD47 (a.k.a., IAP, MER6, OA3; NCBI Gene ID: 961; UniProt Q08722). In various embodiments, an antibody that binds to CD47 has an Fc having effector function. In various embodiments, an antibody that binds to CD47 is an IgG4 or an IgG1. Examples of anti-CD47 antibodies of use include without limitation magrolimab, lemzoparlimab, letaplimab, AK117 (ligufalimab), AO-176, IBI-322, ZL-1201, IMC-002, SRF-231, CC-90002 (a.k.a., INBRX-103), NI-1701 (a.k.a., TG-1801), STI-6643 (Vx-1004), CNTO-7108, RCT-1938, RRx-001, DSP-107, VT-1021 and SGN-CD47M.

In various embodiments, the antibody targeting CD47 is a bi-specific antibody. Examples bi-specific antibodies targeting CD47, include without limitation IBI-322 (CD47/PD-L1), IMM-0306 (CD47/CD20), TJ-L1C4 (CD47/PD-L1), HX-009 (CD47/PD-1), PMC-122 (CD47/PD-L1), PT-217, (CD47/DLL3), IMM-26011 (CD47/FLT3), IMM-0207 (CD47/VEGF), IMM-2902 (CD47/HER2), BH29xx (CD47/PD-L1), IMM-03 (CD47/CD20), IMM-2502 (CD47/PD-L1), HMBD-004B (CD47/BCMA), HMBD-004A (CD47/CD33). Examples of anti-CD47antibodies, such as IBI-188, TJC-4, SHR-1603, HLX-24, LQ-001, IMC-002, ZL-1201, IMM-01, B6H12, GenSci-059, TAY-018, PT-240, 1F8-GMCSF, SY-102 and KD-015.

In various embodiments, the antibody targeting CD47 comprises a VH-CDR1, a VH-CDR2, a VH-CDR3, a VL-CDR1, a VL-CDR2 and a VL-CDR3 comprising the following amino acid sequences (according to Kabat), respectively:
SEQ ID NOs: 1, 2, 3, 4, 5 and 6;
SEQ ID NOs: 7, 8, 9, 10, 11 and 12;
SEQ ID NOs: 13, 14, 15, 16, 17, and 18;
SEQ ID NOs: 19, 20, 21, 22, 23 and 24;
SEQ ID NOs: 210, 211, 212, 213, 214 and 215; or
SEQ ID NOs: 216, 217, 218, 219, 220 and 221.

In various embodiments, the antibody targeting CD47 comprises a VH-CDR1, a VH-CDR2, a VH-CDR3, a VL-CDR1, a VL-CDR2 and a VL-CDR3 comprising the following amino acid sequences (according to IMGT), respectively:
SEQ ID NOs: 25, 26, 27, 28, 29 and 6;
SEQ ID NOs: 30, 31, 32, 33, 34 and 12;
SEQ ID NOs: 35, 36, 37, 38, 39 and 18;
SEQ ID NOs: 40, 41, 42, 43, 44 and 24;
SEQ ID NOs: 222, 223, 224, 225, 226 and 215; or
SEQ ID NOs: 227, 228, 229, 230, 231 and 221.

In various embodiments, the antibody targeting CD47 comprises a VH-CDR1, a VH-CDR2, a VH-CDR3, a VL-CDR1, a VL-CDR2 and a VL-CDR3 comprising the following amino acid sequences (according to Chothia), respectively:
SEQ ID NOs: 45, 46, 47, 48, 29 and 49;
SEQ ID NOs: 50, 51, 52, 53, 34 and 54;
SEQ ID NOs: 55, 56, 57, 58, 39 and 59;
SEQ ID NOs: 60, 61, 62, 62, 44 and 64;
SEQ ID NOs: 232, 233, 234, 235, 226 and 236; or
SEQ ID NOs: 232, 237, 238, 239, 231 and 240.

In various embodiments, the antibody targeting CD47 comprises a VH-CDR1, a VH-CDR2, a VH-CDR3, a VL-CDR1, a VL-CDR2 and a VL-CDR3 comprising the following amino acid sequences (according to Honegger), respectively:
SEQ ID NOs: 65, 66, 67, 68, 69 and 49;
SEQ ID NOs: 70, 71, 72, 73, 74 and 54;
SEQ ID NOs: 75, 76, 77, 78, 79 and 59; or
SEQ ID NOs: 80, 81, 82, 83, 84 and 64.

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 1, 2, 3, 4, 5 and 6 (according to Kabat);
SEQ ID NOs: 25, 26, 27, 28, 29 and 6 (according to IMGT);
SEQ ID NOs: 45, 46, 47, 48, 29 and 49 (according to Chothia); or
SEQ ID NOs: 65, 66, 67, 68, 69 and 49 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 7, 8, 9, 10, 11 and 12 (according to Kabat);
SEQ ID NOs: 30, 31, 32, 33, 34 and 12 (according to IMGT);
SEQ ID NOs: 50, 51, 52, 53, 34 and 54 (according to Chothia); or
SEQ ID NOs: 70, 71, 72, 73, 74 and 54 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 13, 14, 15, 16, 17, and 18 (according to Kabat);
SEQ ID NOs: 35, 36, 37, 38, 39 and 18 (according to IMGT);
SEQ ID NOs: 55, 56, 57, 58, 39 and 59 (according to Chothia); or
SEQ ID NOs: 80, 81, 82, 83, 84 and 64 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 19, 20, 21, 22, 23 and 24 (according to Kabat);
SEQ ID NOs: 40, 41, 42, 43, 44 and 24 (according to IMGT);
SEQ ID NOs: 60, 61, 62, 62, 44 and 64 (according to Chothia); or
SEQ ID NOs: 80, 81, 82, 83, 84 and 64 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 210, 211, 212, 213, 214 and 215 (according to Kabat);
SEQ ID NOs: 222, 223, 224, 225, 226 and 215 (according to IMGT);
SEQ ID NOs: 232, 233, 234, 235, 226 and 236 (according to Chothia); or
SEQ ID NOs: 241, 242, 243, 244, 245 and 246 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 216, 217, 218, 219, 220 and 221 (according to Kabat);
SEQ ID NOs: 227, 228, 229, 230, 231 and 221 (according to IMGT);
SEQ ID NOs: 232, 237, 238, 239, 231 and 240 (according to Chothia); or
SEQ ID NOs: 247, 248, 249, 239, 250 and 251 (according to Honegger).

In various embodiments, the antibody targeting CD47 comprises a VH and a VL comprising the amino acid sequences set forth, respectively, or comprise amino acid sequences that are at least 80%, at least 85%, at least 90%, at least 91%, at least 92%, at least 93%, at least 94%, at least 95%, at least 96%, at least 97%, at least 98%, or at least 99% identical to the amino acid sequences set forth, respectively, in:
SEQ ID NOs: 85 and 86;
SEQ ID NOs: 87 and 88;
SEQ ID NOs: 89 and 90;
SEQ ID NOs: 91 and 92;
SEQ ID NOs: 252 and 253; or
SEQ ID NOs: 254 and 255. Sequence identity can be determined according to the BLAST algorithm (blast.ncbi.nlm.nih.gov/Blast.cgi), using default settings.

Amino acid sequences of CDRs and variable regions (VH/VL) of illustrative anti-CD47 antibodies that can be used in the present methods are described in Tables A1, A2, A3, A4 and B.

TABLE A1
CDRs for illustrative anti-CD47 binding antibodies (Kabat)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 1 | NYNMH | TIYPGNDDTSYNQKFKD | GGYRAMDY | RSSQSIVYSNGNTYLG | KVSNRFS | FQGSHVPYT |
| | SEQ ID NO: 1 | SEQ ID NO: 2 | SEQ ID NO: 3 | SEQ ID NO: 4 | SEQ ID | SEQ ID NO: 6 |
| | | | | | NO: 5 | |
| 2 | DYYIN | RIYPGIGNTYYNKKFKG | GHYGRGMDY | KSSQSLLNSIDQKNYLA | FASTKES | QQHYSTPWT |
| | SEQ ID NO: 7 | SEQ ID NO: 8 | SEQ ID NO: 9 | SEQ ID NO: 10 | SEQ ID | SEQ ID NO: 12 |
| | | | | | NO: 11 | |
| 3 | RAWMN | RIKRKTDGETTDYAAPV | SNRAFDI | KSSQSVLYAGNNRNYLA | QASTRAS | QQYYTPPLA |
| | SEQ ID NO: 13 | KG | SEQ ID NO: 15 | SEQ ID NO: 16 | SEQ ID | SEQ ID NO: 18 |
| | | SEQ ID NO: 14 | | | NO: 17 | |
| 4 | SYYWSW | YIYYSGSTNYNPSLKS | GKTGSAA | RASQGISRWLA | AASSLQS | QQTVSFPIT |
| | SEQ ID NO: 19 | SEQ ID NO: 20 | SEQ ID NO: 21 | SEQ ID NO: 22 | SEQ ID | SEQ ID NO: 24 |
| | | | | | NO: 23 | |
| 51 | SYWMN | MIDPSDSETHNAQKFQG | LYRWYFDV | RASEIVGTYVS | GASNRYT | GQSYNFPYT |
| | SEQ ID NO: 210 | SEQ ID NO: 211 | SEQ ID NO: 212 | SEQ ID NO: 213 | SEQ ID | SEQ ID NO: 215 |
| | | | | | NO: 214 | |
| 52 | SYYMH | IINPSGGSTSYAQKFQG | STLWFSEFDY | SGTSSDVGGHNYVS | DVTKRPS | LSYAGSRVY |
| | SEQ ID NO: 216 | SEQ ID NO: 217 | SEQ ID NO: 218 | SEQ ID NO: 219 | SEQ ID | SEQ ID NO: 221 |
| | | | | | NO: 220 | |

TABLE A2
CDRs for illustrative anti-CD47 binding antibodies (IMGT)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 5 | GYTFTNYN | IYPGNDDT | ARGGYRAMDY | QSIVYSNGNTY | KVS | FQGSHVPYT |
| | SEQ ID NO: 25 | SEQ ID NO: 26 | SEQ ID NO: 27 | SEQ ID NO: 28 | SEQ ID | SEQ ID NO: 6 |
| | | | | | NO: 29 | |
| 6 | GYSFTDYY | IYPGIGNT | ARGHYGRGMDY | QSLLNSIDQKNY | FAS | QQHYSTPWT |
| | SEQ ID NO: 30 | SEQ ID NO: 31 | SEQ ID NO: 32 | SEQ ID NO: 33 | SEQ ID | SEQ ID NO: 12 |
| | | | | | NO: 34 | |
| 7 | GLTFERAW | IKRKTDGETT | AGSNRAFDI | QSVLYAGNNRNY | QAS | QQYYTPPLA |
| | SEQ ID NO: 35 | SEQ ID NO: 36 | SEQ ID NO: 37 | SEQ ID NO: 38 | SEQ ID | SEQ ID NO: 18 |
| | | | | | NO: 39 | |
| 8 | GGSISSYY | IYYSGST | ARGKTGSAA | QGISRW | AS | QQTVSFPIT |
| | SEQ ID NO: 40 | SEQ ID NO: 41 | SEQ ID NO: 42 | SEQ ID NO: 43 | SEQ ID | SEQ ID NO: 24 |
| | | | | | NO: 44 | |
| 53 | GYTFTSYW | IDPSDSET | ARLYRWYFDV | EIVGTY | GAS | GQSYNFPYT |
| | SEQ ID NO: 222 | SEQ ID NO: 223 | SEQ ID NO: 224 | SEQ ID NO: 225 | SEQ ID | SEQ ID NO: 215 |
| | | | | | NO: 226 | |
| 54 | GYTFTSYY | INPSGGST | ARSTLWFSEFDY | SSDVGGHNY | DVT | LSYAGSRVY |
| | SEQ ID NO: 227 | SEQ ID NO: 228 | SEQ ID NO: 229 | SEQ ID NO: 230 | SEQ ID | SEQ ID NO: 221 |
| | | | | | NO: 231 | |

TABLE A3
CDRs for illustrative anti-CD47 binding antibodies (Chothia)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 9 | GYTFTNY | PGND | GYRAMD | SQSIVYSNGNTY | KVS | GSHVPY |
| | SEQ ID NO: 45 | SEQ ID NO: 46 | SEQ ID NO: 47 | SEQ ID NO: 48 | SEQ ID | SEQ ID NO: 49 |
| | | | | | NO: 29 | |
| 10 | GYSFTDY | PGIG | HYGRGMD | SQSLLNSIDQKNY | FAS | HYSTPW |
| | SEQ ID NO: 50 | SEQ ID NO: 51 | SEQ ID NO: 52 | SEQ ID NO: 53 | SEQ ID | SEQ ID NO: 54 |
| | | | | | NO: 34 | |
| 11 | GLTFERA | RKTDGE | NRAFD | SQSVLYAGNNRNY | QAS | YYTPPL |
| | SEQ ID NO: 55 | SEQ ID NO: 56 | SEQ ID NO: 57 | SEQ ID NO: 58 | SEQ ID | SEQ ID NO: 59 |
| | | | | | NO: 39 | |
| 12 | GGSISSY | YSG | KTGSA | SQGISRW | AS | TVSFPI |
| | SEQ ID NO: 60 | SEQ ID NO: 61 | SEQ ID NO: 62 | SEQ ID NO: 63 | SEQ ID | SEQ ID NO: 64 |
| | | | | | NO: 44 | |
| 55 | GYTFTSY | PSDS | YRWYFD | SEIVGTY | GAS | SYNFPY |
| | SEQ ID NO: 232 | SEQ ID NO: 233 | SEQ ID NO: 234 | SEQ ID NO: 235 | SEQ ID | SEQ ID NO: 236 |
| | | | | | NO: 226 | |
| 56 | GYTFTSY | PSGG | TLWFSEFD | GTSSDVGGHNY | DVT | YAGSRV |
| | SEQ ID NO: 232 | SEQ ID NO: 237 | SEQ ID NO: 238 | SEQ ID NO: 239 | SEQ ID | SEQ ID NO: 240 |
| | | | | | NO: 231 | |

TABLE A4
CDRs for illustrative anti-CD47 binding antibodies (Honegger)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 13 | ASGYTFTNYN | IYPGNDDTSYNQKFKDR | GGYRAMD | SSQSIVYSNGNTY | KVSNRFSGVPDR | GSHVPY |
| | SEQ ID NO: 65 | SEQ ID NO: 66 | SEQ ID | SEQ ID NO: 68 | SEQ ID NO: 69 | SEQ ID |
| | | | NO: 67 | | | NO: 49 |
| 14 | ASGYSFTDYY | IYPGIGNTYYNKKFKGR | GHYGRGMD | SSQSLLNSIDQKNY | FASTKESGVPDR | HYSTPW |
| | SEQ ID NO: 70 | SEQ ID NO: 71 | SEQ ID | SEQ ID NO: 73 | SEQ ID NO: 74 | SEQ ID |
| | | | NO: 72 | | | NO: 54 |
| 15 | ASGLTFERAW | IKRKTDGETTDYAAPVK | SNRAFD | SSQSVLYAGNNRNY | QASTRASGVPDR | YYTPPL |
| | SEQ ID NO: 75 | GR | SEQ ID | SEQ ID NO: 78 | SEQ ID NO: 79 | SEQ ID |
| | | SEQ ID NO: 76 | NO: 77 | | | NO: 59 |
| 16 | VSGGSISSYY | IYYSGSTNYNPSLKSR | GKTGSA | ASQGISRW | AASSLQSGVPSR | TVSFPI |
| | SEQ ID NO: 80 | SEQ ID NO: 81 | SEQ ID | SEQ ID NO: 83 | SEQ ID NO: 84 | SEQ ID |
| | | | NO: 82 | | | NO: 64 |
| 57 | ASGYTFTSYW | IDPSDSETHNAQKFQGK | LYRWYFD | ASEIVGTY | GASNRYTGVPAR | SYNFPY |
| | SEQ ID NO: 241 | SEQ ID NO: 242 | SEQ ID | SEQ ID NO: 244 | SEQ ID NO: 245 | SEQ ID |
| | | | NO: 243 | | | NO: 246 |
| 58 | ASGYTFTSYY | INPSGGSTSYAQKFQGR | STLWFSEFD | GTSSDVGGHNY | DVTKRPSGVPDR | YAGSRVY |
| | SEQ ID NO: 247 | SEQ ID NO: 248 | SEQ ID | SEQ ID NO: 239 | SEQ ID NO: 250 | SEQ ID |
| | | | NO: 249 | | | NO: 251 |

TABLE B
VH/VL for illustrative anti-CD47 binding antibodies

| Ab | | |
|---|---|---|
| Name | VH | VL |
| 17 | SEQ ID NO: 85 | SEQ ID NO: 86 |
| | QVQLVQSGAEVKKPGASVKVSCKASGYTFTNYNMHWVRQA | DIVMTQSPLSLPVTPGEPASISCRSSQSIVYSNGNTYL |
| | PGQRLEWMGTIYPGNDDTSYNQKFKDRVTITADTSASTAY | GWYLQKPGQSPQLLIYKVSNRFSGVPDRFSGSGSGTDF |
| | MELSSLRSEDTAVYYCARGGYRAMDYWGQGTLVTVSS | TLKISRVEAEDVGVYYCFQGSHVPYTFGQGTKLEIK |
| 18 | SEQ ID NO: 87 | SEQ ID NO: 88 |
| | QVQLVQSGAEVKKPGASVKVSCKASGYSFTDYYINWVRQA | DIVMTQSPDSLAVSLGERATINCKSSQSLLNSIDQKNY |
| | PGQGLEWMGRIYPGIGNTYYNKKFKGRVTITRDTSASTAY | LAWYQQKPGQPPKLLIYFASTKESGVPDRFSGSGSGTD |
| | MELSSLRSEDTAVYYCARGHYGRGMDYWGQGTLVTVSS | FTLTISGLQAEDVAVYFCQQHYSTPWTFGGGTKVEIR |
| 19 | SEQ ID NO: 89 | SEQ ID NO: 90 |
| | EVQLVESGGGLVKPGGSLRLSCAASGLTFERAWMNWVRQA | DIVMTQSPDSLAVSLGERATINCKSSQSVLYAGNNRNY |
| | PGKGLEWVGRIKRKTDGETTDYAAPVKGRFSISRDDSKNT | LAWYQQKPGQPPKLLINQASTRASGVPDRFSGSGSGTE |
| | LYLQMNSLKTEDTAVYYCAGSNRAFDIWGQGTMVTVSS | FTLIISSLQAEDVAIYYCQQYYTPPLAFGGGTKLEIK |
| 20 | SEQ ID NO: 91 | SEQ ID NO: 92 |
| | QVQLQESGPGLVKPSETLSLTCTVSGGSISSYYWSWIRQP | DIQMTQSPSSVSASVGDRVTITCRASQGISRWLAWYQQ |
| | PGKGLEWIGYIYYSGSTNYNPSLKSRVTISVDTSKNQFSL | KPGKAPKLLIYAASSLQSGVPSRFSGSGSGTDFTLTIS |
| | KLSSVTAADTAVYYCARGKTGSAAWGQGTLVTVSS | SLQPEDFATYYCQQTVSFPITFGGGTKVEIK |
| 59 | SEQ ID NO: 252 | SEQ ID NO: 253 |
| | QVQLVQSGAEVVKPGASVKLSCKASGYTFTSYWMNWVRQR | NIVMTQSPATMSMSPGERVTLSCRASEIVGTYVSWFQQ |
| | PGQGLEWIGMIDPSDSETHNAQKFQGKATLTVDKSTSTAY | KPGQAPRLLIYGASNRYTGVPARFSGSGSGTDFTLTIS |
| | MHLSSLRSEDTAVYYCARLYRWYFDVWGAGTTVTVSS | SVQPEDLADYHCGQSYNFPYTFGGGTKLEIK |
| 60 | SEQ ID NO: 254 | SEQ ID NO: 255 |
| | QVQLVQSGAEVKKPGASVKVSCKASGYTFTSYYMHWVRQA | QSVLTQPSSVSASPGQSITISCSGTSSDVGGHNYVSWY |
| | PGQGLEWMGIINPSGGSTSYAQKFQGRVTMTRDTSTSTVY | QQHPGKAPKLMIYDVTKRPSGVPDRFSGSKSGNTASLT |
| | MELSSLRSEDTAVYYCARSTLWFSEFDYWGQGTLVTVSS | VSGLQAEDEADYYCLSYAGSRVYVFGTGTKLTVL |

Additional anti-CD47 antibodies of use in the present methods include those described in WO199727873, WO199940940, WO2002092784, WO2005044857, WO2009046541, WO2010070047, WO2011143624, WO2012170250, WO2013109752, WO2013119714, WO2014087248, WO2015191861, WO2016022971, WO2016023040, WO2016024021, WO2016081423, WO2016109415, WO2016141328, WO2016188449, WO2017027422, WO2017049251, WO2017053423, WO2017121771, WO2017194634, WO2017196793, WO2017215585, WO2018075857, WO2018075960, WO2018089508, WO2018095428, WO2018137705, WO2018233575, WO2019027903, WO2019034895, WO2019042119, WO2019042285, WO2019042470, WO2019086573, WO2019108733, WO2019138367, WO2019144895, WO2019157843, WO2019179366, WO2019184912, WO2019185717, WO2019201236, WO2019238012, WO2019241732, WO2020019135, WO2020036977, WO2020043188 and WO2020009725.

ii. Antibody or Antigen-Binding Fragment Thereof that Binds to SIRPα

In various embodiments, the agent that inhibits binding between CD47 and SIRPα CD47 is an antibody or antigen-binding fragment thereof that binds to signal regulatory protein alpha (SIRPα) (NCBI Gene ID: 140885; UniProt P78324). Illustrative antibodies that bind to SIRPα include without limitation GS-0189 (FSI-189), ES-004, B1765063, ADU1805, and CC-95251.

In certain aspects, an antibody can comprise one or more CDRs of 1H9. In certain aspects, an antibody can comprise all CDRs of 1H9. In certain aspects, an antibody can comprise one or more variable sequences of 1H9. In certain aspects, an antibody can comprise each variable sequence of 1H9. In certain aspects, an antibody can comprise the heavy chain of 1H9. In certain aspects, an antibody can comprise the light chain of 1H9. In certain aspects, an antibody can comprise the heavy chain and the light chain of 1H9. In certain aspects, an antibody is 1H9.

In certain aspects, an antibody can comprise one or more CDRs of 3C2. In certain aspects, an antibody can comprise all CDRs of 3C2. In certain aspects, an antibody can comprise one or more variable sequences of 3C2. In certain aspects, an antibody can comprise each variable sequence of 3C2. In certain aspects, an antibody can comprise the heavy chain of 3C2. In certain aspects, an antibody can comprise the light chain of 3C2. In certain aspects, an antibody can comprise the heavy chain and the light chain of 3C2. In certain aspects, an antibody is 3C2.

In certain aspects, an antibody can comprise one or more CDRs of 9B11. In certain aspects, an antibody can comprise all CDRs of 9B11. In certain aspects, an antibody can comprise one or more variable sequences of 9B11. In certain aspects, an antibody can comprise each variable sequence of 9B11. In certain aspects, an antibody can comprise the heavy chain of 9B11. In certain aspects, an antibody can comprise the light chain of 9B11. In certain aspects, an antibody can comprise the heavy chain and the light chain of 9B11. In certain aspects, an antibody is 9B11.

In certain aspects, an antibody can comprise one or more CDRs of 7E11. In certain aspects, an antibody can comprise all CDRs of 7E11. In certain aspects, an antibody can comprise one or more variable sequences of 7E11. In certain aspects, an antibody can comprise each variable sequence of 7E11. In certain aspects, an antibody can comprise the heavy chain of 7E11. In certain aspects, an antibody can comprise the light chain of 7E11. In certain aspects, an antibody can comprise the heavy chain and the light chain of 7E11. In certain aspects, an antibody is 7E11.

Additional anti-SIRPα antibodies of use in the present methods include those described in WO200140307, WO2002092784, WO2007133811, WO2009046541, WO2010083253, WO2011076781, WO2013056352, WO2015138600, WO2016179399, WO2016205042, WO2017178653, WO2018026600, WO2018057669, WO2018107058, WO2018190719, WO2018210793, WO2019023347, WO2019042470, WO2019175218, WO2019183266, WO2020013170, WO2020068752 and WO2020088580.

In various embodiments, the antibody targeting CD47 comprises a VH-CDR1, a VH-CDR2, a VH-CDR3, a VL-CDR1, a VL-CDR2 and a VL-CDR3 comprising the following amino acid sequences (according to Kabat), respectively:
SEQ ID NOs: 93, 94, 95, 96, 97 and 98;
SEQ ID NOs: 99, 100, 101, 102, 103 and 104;
SEQ ID NOs: 99, 100, 105, 102, 103 and 106;
SEQ ID NOs: 107, 108, 109, 110, 111 and 112;
SEQ ID NOs: 113, 114, 115, 116, 117 and 118; or
SEQ ID NOs: 119, 120, 121, 122, 123 and 124.

In various embodiments, the antibody targeting CD47 comprises a VH-CDR1, a VH-CDR2, a VH-CDR3, a VL-CDR1, a VL-CDR2 and a VL-CDR3 comprising the following amino acid sequences (according to IMGT), respectively:
SEQ ID NOs: 125, 126, 127, 128, 129 and 98;
SEQ ID NOs: 125, 130, 131, 132, 29 and 104;
SEQ ID NOs: 125, 130, 133, 132, 29 and 106;
SEQ ID NOs: 134, 135, 136, 137, 138 and 112;
SEQ ID NOs: 139, 130, 140, 141, 142 and 118; or
SEQ ID NOs: 143, 144, 145, 146, 44 and 124.

In various embodiments, the antibody targeting CD47 comprises a VH-CDR1, a VH-CDR2, a VH-CDR3, a VL-CDR1, a VL-CDR2 and a VL-CDR3 comprising the following amino acid sequences (according to Chothia), respectively:
SEQ ID NOs: 147, 148, 149, 150, 129 and 151;
SEQ ID NOs: 147, 152, 153, 154, 29 and 155;
SEQ ID NOs: 147, 152, 156, 154, 29 and 157;
SEQ ID NOs: 158, 159, 160, 161, 138 and 162;
SEQ ID NOs: 163, 152, 164, 165, 142 and 166; or
SEQ ID NOs: 167, 168, 169, 170, 44 and 171.

In various embodiments, the antibody targeting CD47 comprises a VH-CDR1, a VH-CDR2, a VH-CDR3, a VL-CDR1, a VL-CDR2 and a VL-CDR3 comprising the following amino acid sequences (according to Honegger), respectively:
SEQ ID NOs: 172, 173, 174, 175, 176 and 151;
SEQ ID NOs: 172, 177, 178, 179, 180 and 155;
SEQ ID NOs: 172, 181, 182, 179, 180 and 157;
SEQ ID NOs: 183, 184, 185, 186, 187 and 162;
SEQ ID NOs: 188, 189, 190, 191, 192 and 166; or
SEQ ID NOs: 193, 194, 195, 196, 197 and 171.

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 93, 94, 95, 96, 97 and 98 (according to Kabat);
SEQ ID NOs: 125, 126, 127, 128, 129 and 98 (according to IMGT);
SEQ ID NOs: 147, 148, 149, 150, 129 and 151 (according to Chothia); or
SEQ ID NOs: 172, 173, 174, 175, 176 and 151 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 99, 100, 101, 102, 103 and 104 (according to Kabat);
SEQ ID NOs: 125, 130, 131, 132, 29 and 104 (according to IMGT);
SEQ ID NOs: 147, 152, 153, 154, 29 and 155 (according to Chothia); or
SEQ ID NOs: 172, 177, 178, 179, 180 and 155 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 99, 100, 105, 102, 103 and 106 (according to Kabat);
SEQ ID NOs: 125, 130, 133, 132, 29 and 106 (according to IMGT);
SEQ ID NOs: 147, 152, 156, 154, 29 and 157 (according to Chothia); or
SEQ ID NOs: 172, 181, 182, 179, 180 and 157 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 107, 108, 109, 110, 111 and 112 (according to Kabat);
SEQ ID NOs: 134, 135, 136, 137, 138 and 112 (according to IMGT);
SEQ ID NOs: 158, 159, 160, 161, 138 and 162 (according to Chothia); or
SEQ ID NOs: 183, 184, 185, 186, 187 and 162 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 113, 114, 115, 116, 117 and 118 (according to Kabat);
SEQ ID NOs: 139, 130, 140, 141, 142 and 118 (according to IMGT);
SEQ ID NOs: 163, 152, 164, 165, 142 and 166 (according to Chothia); or
SEQ ID NOs: 188, 189, 190, 191, 192 and 166 (according to Honegger).

In various embodiments, the antibody targeting CD47 comprises a VH-CDR1, a VH-CDR2, a VH-CDR3, a VL-CDR1, a VL-CDR2 and a VL-CDR3 comprising the following amino acid sequences, respectively:
SEQ ID NOs: 119, 120, 121, 122, 123 and 124 (according to Kabat);
SEQ ID NOs: 143, 144, 145, 146, 44 and 124 (according to IMGT);
SEQ ID NOs: 167, 168, 169, 170, 44 and 171 (according to Chothia); or
SEQ ID NOs: 193, 194, 195, 196, 197 and 171 (according to Honegger).

In various embodiments, the antibody targeting CD47 comprises a VH and a VL comprising the amino acid sequences set forth, respectively, or comprise amino acid sequences that are at least 80%, at least 85%, at least 90%, at least 91%, at least 92%, at least 93%, at least 94%, at least 95%, at least 96%, at least 97%, at least 98%, or at least 99% identical to the amino acid sequences set forth, respectively, in:
SEQ ID NOs: 198 and 199;
SEQ ID NOs: 200 and 201;
SEQ ID NOs: 202 and 203;
SEQ ID NOs: 204 and 205;
SEQ ID NOs: 206 and 207; or
SEQ ID NOs: 208 and 209.

Amino acid sequences of CDRs and variable regions (VH/VL) of illustrative anti-SIRPα antibodies that can be used in the present methods are described in Tables C1, C2, C3, C4 and D.

TABLE C1
CDRs for illustrative anti-SIRPα binding antibodies (Kabat)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 21 | SYWIT | DIYPGSGSTNHIEKFKS | GYGSSYGYFDY | RASENIYSYLA | TAKTLAE | QHQYGPPFT |
| | SEQ ID | SEQ ID NO: 94 | SEQ ID NO: 95 | SEQ ID NO: 96 | SEQ ID | SEQ ID NO: 98 |
| | NO: 93 | | | | NO: 97 | |
| 22 | SYWMH | NIDPSDSDTHYNQKFKD | GYSKYYAMDY | RSSQSIVHSYGNTYLE | KVSNRFS | FQGSHVPYT |
| | SEQ ID | SEQ ID NO: 100 | SEQ ID | SEQ ID NO: 102 | SEQ ID | SEQ ID NO: 104 |
| | NO: 99 | | NO: 101 | | NO: 103 | |
| 23 | SYWMH | NIDPSDSDTHYNQKFKD | YGNYGENAMDY | RSSQSIVHSYGNTYLE | KVSNRFS | FQGSHVPFT |
| | SEQ ID | SEQ ID NO: 100 | SEQ ID | SEQ ID NO: 102 | SEQ ID | SEQ ID NO: 106 |
| | NO: 99 | | NO: 105 | | NO: 103 | |
| 24 | DYYIH | RIDPEDGETKYAPKFQG | GGFAY | ASSSVSSSYLY | STSNLAS | HQWSSHPYT |
| | SEQ ID | SEQ ID NO: 108 | SEQ ID | SEQ ID NO: 110 | SEQ ID | SEQ ID NO: 112 |
| | NO: 107 | | NO: 109 | | NO: 111 | |
| 25 | SYWVH | NIDPSDSDTHYSPSFQG | GGTGTLAYFAY | RSSQSLVHSYGNTYLY | RVSNRFS | FQGTHVPYT |
| | SEQ ID | SEQ ID NO: 114 | SEQ ID | SEQ ID NO: 116 | SEQ ID | SEQ ID NO: 118 |
| | NO: 113 | | NO: 115 | | NO: 117 | |
| 26 | GYGIS | WISAYGGETNYAQKLQG | EAGSSWYDFDL | RASQGISSWLA | AASNLQS | QQGASFPIT |
| | SEQ ID | SEQ ID NO: 120 | SEQ ID | SEQ ID NO: 122 | SEQ ID | SEQ ID NO: 124 |
| | NO: 119 | | NO: 121 | | NO: 123 | |

TABLE C2
CDRs for illustrative anti-SIRPα binding antibodies (IMGT)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 27 | GYTFTSYW | IYPGSGST | ATGYGSSYGYFDY | ENIYSY | TAK | QHQYGPPFT |
| | SEQ ID NO: 125 | SEQ ID NO: 126 | SEQ ID NO: 127 | SEQ ID NO: 128 | SEQ ID | SEQ ID NO: 98 |
| | | | | | NO: 129 | |
| 28 | GYTFTSYW | IDPSDSDT | ARGYSKYYAMDY | QSIVHSYGNTY | KVS | FQGSHVPYT |
| | SEQ ID NO: 125 | SEQ ID NO: 130 | SEQ ID NO: 131 | SEQ ID NO: 132 | SEQ ID | SEQ ID NO: 104 |
| | | | | | NO: 29 | |
| 29 | GYTFTSYW | IDPSDSDT | ASYGNYGENAMDY | QSIVHSYGNTY | KVS | FQGSHVPFT |
| | SEQ ID NO: 125 | SEQ ID NO: 130 | SEQ ID NO: 133 | SEQ ID NO: 132 | SEQ ID | SEQ ID NO: 106 |
| | | | | | NO: 29 | |
| 30 | GFNIKDYY | IDPEDGET | AKGGFAY | SSVSSSY | STS | HQWSSHPYT |
| | SEQ ID NO: 134 | SEQ ID NO: 135 | SEQ ID NO: 136 | SEQ ID NO: 137 | SEQ ID | SEQ ID NO: 112 |
| | | | | | NO: 138 | |
| 31 | GYSFTSYW | IDPSDSDT | VRGGTGTLAYFAY | QSLVHSYGNTY | RVS | FQGTHVPYT |
| | SEQ ID NO: 139 | SEQ ID NO: 130 | SEQ ID NO: 140 | SEQ ID NO: 141 | SEQ ID | SEQ ID NO: 118 |
| | | | | | NO: 142 | |
| 32 | GYTFRGYG | ISAYGGET | AREAGSSWYDFDL | QGISSW | AS | QQGASFPIT |
| | SEQ ID NO: 143 | SEQ ID NO: 144 | SEQ ID NO: 145 | SEQ ID NO: 146 | SEQ ID | SEQ ID NO: 124 |
| | | | | | NO: 44 | |

TABLE C3
CDRs for illustrative anti-SIRPα binding antibodies (Chothia)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 33 | GYTFTSY | PGSG | YGSSYGYFD | SENIYSY | TAK | QYGPPF |
| | SEQ ID NO: 147 | SEQ ID NO: 148 | SEQ ID NO: 149 | SEQ ID NO: 150 | SEQ ID NO: 129 | SEQ ID NO: 151 |
| 34 | GYTFTSY | PSDS | YSKYYAMD | SQSIVHSYGNTY | KVS | GSHVPY |
| | SEQ ID NO: 147 | SEQ ID NO: 152 | SEQ ID NO: 153 | SEQ ID NO: 154 | SEQ ID NO: 29 | SEQ ID NO: 155 |
| 35 | GYTFTSY | PSDS | GNYGENAMD | SQSIVHSYGNTY | KVS | GSHVPF |
| | SEQ ID NO: 147 | SEQ ID NO: 152 | SEQ ID NO: 156 | SEQ ID NO: 154 | SEQ ID NO: 29 | SEQ ID NO: 157 |
| 36 | GFNIKDY | PEDG | GFA | SSSVSSSY | STS | WSSHPY |
| | SEQ ID NO: 158 | SEQ ID NO: 159 | SEQ ID NO: 160 | SEQ ID NO: 161 | SEQ ID NO: 138 | SEQ ID NO: 162 |
| 37 | GYSFTSY | PSDS | GTGTLAYFA | SQSLVHSYGNTY | RVS | GTHVPY |
| | SEQ ID NO: 163 | SEQ ID NO: 152 | SEQ ID NO: 164 | SEQ ID NO: 165 | SEQ ID NO: 142 | SEQ ID NO: 166 |
| 38 | GYTFRGY | AYGG | AGSSWYDFD | SQGISSW | AS | GASFPI |
| | SEQ ID NO: 167 | SEQ ID NO: 168 | SEQ ID NO: 169 | SEQ ID NO: 170 | SEQ ID NO: 44 | SEQ ID NO: 171 |

TABLE C4
CDRs for illustrative anti-SIRPα binding antibodies (Honegger)

| Ab | | | | | | |
|---|---|---|---|---|---|---|
| Name | VH-CDR1 | VH-CDR2 | VH-CDR3 | VL-CDR1 | VL-CDR2 | VL-CDR3 |
| 39 | ASGYTFTSYW | IYPGSGSTNHIEKFKSK | GYGSSYGYFD | ASENIYSY | TAKTLAEGVPSR | QYGPPF |
| | SEQ ID NO: 172 | SEQ ID NO: 173 | SEQ ID NO: 174 | SEQ ID NO: 175 | SEQ ID NO: 176 | SEQ ID |
| | | | | | | NO: 151 |
| 40 | ASGYTFTSYW | IDPSDSDTHYNQKFKDR | GYSKYYAMD | SSQSIVHSYGNTY | KVSNRFSGVPDR | GSHVPY |
| | SEQ ID NO: 172 | SEQ ID NO: 177 | SEQ ID NO: 178 | SEQ ID NO: 179 | SEQ ID NO: 180 | SEQ ID |
| | | | | | | NO: 155 |
| 41 | ASGYTFTSYW | IDPSDSDTHYNQKFKDK | YGNYGENAMD | SSQSIVHSYGNTY | KVSNRFSGVPDR | GSHVPF |
| | SEQ ID NO: 172 | SEQ ID NO: 181 | SEQ ID NO: 182 | SEQ ID NO: 179 | SEQ ID NO: 180 | SEQ ID |
| | | | | | | NO: 157 |
| 42 | ASGFNIKDYY | IDPEDGETKYAPKFQGK | GGFA | ASSSVSSSY | STSNLASGVPAR | WSSHPY |
| | SEQ ID NO: 183 | SEQ ID NO: 184 | SEQ ID NO: 185 | SEQ ID NO: 186 | SEQ ID NO: 187 | SEQ ID |
| | | | | | | NO: 162 |
| 43 | ASGYSFTSYW | IDPSDSDTHYSPSFQGH | GGTGTLAYFA | SSQSLVHSYGNTY | RVSNRFSGVPDR | GTHVPY |
| | SEQ ID NO: 188 | SEQ ID NO: 189 | SEQ ID NO: 190 | SEQ ID NO: 191 | SEQ ID NO: 192 | SEQ ID |
| | | | | | | NO: 166 |
| 44 | ASGYTFRGYG | ISAYGGETNYAQKLQGR | EAGSSWYDFD | ASQGISSW | AASNLQSGVPSR | GASFPI |
| | SEQ ID NO: 193 | SEQ ID NO: 194 | SEQ ID NO: 195 | SEQ ID NO: 196 | SEQ ID NO: 197 | SEQ ID |
| | | | | | | NO: 171 |

TABLE D
VH/VL for illustrative anti-SIRPα binding antibodies

| Ab Name | VH | VL |
|---|---|---|
| 45 | SEQ ID NO: 198 | SEQ ID NO: 199 |
| | QVQLVQSGAEVKKPGASVKVSCKASGYTFTSYWITWVKQAP | DIQMTQSPSSLSASVGDRVTITCRASENIYSYLAWYQQ |
| | GQGLEWIGDIYPGSGSTNHIEKFKSKATLTVDTSISTAYME | KPGKAPKLLIYTAKTLAEGVPSRFSGSGSGTDFTLTIS |
| | LSRLRSDDTAVYYCATGYGSSYGYFDYWGQGTLVTVSS | SLQPEDFATYYCQHQYGPPFTFGQGTKLEIK |
| 46 | SEQ ID NO: 200 | SEQ ID NO: 201 |
| | QVQLVQSGAEVKKPGASVKVSCKASGYTFTSYWMHWVRQAP | DIVMTQTPLSLSVTPGQPASISCRSSQSIVHSYGNTYL |
| | GQGLEWMGNIDPSDSDTHYNQKFKDRVTMTRDTSTSTVYME | EWYLQKPGQSPQLLIYKVSNRFSGVPDRFSGSGSGTDF |
| | LSSLRSEDTAVYYCARGYSKYYAMDYWGQGTLVTVSS | TLKISRVEAEDVGVYYCFQGSHVPYTFGQGTKLEIK |
| 47 | SEQ ID NO: 202 | SEQ ID NO: 203 |
| | QVKLQESGAELVRPGSSVKLSCKASGYTFTSYWMHWVKQRP | DILMTQTPLSLPVSLGDQASISCRSSQSIVHSYGNTYL |
| | IQGLEWIGNIDPSDSDTHYNQKFKDKATLTVDNSSSTAYMQ | EWYLQKPGQSPKLLIYKVSNRFSGVPDRFSGSGSGTDF |
| | LSSLTSEDSAVYYCASYGNYGENAMDYWGQGTSVTVSS | TLKISRVEAEDLGVYYCFQGSHVPFTFGSGTKLEIK |
| 48 | SEQ ID NO: 204 | SEQ ID NO: 205 |
| | EVQLQQSGAELVKPGASVKLSCTASGFNIKDYYIHWVKQRT | QIVLTQSPAIMSASPGEKVTLTCSASSSVSSSYLYWYQ |
| | EQGLEWIGRIDPEDGETKYAPKFQGKATITADTSSNTAYLQ | QKPGSSPKLWIYSTSNLASGVPARFSGSGSGTSYSLTI |
| | LNSLTSEDTAVYSCAKGGFAYWGQGTLVTVSA | SSMEAEDAASYFCHQWSSHPYTFGGGTKLEIK |
| 49 | SEQ ID NO: 206 | SEQ ID NO: 207 |
| | EVQLVQSGAEVKKPGESLRISCKASGYSFTSYWVHWVRQMP | DVVMTQSPLSLPVTLGQPASISCRSSQSLVHSYGNTYL |
| | GKGLEWMGNIDPSDSDTHYSPSFQGHVTLSVDKSISTAYLQ | YWFQQRPGQSPRLLIYRVSNRFSGVPDRFSGSGSGTDF |
| | LSSLKASDTAMYYCVRGGTGTLAYFAYWGQGTLVTVSS | TLKISRVEAEDVGVYYCFQGTHVPYTFGGGTKVEIK |
| 50 | SEQ ID NO: 208 | SEQ ID NO: 209 |
| | QVQLVQSGAEVKKPGASVKVSCKASGYTFRGYGISWVRQAP | DIQMTQSPSSVSASVGDRVTITCRASQGISSWLAWYQQ |
| | GQGLEWMGWISAYGGETNYAQKLQGRVTMTTDTSTSTAYME | KPGKAPKLLIYAASNLQSGVPSRFSGSGSGTDFTLTIS |
| | LRSLRSDDTAVYYCAREAGSSWYDFDLWGRGTLVTVSS | SLQPEDFATYYCQQGASFPITFGGGTKVEIK |

iii. SIRPα-Fc Fusion Protein

In various embodiments, the agent that inhibits binding between CD47 and SIRPα CD47 is a SIRPα-Fc fusion protein or a “high affinity SIRPα reagent”, which includes SIRPα-derived polypeptides and analogs thereof. High affinity SIRPα reagents are described in international application WO2013109752A1, which is hereby specifically incorporated by reference. High affinity SIRPα reagents are variants of the native SIRPα protein. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide binding to CD47, for example by decreasing the off-rate by at least 10-fold, at least 20-fold, at least 50-fold, at least 100-fold, at least 500-fold, or more.

A high affinity SIRPα reagent comprises the portion of SIRPα that is sufficient to bind CD47 at a recognizable affinity, e.g., high affinity, which normally lies between the signal sequence and the transmembrane domain, or a fragment thereof that retains the binding activity. The high affinity SIRPα reagent will usually comprise at least the dl domain of SIRPα with modified amino acid residues to increase affinity. In some embodiments, a SIRPα variant is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. The Fc region aids in phagocytosis by providing an “eat me” signal, which enhances the block of the “don't eat me” signal provided by the high affinity SIRPα reagent. In other embodiments, the second polypeptide is any suitable polypeptide that is substantially similar to Fc, e.g., providing increased size, multimerization domains, and/or additional binding or interaction with 1g molecules. The amino acid changes that provide for increased affinity are localized in the dl domain, and thus high affinity SIRPα reagents comprise a dl domain of human SIRPα, with at least one amino acid change relative to the wild-type sequence within the dl domain. Such a high affinity SIRPα reagent optionally comprises additional amino acid sequences, for example antibody Fc sequences; portions of the wild-type human SIRPα protein other than the dl domain, including without limitation residues 150 to 374 of the native protein or fragments thereof, usually fragments contiguous with the dl domain; and the like. High affinity SIRPα reagents may be monomeric or multimeric, i.e., dimer, trimer, tetramer, etc.

Illustrative SIRPα-Fc fusion proteins of use include ALX-148 (a.k.a., evorpacept, described in WO2013109752), TTI-621 or TTI-622 (described in WO2014094122), SIRPa-F8, JY002-M2G1 (N297A), JMT601 (CP0107), SS002M91, SIRPalpha-IgG4-Fc-Fc, and hCD172a (SIRPα)-Fc-LIGHT.

b. NEDD8-Activating Enzyme E1 Regulatory Subunit (NAE1) Inhibitor

The methods described herein entail the administration of a NEDD8-activating enzyme E1 regulatory subunit (NAE1) inhibitor. NAE1 has been assigned NCBI Gene ID: 8883 and Uniprot Accession No. Q13564. Illustrative NAE1 inhibitors include without limitation pevonedistat, TAK-243 and TAS-4464.

In some embodiments, the NAE1 inhibitor is pevonedistat. The CAS number of pevonedistat is 905579-51-3. The IUPAC Name of pevonedistat is [(1S,2S,4R)-4-[4-[[(1S)-2,3-dihydro-1H-inden-1-yl]amino]pyrrolo[2,3-d]pyrimidin-7-yl]-2-hydroxycyclopentyl]methyl sulfamate. The structure of pevonedistat is provided below.

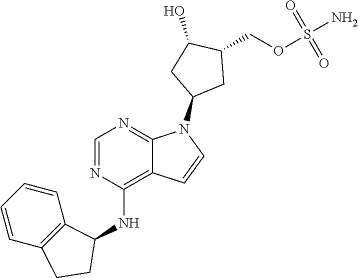 [SMILES: NS(=O)(=O)OC[C@@H]1C[C@@H](n2ccc3c(N[C@H]4CCc5ccccc54)ncnc32)C[C@@H]1O]

In some embodiments, the NAE1 inhibitor is TAK-243. The CAS number of TAK-243 is 1450833-55-2. The IUPAC Name of TAK-243 is [(1R,2R,3S,4R)-2,3-dihydroxy-4-[[2-[3-(trifluoromethylsulfanyl)phenyl]pyrazolo[1,5-a]pyrimidin-7-yl]amino]cyclopentyl]methyl sulfamate. The structure of TAK-243 is provided below.

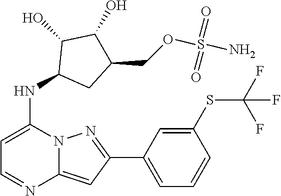 [SMILES: NS(=O)(=O)OC[C@H]1C[C@@H](Nc2ccnc3cc(-c4cccc(SC(F)(F)F)c4)nn23)[C@H](O)[C@@H]1O]

In some embodiments, the NAE1 inhibitor is TAS-4464. The CAS number of TAS-4464 is 1848959-10-3. The IUPAC name of TAS-4464 is 7H-Pyrrolo[2,3-d]pyrimidin-4-amine, 7-[5-[(aminosulfonyl)amino]-5-deoxy-beta-D-ribofuranosyl]-5-[2-(2-ethoxy-6-fluorophenyl)ethynyl]-. The structure of TAS-4464 is provided below.

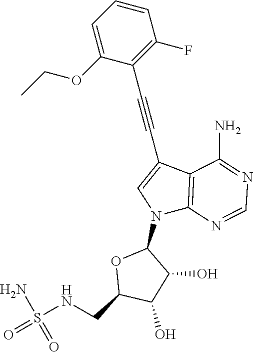 [SMILES: CCOc1cccc(F)c1C#Cc1cn([C@@H]2O[C@H](CNS(N)(=O)=O)[C@@H](O)[C@H]2O)c2ncnc(N)c12]

c. Hypomethylating Agents

In various embodiments, the methods described herein can include administration of a hypomethylating agent. Hypomethylating agents include, but are not limited to, azacitidine (Vidaza, also known as azacytidine), decitabine (Dacogen), oral decitabine and cedazuridine (ASTX727) and guadecitabine (SGI-110). In some embodiments, the hypomethylating agent is azacitidine, decitabine, decitabine/cedazuridine or guadecitabine. In some embodiments, the hypomethylating agent is azacitidine. In various embodiments, the hypomethylating agent can be administered orally, intravenously or subcutaneously, as appropriate.

Azacitidine (5-azacytidine) is a chemical analogue of cytidine and is approved by the U.S. FDA for use in the treatment of myelodysplastic syndrome (MDS). Azacitidine removes methyl groups on DNA and also inhibits DNA methyltransferase, causing hypomethylation of DNA. At higher concentrations, azacitidine incorporates into DNA and RNA, resulting in direct cytotoxicity of abnormal hematopoietic cells in the bone marrow. The structure of azacitidine is shown below:

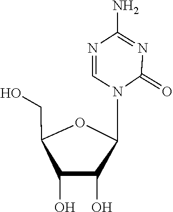

Decitabine (5-aza-2′deoxycitidine) is a chemical analogue of cytidine and is approved by the U.S. FDA for use in the treatment of myelodysplastic syndrome (MDS) and acute myeloid leukemia (AML). Similar to azacitidine, decitabine inhibits DNA methyltransferase, causing hypomethylation of DNA. However, decitabine is only integrated into DNA strands. Once integrated into DNA, decitabine binds irreversibly to DNA methyltransferases (DNMTs) and inhibits disengagement of the DNMTs from the DNA strand, resulting in inhibition of methylation of the DNA. The structure of decitabine is shown below:

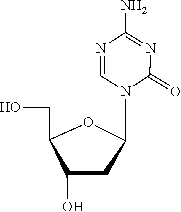

Guadecitabine sodium (SGI-110 sodium) is a second-generation DNA methyltransferases (DNMT) inhibitor. The CAS number of guadecitabine is 929901-49-5. The IUPAC name of guadecitabine is [(2R,3S, 5R)-5-(2-amino-6-oxo-1H-purin-9-yl)-3-hydroxyoxolan-2-yl]methyl [(2R,3S,5R)-5-(4-amino-2-oxo-1,3,5-triazin-1-yl)-2-(hydroxymethyl)oxolan-3-yl] hydrogen phosphate. The structure of guadecitabine sodium is shown below. Other alkali metal salts (e.g., lithium, sodium, potassium) of guadecitabine may also be of use.

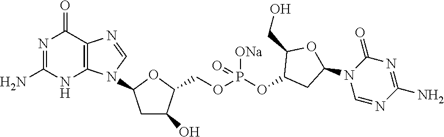

d. Additional Combination Agents

Additional agents, such as small molecules, antibodies, adoptive cellular therapies and chimeric antigen receptor T cells (CAR-T), checkpoint inhibitors, and vaccines, that are appropriate for treating hematological malignancies can be administered in combination with the agent that inhibits binding between CD47 and SIRPα (e.g., magrolimab); and the NEDD8-activating enzyme E1 regulatory subunit (NAE1) inhibitor (e.g., pevonedistat), as described herein. Additional immunotherapeutic agents for hematological malignancies are described in Dong, et al, J Life Sci (Westlake Village). 2019 June; 1(1): 46-52; and Cuesta-Mateos, et al, Front. Immunol. 8:1936. doi: 10.3389/fimmu.2017.01936, each of which are hereby incorporated by reference in their entireties for all purposes.

In various embodiments, the agent that inhibits binding between CD47 and SIRPα (e.g., magrolimab); and the NAE1 inhibitor (e.g., pevonedistat), as described herein, is further combined with one or more additional therapeutic agents, e.g., an inhibitory immune checkpoint blocker or inhibitor, a stimulatory immune checkpoint stimulator, agonist or activator, a chemotherapeutic agent, an anti-cancer agent, a radiotherapeutic agent, an anti-neoplastic agent, an anti-proliferation agent, an anti-angiogenic agent, an anti-inflammatory agent, an immunotherapeutic agent, a therapeutic antigen-binding molecule (mono- and multi-specific antibodies and fragments thereof in any format (e.g., including without limitation DARTs®, Duobodies®, BiTEs®, BiKEs, TriKEs, XmAbs®, TandAbs®, scFvs, Fabs, Fab derivatives), bi-specific antibodies, non-immunoglobulin antibody mimetics (e.g., including without limitation adnectins, affibody molecules, affilins, affimers, affitins, alphabodies, anticalins, peptide aptamers, armadillo repeat proteins (ARMs), atrimers, avimers, designed ankyrin repeat proteins (DARPins®), fynomers, knottins, Kunitz domain peptides, monobodies, and nanoCLAMPs), antibody-drug conjugates (ADC), antibody-peptide conjugate), an oncolytic virus, a gene modifier or editor, a cell comprising a chimeric antigen receptor (CAR), e.g., including a T cell immunotherapeutic agent, an NK-cell immunotherapeutic agent, or a macrophage immunotherapeutic agent, a cell comprising an engineered T-cell receptor (TCR-T), or any combination thereof.

In various embodiments, the agent that inhibits binding between CD47 and SIRPα (e.g., magrolimab); and the NAE1 inhibitor (e.g., pevonedistat), as described herein, is further combined with one or more additional therapeutic agents including, without limitation, an inhibitor, agonist, antagonist, ligand, modulator, stimulator, blocker, activator or suppressor of a target (e.g., polypeptide or polynucleotide) including without limitation: Abelson murine leukemia viral oncogene homolog 1 gene (ABL, such as ABL1), Acetyl-CoA carboxylase (such as ACC1/2), activated CDC kinase (ACK, such as ACK1), Adenosine deaminase, adenosine receptor (such as A2BR, A2aR, A3aR), Adenylate cyclase, ADP ribosyl cyclase-1, adrenocorticotropic hormone receptor (ACTH), Aerolysin, AKT1 gene, Alk-5 protein kinase, Alkaline phosphatase, Alpha 1 adrenoceptor, Alpha 2 adrenoceptor, Alpha-ketoglutarate dehydrogenase (KGDH), Aminopeptidase N, AMP activated protein kinase, anaplastic lymphoma kinase (ALK, such as ALK1), Androgen receptor, Angiopoietin (such as ligand-1, ligand-2), Angiotensinogen (AGT) gene, murine thymoma viral oncogene homolog 1 (AKT) protein kinase (such as AKT1, AKT2, AKT3), apolipoprotein A-I (APOA1) gene, Apoptosis inducing factor, apoptosis protein (such as 1, 2), apoptosis signal-regulating kinase (ASK, such as ASK1), Arginase (I), Arginine deiminase, Aromatase, Asteroid homolog 1 (ASTE1) gene, ataxia telangiectasia and Rad 3 related (ATR) serine/threonine protein kinase, Aurora protein kinase (such as 1, 2), Axl tyrosine kinase receptor, 4-1BB ligand (CD137L), Baculoviral IAP repeat containing 5 (BIRC5) gene, Basigin, B-cell lymphoma 2 (BCL2) gene, Bcl2 binding component 3, Bcl2 protein, BCL2L11 gene, BCR (breakpoint cluster region) protein and gene, Beta adrenoceptor, Beta-catenin, B-lymphocyte antigen CD19, B-lymphocyte antigen CD20, B-lymphocyte cell adhesion molecule, B-lymphocyte stimulator ligand, Bone morphogenetic protein-10 ligand, Bone morphogenetic protein-9 ligand modulator, Brachyury protein, Bradykinin receptor, B-Raf proto-oncogene (BRAF), Brc-Abl tyrosine kinase, Bromodomain and external domain (BET) bromodomain containing protein (such as BRD2, BRD3, BRD4), Bruton's tyrosine kinase (BTK), Calmodulin, calmodulin-dependent protein kinase (CaMK, such as CAMKII), Cancer testis antigen 2, Cancer testis antigen NY-ESO-1, cancer/testis antigen 1B (CTAG1) gene, Cannabinoid receptor (such as CB1, CB2), Carbonic anhydrase, casein kinase (CK, such as CKI, CKII), Caspase (such as caspase-3, caspase-7, Caspase-9), caspase 8 apoptosis-related cysteine peptidase CASP8-FADD-like regulator, Caspase recruitment domain protein-15, Cathepsin G, CCR5 gene, CDK-activating kinase (CAK), Checkpoint kinase (such as CHK1, CHK2), chemokine (C-C motif) receptor (such as CCR2, CCR4, CCR5, CCR8), chemokine (C-X-C motif) receptor (such as CXCR1, CXCR2, CXCR3 and CXCR4), Chemokine CC21 ligand, Cholecystokinin CCK2 receptor, Chorionic gonadotropin, c-Kit (tyrosine-protein kinase Kit or CD117), CISH (Cytokine-inducible SH2-containing protein), Claudin (such as 6, 18), cluster of differentiation (CD) such as CD4, CD27, CD29, CD30, CD33, CD37, CD40, CD40 ligand receptor, CD40 ligand, CD40LG gene, CD44, CD45, CD47, CD49b, CD51, CD52, CD55, CD58, CD66e (CEACAM6), CD70 gene, CD74, CD79, CD79b, CD79B gene, CD80, CD95, CD99, CD117, CD122, CDw123, CD134, CDw137, CD158a, CD158b1, CD158b2, CD223, CD276 antigen; clusterin (CLU) gene, Clusterin, c-Met (hepatocyte growth factor receptor (HGFR)), Complement C3, Connective tissue growth factor, COPS signalosome subunit 5, CSF-1 (colony-stimulating factor 1 receptor), CSF2 gene, CTLA-4 (cytotoxic T-lymphocyte protein 4) receptor, C-type lectin domain protein 9A (CLEC9A), Cyclin D1, Cyclin G1, cyclin-dependent kinases (CDK, such as CDK1, CDK12, CDK1B, CDK2-9), cyclooxygenase (such as COX1, COX2), CYP2B1 gene, Cysteine palmitoyltransferase porcupine, Cytochrome P450 11B2, Cytochrome P450 17, cytochrome P450 17A1, Cytochrome P450 2D6, cytochrome P450 3A4, Cytochrome P450 reductase, cytokine signalling-1, cytokine signalling-3, Cytoplasmic isocitrate dehydrogenase, Cytosine deaminase, cytosine DNA methyltransferase, cytotoxic T-lymphocyte protein-4, DDR2 gene, DEAD-box helicase 6 (DDX6), Death receptor 5 (DR5, TRAILR2), Death receptor 4 (DR4, TRAILR1), Delta-like protein ligand (such as 3, 4), Deoxyribonuclease, Deubiquitinating enzymes (DUBs), Dickkopf-1 ligand, dihydrofolate reductase (DHFR), Dihydropyrimidine dehydrogenase, Dipeptidyl peptidase IV, discoidin domain receptor (DDR, such as DDR1), Diacylglycerol kinase zeta (DGKZ), DNA binding protein (such as HU-beta), DNA dependent protein kinase, DNA gyrase, DNA methyltransferase, DNA polymerase (such as alpha), DNA primase, dUTP pyrophosphatase, L-dopachrome tautomerase, E3 ubiquitin-protein ligase (such as RNF128, CBL-B), echinoderm microtubule like protein 4, EGFR tyrosine kinase receptor, Elastase, Elongation factor 1 alpha 2, Elongation factor 2, Endoglin, Endonuclease, endoplasmic reticulum aminopeptidase (ERAP, such as ERAP 1, ERAP2), Endoplasmin, Endosialin, Endostatin, endothelin (such as ET-A, ET-B), Enhancer of zeste homolog 2 (EZH2), Ephrin (EPH) tyrosine kinase (such as Epha3, Ephb4), Ephrin B2 ligand, epidermal growth factor, epidermal growth factor receptors (EGFR), epidermal growth factor receptor (EGFR) gene, Epigen, Epithelial cell adhesion molecule (EpCAM), Erb-b2 (v-erb-b2 avian erythroblastic leukemia viral oncogene homolog 2) tyrosine kinase receptor, Erb-b3 tyrosine kinase receptor, Erb-b4 tyrosine kinase receptor, E-selectin, Estradiol 17 beta dehydrogenase, Estrogen receptor (such as alpha, beta), Estrogen related receptor, Eukaryotic translation initiation factor 5A (EIF5A) gene, Exportin 1, Extracellular signal related kinase (such as 1, 2), Extracellular signal-regulated kinases (ERK), Hypoxia-inducible factor prolyl hydroxylase (HIF-PH or EGLN), Factor (such as Xa, VIIa), farnesoid x receptor (FXR), Fas ligand, Fatty acid synthase (FASN), Ferritin, FGF-2 ligand, FGF-5 ligand, fibroblast growth factor (FGF, such as FGF1, FGF2, FGF4), Fibronectin, focal adhesion kinase (FAK, such as FAK2), folate hydrolase prostate-specific membrane antigen 1 (FOLH1), Folate receptor (such as alpha), Folate, Folate transporter 1, FYN tyrosine kinase, paired basic amino acid cleaving enzyme (FURIN), Beta-glucuronidase, Galactosyltransferase, Galectin-3, Ganglioside GD2, Glucocorticoid, glucocorticoid-induced TNFR-related protein GITR receptor, Glutamate carboxypeptidase II, glutaminase, Glutathione S-transferase P, glycogen synthase kinase (GSK, such as 3-beta), Glypican 3 (GPC3), gonadotropin-releasing hormone (GNRH), Granulocyte macrophage colony stimulating factor (GM-CSF) receptor, Granulocyte-colony stimulating factor (GCSF) ligand, growth factor receptor-bound protein 2 (GRB2), Grp78 (78 kDa glucose-regulated protein) calcium binding protein, molecular chaperone groEL2 gene, Heme oxygenase 1 (HO1), Heme oxygenase 2 (H02), Heat shock protein (such as 27, 70, 90 alpha, beta), Heat shock protein gene, Heat stable enterotoxin receptor, Hedgehog protein, Heparanase, Hepatocyte growth factor, HERV-H LTR associating protein 2, Hexose kinase, Histamine H2 receptor, Histone methyltransferase (DOT1L), histone deacetylase (HDAC, such as 1, 2, 3, 6, 10, 11), Histone H1, Histone H3, HLA class I antigen (A-2 alpha), HLA class II antigen, HLA class I antigen alpha G (HLA-G), Non-classical HLA, Homeobox protein NANOG, HSPB1 gene, Human leukocyte antigen (HLA), Human papillomavirus (such as E6, E7) protein, Hyaluronic acid, Hyaluronidase, Hypoxia inducible factor-1 alpha (HIF1α), Imprinted Maternally Expressed Transcript (H19) gene, mitogen-activated protein kinase 1 (MAP4K1), tyrosine-protein kinase HCK, I-Kappa-B kinase (IKK, such as IKKbe), IL-1 alpha, IL-1 beta, IL-12, IL-12 gene, IL-15, IL-17, IL-2 gene, IL-2 receptor alpha subunit, IL-2, IL-3 receptor, IL-4, IL-6, IL-7, IL-8, immunoglobulin (such as G, G1, G2, K, M), Immunoglobulin Fc receptor, Immunoglobulin gamma Fc receptor (such as I, III, IIIA), indoleamine 2,3-dioxygenase (IDO, such as IDO1 and IDO2), indoleamine pyrrole 2,3-dioxygenase 1 inhibitor, insulin receptor, Insulin-like growth factor (such as 1, 2), Integrin alpha-4/beta-1, integrin alpha-4/beta-7, Integrin alpha-5/beta-1, Integrin alpha-V/beta-3, Integrin alpha-V/beta-5, Integrin alpha-V/beta-6, Intercellular adhesion molecule 1 (ICAM-1), interferon (such as alpha, alpha 2, beta, gamma), Interferon inducible protein absent in melanoma 2 (AIM2), interferon type I receptor, Interleukin 1 ligand, Interleukin 13 receptor alpha 2, interleukin 2 ligand, interleukin-1 receptor-associated kinase 4 (IRAK4), Interleukin-2, Interleukin-29 ligand, Interleukin 35 (IL-35), isocitrate dehydrogenase (such as IDH1, IDH2), Janus kinase (JAK, such as JAK1, JAK2), Jun N terminal kinase, kallikrein-related peptidase 3 (KLK3) gene, Killer cell Ig like receptor, Kinase insert domain receptor (KDR), Kinesin-like protein KIF11, Kirsten rat sarcoma viral oncogene homolog (KRAS) gene, Kisspeptin (KiSS-1) receptor, KIT gene, v-kit Hardy-Zuckerman 4 feline sarcoma viral oncogene homolog (KIT) tyrosine kinase, lactoferrin, Lanosterol-14 demethylase, LDL receptor related protein-1, Leukocyte immunoglobulin-like receptor subfamily B member 1 (ILT2), Leukocyte immunoglobulin-like receptor subfamily B member 2 (ILT4), Leukotriene A4 hydrolase, Listeriolysin, L-Selectin, Luteinizing hormone receptor, Lyase, lymphocyte activation gene 3 protein (LAG-3), Lymphocyte antigen 75, Lymphocyte function antigen-3 receptor, lymphocyte-specific protein tyrosine kinase (LCK), Lymphotactin, Lyn (Lck/Yes novel) tyrosine kinase, lysine demethylases (such as KDM1, KDM2, KDM4, KDM5, KDM6, A/B/C/D), Lysophosphatidate-1 receptor, lysosomal-associated membrane protein family (LAMP) gene, Lysyl oxidase homolog 2, lysyl oxidase protein (LOX), 5-Lipoxygenase (5-LOX), Hematopoietic Progenitor Kinase 1 (HPK1), Hepatocyte growth factor receptor (MET) gene, macrophage colony-stimulating factor (MCSF) ligand, Macrophage migration inhibitory fact, MAGEC1 gene, MAGEC2 gene, Major vault protein, MAPK-activated protein kinase (such as MK2), Mas-related G-protein coupled receptor, matrix metalloprotease (MMP, such as MMP2, MMP9), Mcl-1 differentiation protein, Mdm2 p53-binding protein, Mdm4 protein, Melan-A (MART-1) melanoma antigen, Melanocyte protein Pmel 17, melanocyte stimulating hormone ligand, melanoma antigen family A3 (MAGEA3) gene, Melanoma associated antigen (such as 1, 2, 3, 6), Membrane copper amine oxidase, Mesothelin, MET tyrosine kinase, Metabotropic glutamate receptor 1, Metalloreductase STEAP1 (six transmembrane epithelial antigen of the prostate 1), Metastin, methionine aminopeptidase-2, Methyltransferase, Mitochondrial 3 ketoacyl CoA thiolase, mitogen-activate protein kinase (MAPK), mitogen-activated protein kinase (MEK, such as MEK1, MEK2), mTOR (mechanistic target of rapamycin (serine/threonine kinase), mTOR complex (such as 1,2), mucin (such as 1, 5A, 16), mut T homolog (MTH, such as MTH1), Myc proto-oncogene protein, myeloid cell leukemia 1 (MCL1) gene, myristoylated alanine-rich protein kinase C substrate (MARCKS) protein, NAD ADP ribosyltransferase, natriuretic peptide receptor C, Neural cell adhesion molecule 1, Neurokinin 1 (NK1) receptor, Neurokinin receptor, Neuropilin 2, NF kappa B activating protein, NIMA-related kinase 9 (NEK9), Nitric oxide synthase, NK cell receptor, NK3 receptor, NKG2 A B activating NK receptor, NLRP3 (NACHT LRR PYD domain protein 3) modulators, Noradrenaline transporter, Notch (such as Notch-2 receptor, Notch-3 receptor, Notch-4 receptor), Nuclear erythroid 2-related factor 2, Nuclear Factor (NF) kappa B, Nucleolin, Nucleophosmin, nucleophosmin-anaplastic lymphoma kinase (NPM-ALK), 2 oxoglutarate dehydrogenase, 2,5-oligoadenylate synthetase, O-methylguanine DNA methyltransferase, Opioid receptor (such as delta), Ornithine decarboxylase, Orotate phosphoribosyltransferase, orphan nuclear hormone receptor NR4A1, Osteocalcin, Osteoclast differentiation factor, Osteopontin, OX-40 (tumor necrosis factor receptor superfamily member 4 TNFRSF4, or CD134) receptor, P3 protein, p38 kinase, p38 MAP kinase, p53 tumor suppressor protein, Parathyroid hormone ligand, peroxisome proliferator-activated receptors (PPAR, such as alpha, delta, gamma), P-Glycoprotein (such as 1), phosphatase and tensin homolog (PTEN), phosphatidylinositol 3-kinase (PI3K), phosphoinositide-3 kinase (PI3K such as alpha, delta, gamma), phosphorylase kinase (PK), PKN3 gene, placenta growth factor, platelet-derived growth factor (PDGF, such as alpha, beta), Platelet-derived growth factor (PDGF, such as alpha, beta), Pleiotropic drug resistance transporter, Plexin B1, PLK1 gene, polo-like kinase (PLK), Polo-like kinase 1, Poly (ADP-ribose) polymerase (PARP, such as PARP1, PARP2 and PARP3, PARP7, and mono-PARPs), Preferentially expressed antigen in melanoma (PRAME) gene, Prenyl-binding protein (PrPB), Probable transcription factor PML, Progesterone receptor, Programmed cell death 1 (PD-1), Programmed cell death ligand 1 inhibitor (PD-L1), Prosaposin (PSAP) gene, Prostanoid receptor (EP4), Prostaglandin E2 synthase, prostate specific antigen, Prostatic acid phosphatase, proteasome, Protein E7, Protein farnesyltransferase, protein kinase (PK, such as A, B, C), protein tyrosine kinase, Protein tyrosine phosphatase beta, Proto-oncogene serine/threonine-protein kinase (PIM, such as PIM-1, PIM-2, PIM-3), P-Selectin, Purine nucleoside phosphorylase, purinergic receptor P2X ligand gated ion channel 7 (P2X7), Pyruvate dehydrogenase (PDH), Pyruvate dehydrogenase kinase, Pyruvate kinase (PYK), 5-Alpha-reductase, Raf protein kinase (such as 1, B), RAF1 gene, Ras gene, Ras GTPase, RET gene, Ret tyrosine kinase receptor, retinoblastoma associated protein, retinoic acid receptor (such as gamma), Retinoid X receptor, Rheb (Ras homolog enriched in brain) GTPase, Rho (Ras homolog) associated protein kinase 2, ribonuclease, Ribonucleotide reductase (such as M2 subunit), Ribosomal protein S6 kinase, RNA polymerase (such as I, II), Ron (Recepteur d'Origine Nantais) tyrosine kinase, ROS1 (ROS proto-oncogene 1, receptor tyrosine kinase) gene, Ros1 tyrosine kinase, Runt-related transcription factor 3, Gamma-secretase, S100 calcium binding protein A9, Sarco endoplasmic calcium ATPase, Second mitochondria-derived activator of caspases (SMAC) protein, Secreted frizzled related protein-2, Secreted phospholipase A2, Semaphorin-4D, Serine protease, serine/threonine kinase (STK), serine/threonine-protein kinase (TBK, such as TBK1), signal transduction and transcription (STAT, such as STAT-1, STAT-3, STAT-5), Signaling lymphocytic activation molecule (SLAM) family member 7, six-transmembrane epithelial antigen of the prostate (STEAP) gene, SL cytokine ligand, smoothened (SMO) receptor, Sodium iodide cotransporter, Sodium phosphate cotransporter 2B, Somatostatin receptor (such as 1, 2, 3, 4, 5), Sonic hedgehog protein, Son of sevenless (SOS), Specific protein 1 (Sp1) transcription factor, Sphingomyelin synthase, Sphingosine kinase (such as 1, 2), Sphingosine-1-phosphate receptor-1, spleen tyrosine kinase (SYK), SRC gene, Src tyrosine kinase, Stabilin-1 (STAB1), STAT3 gene, Steroid sulfatase, Stimulator of interferon genes (STING) receptor, stimulator of interferon genes protein, Stromal cell-derived factor 1 ligand, SUMO (small ubiquitin-like modifier), Superoxide dismutase, Suppressor of cytokine signaling modulators (SOCS), Survivin protein, Synapsin 3, Syndecan-1, Synuclein alpha, T cell surface glycoprotein CD28, tank-binding kinase (TBK), TATA box-binding protein-associated factor RNA polymerase I subunit B (TAF1B) gene, T-cell CD3 glycoprotein zeta chain, T-cell differentiation antigen CD6, T-cell immunoglobulin and mucin-domain containing-3 (TIM-3), T-cell surface glycoprotein CD8, Tec protein tyrosine kinase, Tek tyrosine kinase receptor, telomerase, Telomerase reverse transcriptase (TERT) gene, Tenascin, Three prime repair exonuclease 1 (TREX1), Three prime repair exonuclease 2 (TREX2), Thrombopoietin receptor, Thymidine kinase, Thymidine phosphorylase, Thymidylate synthase, Thymosin (such as alpha 1), Thyroid hormone receptor, Thyroid stimulating hormone receptor, Tissue factor, TNF related apoptosis inducing ligand, TNFR1 associated death domain protein, TNF-related apoptosis-inducing ligand (TRAIL) receptor, TNFSF11 gene, TNFSF9 gene, Toll-like receptor (TLR such as 1-13), topoisomerase (such as I, II, III), Transcription factor, Transferase, transferrin (TF), transforming growth factor alpha (TGFα), transforming growth factor beta (TGFB) and isoforms thereof, TGF beta 2 ligand, Transforming growth factor TGF-β receptor kinase, Transglutaminase, Translocation associated protein, Transmembrane glycoprotein NMB, Trop-2 calcium signal transducer, trophoblast glycoprotein (TPBG) gene, Trophoblast glycoprotein, Tropomyosin receptor kinase (Trk) receptor (such as TrkA, TrkB, TrkC), tryptophan 2,3-dioxygenase (TDO), Tryptophan 5-hydroxylase, Tubulin, Tumor necrosis factor (TNF, such as alpha, beta), Tumor necrosis factor 13C receptor, tumor progression locus 2 (TPL2), Tumor protein 53 (TP53) gene, Tumor suppressor candidate 2 (TUSC2) gene, Tumor specific neoantigens, Tyrosinase, Tyrosine hydroxylase, tyrosine kinase (TK), Tyrosine kinase receptor, Tyrosine kinase with immunoglobulin-like and EGF-like domains (TIE) receptor, Tyrosine protein kinase ABL1 inhibitor, Ubiquitin, Ubiquitin carboxyl hydrolase isozyme L5, Ubiquitin thioesterase-14, Ubiquitin-conjugating enzyme E2I (UBE2I, UBC9), Ubiquitin-specific-processing protease 7 (USP7), Urease, Urokinase plasminogen activator, Uteroglobin, Vanilloid VR1, Vascular cell adhesion protein 1, vascular endothelial growth factor receptor (VEGFR), V-domain Ig suppressor of T-cell activation (VISTA), VEGF-1 receptor, VEGF-2 receptor, VEGF-3 receptor, VEGF-A, VEGF-B, Vimentin, Vitamin D3 receptor, Proto-oncogene tyrosine-protein kinase, Mer (Mer tyrosine kinase receptor modulators), YAP (Yes-associated protein modulators)es, Wee-1 protein kinase, Werner Syndrome RecQ Like Helicase (WRN), Wilms' tumor antigen 1, Wilms' tumor protein, WW domain containing transcription regulator protein 1 (TAZ), X-linked inhibitor of apoptosis protein, Zinc finger protein transcription factor or any combination thereof.

In various embodiments, the agent that inhibits binding between CD47 and SIRPα (e.g., magrolimab); and the NAE1 inhibitor (e.g., pevonedistat), as described herein, is combined with one or more additional therapeutic agents that may be categorized by their mechanism of action into, for example, the following groups: anti-metabolites/anti-cancer agents, such as pyrimidine analogs floxuridine, capecitabine, cytarabine, CPX-351 (liposomal cytarabine, daunorubicin), and TAS-118; Alpha 1 adrenoceptor/Alpha 2 adrenoceptor antagonists, such as phenoxybenzamine hydrochloride (injectable, pheochromocytoma); Androgen receptor antagonists, such as nilutamide; anti-cadherin antibodies, such as HKT-288; anti-leucine-rich repeat containing 15 (LRRC15) antibodies, such as ABBV-085. ARGX-110; angiotensin receptor blockers, nitric oxide donors; antisense oligonucleotides, such as AEG35156, IONIS-KRAS-2.5Rx, EZN-3042, RX-0201, IONIS-AR-2.5Rx, BP-100 (prexigebersen), IONIS-STAT3-2.5Rx; anti-angiopoietin (ANG)-2 antibodies, such as MEDI3617, and LY3127804; anti-ANG-1/ANG-2 antibodies, such as AMG-780; anti-CSF1R antibodies, such as emactuzumab, LY3022855, AMG-820, FPA-008 (cabiralizumab); anti-endoglin antibodies, such as TRC105 (carotuximab); anti-ERBB antibodies, such as CDX-3379, HLX-02, seribantumab; anti-HER2 antibodies, such as HERCEPTIN® (trastuzumab), trastuzumab biosimimar, margetuximab, MEDI4276, BAT-8001, Pertuzumab (Perjeta), RG6264, ZW25 (a bispecific HER2-directed antibody targeting the extracellular domains 2 and 4; Cancer Discov. 2019 January; 9(1):8; PMID: 30504239); anti-HLA-DR antibodies, such as IMMU-114; anti-IL-3 antibodies, such as JNJ-56022473; anti-TNF receptor superfamily member 18 (TNFRSF18, GITR; NCBI Gene ID: 8784) antibodies, such as MK-4166, MEDI1873, FPA-154, INCAGN-1876, TRX-518, BMS-986156, MK-1248, GWN-323; and those described, e.g., in Intl. Patent Publ. Nos. WO 2017/096179, WO 2017/096276, WO 2017/096189; and WO 2018/089628; anti-EphA3 antibodies, such as KB-004; anti-CD37 antibodies, such as otlertuzumab (TRU-016); anti-FGFR-3 antibodies, such as LY3076226, B-701; anti-FGFR-2 antibodies, such as GAL-F2; anti-05 antibodies, such as ALXN-1210; anti-EpCAM antibodies, such as VB4-845; anti-CEA antibodies, such as RG-7813; anti-Carcinoembryonic-antigen-related-cell-adhesion-molecule-6 (CEACAM6, CD66C) antibodies, such as BAY-1834942, NEO-201 (CEACAM 5/6); anti-GD2 antibodies, such as APN-301; anti-interleukin-17 (IL-17) antibodies, such as CJM-112; anti-interleukin-1 beta antibodies, such as canakinumab (ACZ885), VPM087; anti-carbonic anhydrase 9 (CA9, CAIX) antibodies, such as TX-250; anti-Mucin 1 (MUC1) antibodies, such as gatipotuzumab, Mab-AR-20.5; anti-KMA antibodies, such as MDX-1097; anti-CD55 antibodies, such as PAT-SC1; anti-c-Met antibodies, such as ABBV-399; anti-PSMA antibodies, such as ATL-101; anti-CD100 antibodies, such as VX-15; anti-EPHA3 antibodies, such as fibatuzumab; anti-APRIL antibodies, such as BION-1301; anti-fibroblast activation protein (FAP)/IL-2R antibodies, such as RG7461; anti-fibroblast activation protein (FAP)/TRAIL-R2 antibodies, such as RG7386; anti-fucosyl-GM1 antibodies, such as BMS-986012; anti-IL-8 (Interleukin-8) antibodies, such as HuMax-Inflam; anti-myostatin inhibitors, such as landogrozumab; anti-delta-like protein ligand 3 (DDL3) antibodies, such as rovalpituzumab tesirine; anti-DLL4 (delta like ligand 4) antibodies, such as demcizumab; anti-clusterin antibodies, such as AB-16B5; anti-Ephrin-A4 (EFNA4) antibodies, such as PF-06647263; anti-mesothelin antibodies, such as BMS-986148, Anti-MSLN-MMAE; anti-sodium phosphate cotransporter 2B (NaP2B) antibodies, such as lifastuzumab; anti-TGFβ antibodies, such as SAR439459; anti-transforming growth factor-beta (TGF-beta) antibodies, such as ABBV-151, LY3022859, NIS793, XOMA 089; purine analogs, folate antagonists (such as pralatrexate), cladribine, pentostatin, fludarabine and related inhibitors; antiproliferative/antimitotic agents including natural products, such as vinca alkaloids (vinblastine, vincristine) and microtubule disruptors such as taxane (paclitaxel, docetaxel), vinblastin, nocodazole, epothilones, vinorelbine (NAVELBINE®), and epipodophyllotoxins (etoposide, teniposide); DNA damaging agents, such as actinomycin, amsacrine, busulfan, carboplatin, chlorambucil, cisplatin, cyclophosphamide (CYTOXAN®), dactinomycin, daunorubicin, doxorubicin, DEBDOX, epirubicin, iphosphamide, melphalan, merchlorethamine, mitomycin C, mitoxantrone, nitrosourea, procarbazine, taxol, Taxotere, teniposide, etoposide, and triethylenethiophosphoramide; DNA-hypomethylating agents, such as guadecitabine (SGI-110), oral decitabine and cedazuridine (ASTX727); antibiotics such as dactinomycin, daunorubicin, doxorubicin, idarubicin, anthracyclines, mitoxantrone, bleomycins, plicamycin (mithramycin); enzymes such as L-asparaginase which systemically metabolizes L-asparagine and deprives cells which do not have the capacity to synthesize their own asparagine; DNAi oligonucleotides targeting Bcl-2, such as PNT2258; agents that activate or reactivate latent human immunodeficiency virus (HIV), such as panobinostat and romidepsin; asparaginase stimulators, such as crisantaspase (Erwinase®) and GRASPA (ERY-001, ERY-ASP), calaspargase pegol, pegaspargase; pan-Trk, ROS1 and ALK inhibitors, such as entrectinib, TPX-0005; anaplastic lymphoma kinase (ALK) inhibitors, such as alectinib, ceritinib, alecensa (RG7853), ALUNBRIG® (brigatinib); antiproliferative/antimitotic alkylating agents, such as nitrogen mustard cyclophosphamide and analogs (e.g., melphalan, chlorambucil, hexamethylmelamine, thiotepa), alkyl nitrosoureas (e.g., carmustine) and analogs, streptozocin, and triazenes (e.g., dacarbazine); antiproliferative/antimitotic antimetabolites, such as folic acid analogs (methotrexate); platinum coordination complexes (e.g., cisplatin, oxiloplatinim, and carboplatin), procarbazine, hydroxyurea, mitotane, and aminoglutethimide; hormones, hormone analogs (e.g., estrogen, tamoxifen, goserelin, bicalutamide, and nilutamide), and aromatase inhibitors (e.g., letrozole and anastrozole); antiplatelet agents; anticoagulants such as heparin, synthetic heparin salts, and other inhibitors of thrombin; fibrinolytic agents such as tissue plasminogen activator, streptokinase, urokinase, aspirin, dipyridamole, ticlopidine, and clopidogrel; antimigratory agents; antisecretory agents (e.g., breveldin); immunosuppressives, such as tacrolimus, sirolimus, azathioprine, and mycophenolate; growth factor inhibitors, and vascular endothelial growth factor inhibitors; fibroblast growth factor inhibitors, such as FPA14; AMP activated protein kinase stimulators, such as metformin hydrochloride; ADP ribosyl cyclase-1 inhibitors, such as daratumumab (DARZALEX®); Caspase recruitment domain protein-15 stimulators, such as mifamurtide (liposomal); CCR5 chemokine antagonists, such as MK-7690 (vicriviroc); CDC7 protein kinase inhibitors, such as TAK-931; Cholesterol side-chain cleavage enzyme inhibitors, such as ODM-209; Dihydropyrimidine dehydrogenase/Orotate phosphoribosyltransferase inhibitors, such as Cefesone (tegafur+gimeracil+oteracil potassium); DNA polymerase/Ribonucleotide reductase inhibitors, such as clofarabine; DNA interference oligonucleotides, such as PNT2258, AZD-9150; Estrogen receptor modulators, such as bazedoxifene; Estrogen receptor agonists/Progesterone receptor antagonists, such as TRI-CYCLEN LO (norethindrone+ethinyl estradiol); HLA class I antigen A-2 alpha modulators, such as FH-MCVA2TCR; HLA class I antigen A-2 alpha/MART-1 melanoma antigen modulators, such as MART-1 F5 TCR engineered PBMC; Human Granulocyte Colony Stimulating Factors, such as PF-06881894; GNRH receptor agonists, such as leuprorelin acetate, leuprorelin acetate sustained release depot (ATRIGEL), triptorelin pamoate, goserelin acetate; GNRH receptor antagonists, such as elagolix, relugolix, degarelix; Endoplasmin modulators, such as anlotinib; H+K+ ATPase inhibitors, such as omeprazole, esomeprazole; ICAM-1/CD55 modulators, such as cavatak (V-937); IL-15/IL-12 modulators, such as SAR441000; Interleukin 23A inhibitors, such as guselkumab; Lysine specific histone demethylase 1 inhibitors, such as CC-90011; IL-12 Mrna, such as MEDI1191; RIG-I modulators, such as RGT-100; NOD2 modulators, such as SB-9200, and IR-103; Progesterone receptor agonists, such as levonorgestrel; Protein cereblon modulators, such as CC-92480, CC-90009; Protein cereblon modulators/DNA binding protein Ikaros inhibitors/Zinc finger binding protein Aiolos inhibitors, such as iberdomide; Retinoid X receptor modulators, such as alitretinoin, bexarotene (oral formulation); RIP-1 kinase inhibitors, such as GSK-3145095; selective oestrogen receptor degraders, such as AZD9833; SUMO inhibitors, such as TAK-981; Thrombopoietin receptor agonists, such as eltrombopag; Thyroid hormone receptor agonists, such as levothyroxine sodium; TNF agonists, such as tasonermin; Tyrosine phosphatase substrate 1 inhibitors, such as CC-95251; HER2 inhibitors, such as neratinib, tucatinib (ONT-380); EGFR/ErbB2/Ephb4 inhibitors, such as tesevatinib; EGFR/HER2 inhibitors, such as TAK-788; EGFR family tyrosine kinase receptor inhibitors, such as DZD-9008; EGFR/ErbB-2 inhibitors, such as varlitinib; mutant selective EGFR inhibitors, such as PF-06747775, EGF816 (nazartinib), ASP8273, ACEA-0010, BI-1482694; epha2 inhibitors, such as MM-310; polycomb protein (EED) inhibitors, such as MAK683; DHFR inhibitor/Folate transporter 1 modulator/Folate receptor antagonist, such as pralatrexate; DHFR/GAR transformylase/Thymidylate synthase/Transferase inhibitors, such as pemetrexed disodium; p38 MAP kinase inhibitors, such as ralimetinib; PRMT inhibitors, such as MS203, PF-06939999, GSK3368715, GSK3326595; Sphingosine kinase 2 (SK2) inhibitors, such as opaganib; Nuclear erythroid 2-related factor 2 stimulators, such as omaveloxolone (RTA-408); Tropomyosin receptor kinase (TRK) inhibitors, such as LOXO-195, ONO-7579; Mucin 1 inhibitors, such as GO-203-2C; MARCKS protein inhibitors, such as BIO-11006; Folate antagonists, such as arfolitixorin; Galectin-3 inhibitors, such as GR-MD-02; Phosphorylated P68 inhibitors, such as RX-5902; CD95/TNF modulators, such as ofranergene obadenovec; pan-PIM kinase inhibitors, such as INCB-053914; IL-12 gene stimulators, such as EGEN-001, tavokinogene telseplasmid; Heat shock protein HSP90 inhibitors, such as TAS-116, PEN-866; VEGF/HGF antagonists, such as MP-0250; VEGF ligand inhibitors, such as bevacizumab biosimilar; VEGF receptor antagonists/VEGF ligand inhibitors, such as ramucirumab; VEGF-1/VEGF-2/VEGF-3 receptor antagonists; such as fruquintinib; VEGF-1/VEGF-2 receptor modulators, such as HLA-A2402/HLA-A0201 restricted epitope peptide vaccine; Placenta growth factor ligand inhibitor/VEGF-A ligand inhibitor, such as aflibercept; SYK tyrosine kinase/JAK tyrosine kinase inhibitors, such as ASN-002; Trk tyrosine kinase receptor inhibitors, such as larotrectinib sulfate; JAK3/JAK1/TBK1 kinase inhibitors, such as CS-12912; IL-24 antagonist, such as AD-IL24; NLRP3 (NACHT LRR PYD domain protein 3) modulators, such as BMS-986299; RIG-I agonists, such as RGT-100; Aerolysin stimulators, such as topsalysin; P-Glycoprotein 1 inhibitors, such as HM-30181A; CSF-1 antagonists, such as ARRY-382, BLZ-945; CCR8 inhibitors, such as JTX-1811, 1-309, SB-649701, HG-1013, RAP-310; anti-Mesothelin antibodies, such as SEL-403; Thymidine kinase stimulators, such as aglatimagene besadenovec; Polo-like kinase 1 inhibitors, such as PCM-075, onvansertib; NAE inhibitors, such as pevonedistat (MLN-4924), TAS-4464; Pleiotropic pathway modulators, such as avadomide (CC-122); Amyloid protein binding protein-1 inhibitorS/Ubiquitin ligase modulators, such as pevonedistat; FoxM1 inhibitors, such as thiostrepton; UBA1 inhibitors, such as TAK-243; Src tyrosine kinase inhibitors, such as VAL-201; VDAC/HK inhibitors, such as VDA-1102; Elf4a inhibitors, such as rohinitib, eFT226; TP53 gene stimulators, such as ad-p53; Retinoic acid receptor agonists, such as tretinoin; Retinoic acid receptor alpha (RARα) inhibitors, such as SY-1425; SIRT3 inhibitors, such as YC8-02; Stromal cell-derived factor 1 ligand inhibitors, such as olaptesed pegol (NOX-A12); IL-4 receptor modulators, such as MDNA-55; Arginase-I stimulators, such as pegzilarginase; Topoisomerase I inhibitors, such as irinotecan hydrochloride, Onivyde; Topoisomerase I inhibitor/hypoxia inducible factor-1 alpha inhibitors, such as PEG-SN38 (firtecan pegol); Hypoxia inducible factor-1 alpha inhibitors, such as PT-2977, PT-2385; CD122 (IL-2 receptor) agonists, such as proleukin (aldesleukin, IL-2); pegylated IL-2 (eg NKTR-214); modified variants of IL-2 (eg THOR-707); TLR7/TLR8 agonist, such as NKTR-262; TLR7 agonists, such as DS-0509, GS-9620, LHC-165, TMX-101 (imiquimod); p53 tumor suppressor protein stimulators such as kevetrin; Mdm4/Mdm2 p53-binding protein inhibitors, such as ALRN-6924; kinesin spindle protein (KSP) inhibitors, such as filanesib (ARRY-520); CD80-Fc fusion protein inhibitors, such as FPT-155; Menin and mixed lineage leukemia (MLL) inhibitors such as KO-539; Liver x receptor agonists, such as RGX-104; IL-10 agonists, such as Pegilodecakin (AM-0010); VEGFR/PDGFR inhibitors, such as vorolanib; IRAK4 inhibitors, such as CA-4948; anti-TLR-2 antibodies, such as OPN-305; Calmodulin modulators, such as CBP-501.

Glucocorticoid receptor antagonists, such as relacorilant (CORT-125134); Second mitochondria-derived activator of caspases (SMAC) protein inhibitors, such as BI-891065; Lactoferrin modulators, such as LTX-315; KIT proto-oncogene, receptor tyrosine kinase (KIT) inhibitors, such as PLX-9486; platelet derived growth factor receptor alpha (PDGFRA)/KIT proto-oncogene, receptor tyrosine kinase (KIT) mutant-specific antagonists/inhibitors such as BLU-285, DCC-2618; Exportin 1 inhibitors, such as eltanexor; CHST15 gene inhibitors, such as STNM-01; Somatostatin receptor antagonist, such as OPS-201; CEBPA gene stimulators, such as MTL-501; DKK3 gene modulators, such as MTG-201; Chemokine (CXCR1/CXCR2) inhibitors, such as SX-682; p70s6k inhibitors, such as MSC2363318A; methionine aminopeptidase 2 (MetAP2) inhibitors, such as M8891, APL-1202; arginine N-methyltransferase 5 inhibitors, such as GSK-3326595; CD71 modulators, such as CX-2029 (ABBV-2029); ATM (ataxia telangiectasia) inhibitors, such as AZD0156, AZD1390; CHK1 inhibitors, such as GDC-0575, LY2606368 (prexasertib), SRA737, RG7741 (CHK1/2); CXCR4 antagonists, such as BL-8040, LY2510924, burixafor (TG-0054), X4P-002, X4P-001-IO, Plerixafor; EXH2 inhibitors, such as GSK2816126; KDM1 inhibitors, such as ORY-1001, IMG-7289, INCB-59872, GSK-2879552; CXCR2 antagonists, such as AZD-5069; DNA dependent protein kinase inhibitors, such as MSC2490484A (nedisertib), VX-984, AsiDNA (DT-01); protein kinase C (PKC) inhibitors, such as LXS-196, sotrastaurin; selective estrogen receptor downregulators (SERD), such as fulvestrant (Faslodex®), RG6046, RG6047, RG6171, elacestrant (RAD-1901), SAR439859 and AZD9496; selective estrogen receptor covalent antagonists (SERCAs), such as H3B-6545; selective androgen receptor modulator (SARM), such as GTX-024, darolutamide; transforming growth factor-beta (TGF-beta) kinase antagonists, such as galunisertib, LY3200882; TGF-beta inhibitors described in WO 2019/103203; TGF beta receptor 1 inhibitors, such as PF-06952229; bispecific antibodies, such as ABT-165 (DLL4/VEGF), MM-141 (IGF-1/ErbB3), MM-111 (Erb2/Erb3), JNJ-64052781 (CD19/CD3), PRS-343 (CD-137/HER2), AFM26 (BCMA/CD16A), JNJ-61186372 (EGFR/cMET), AMG-211 (CEA/CD3), RG7802 (CEA/CD3), ERY-974 (CD3/GPC3) vancizumab (angiopoietins/VEGF), PF-06671008 (Cadherins/CD3), AFM-13 (CD16/CD30), APV0436 (CD123/CD3), flotetuzumab (CD123/CD3), REGN-1979 (CD20/CD3), MCLA-117 (CD3/CLEC12A), MCLA-128 (HER2/HER3), JNJ-0819, JNJ-7564 (CD3/heme), AMG-757 (DLL3-CD3), MGD-013 (PD-1/LAG-3), FS-118 (LAG-3/PD-L1) MGD-019 (PD-1/CTLA-4), KN-046 (PD-1/CTLA-4), MEDI-5752 (CTLA-4/PD-1), RO-7121661 (PD-1/TIM-3), XmAb-20717 (PD-1/CTLA-4), AK-104 (CTLA-4/PD-1), AMG-420 (BCMA/CD3), BI-836880 (VEFG/ANG2), JNJ-63709178 (CD123/CD3), MGD-007 (CD3/gpA33), MGD-009 (CD3/B7H3), AGEN1223, IMCgp100 (CD3/gp100), AGEN-1423, ATOR-1015 (CTLA-4/OX40), LY-3415244 (TIM-3/PDL1), INHIBRX-105 (4-1BB/PDL1), faricimab (VEGF-A/ANG-2), FAP-4-IBBL (4-1BB/FAP), XmAb-13676 (CD3/CD20), TAK-252 (PD-1/OX40L), TG-1801 (CD19/CD47), XmAb-18087 (SSTR2/CD3), catumaxomab (CD3/EpCAM), SAR-156597 (IL4/IL13), EMB-01 (EGFR/cMET), REGN-4018 (MUC16/CD3), REGN-1979 (CD20/CD3), RG-7828 (CD20/CD3), CC-93269 (CD3/BCMA), REGN-5458 (CD3/BCMA), navicixizumab (DLL4/VEGF), GRB-1302 (CD3/Erbb2), vanucizumab (VEGF-A/ANG-2), GRB-1342 (CD38/CD3), GEM-333 (CD3/CD33), IMM-0306 (CD47/CD20), RG6076, MEDI5752 (PD-1/CTLA-4), LY3164530 (MET/EGFR); Alpha-ketoglutarate dehydrogenase (KGDH) inhibitors, such as CPI-613; XPO1 inhibitors, such as selinexor (KPT-330); Isocitrate dehydrogenase 2 (IDH2) inhibitors, such as enasidenib (AG-221); IDH1 inhibitors such as AG-120, and AG-881 (IDH1 and IDH2), IDH-305, BAY-1436032; IDH1 gene inhibitors, such as ivosidenib; interleukin-3 receptor (IL-3R) modulators, such as SL-401; Arginine deiminase stimulators, such as pegargiminase (ADI-PEG-20); claudin-18 inhibitors, such as claudiximab; β-catenin inhibitors, such as CWP-291; chemokine receptor 2 (CCR) inhibitors, such as PF-04136309, CCX-872, BMS-813160 (CCR2/CCR5); thymidylate synthase inhibitors, such as ONX-0801; ALK/ROS1 inhibitors, such as lorlatinib; tankyrase inhibitors, such as G007-LK; triggering receptor expressed on myeloid cells 1 (TREM1; NCBI Gene ID: 54210), such as PY159; triggering receptor expressed on myeloid cells 2 (TREM2; NCBI Gene ID: 54209), such as PY314; Mdm2 p53-binding protein inhibitors, such as CMG-097, HDM-201; c-PIM inhibitors, such as PIM447; sphingosine kinase-2 (SK2) inhibitors, such as Yeliva® (ABC294640); DNA polymerase inhibitors, such as sapacitabine; Cell cycle/Microtubule inhibitors, such as eribulin mesylate; c-MET inhibitors, such as AMG-337, savolitinib, tivantinib (ARQ-197), capmatinib, and tepotinib, ABT-700, AG213, AMG-208, JNJ-38877618 (OMO-1), merestinib, HQP-8361; c-Met/VEGFR inhibitors, such as BMS-817378, TAS-115; c-Met/RON inhibitors, such as BMS-777607; BCR/ABL inhibitors, such as rebastinib, asciminib, ponatinib (ICLUSIG®); MNK1/MNK2 inhibitors, such as eFT-508; Cytochrome P450 11B2/Cytochrome P450 17/AKT protein kinase inhibitors, such as LAE-201; Cytochrome P450 3A4 stimulators, such as mitotane; lysine-specific demethylase-1 (LSD1) inhibitors, such as CC-90011; CSF1R/KIT and FLT3 inhibitors, such as pexidartinib (PLX3397); Flt3 tyrosine kinase/Kit tyrosine kinase inhibitor and PDGF receptor antagonists, such as quizartinib dihydrochloride; kinase inhibitors, such as vandetanib; E selectin antagonists, such as GMI-1271; differentiation inducers, such as tretinoin; epidermal growth factor receptor (EGFR) inhibitors, such as osimertinib (AZD-9291), cetuximab; topoisomerase inhibitors, such as Adriamycin, doxorubicin, daunorubicin, dactinomycin, DaunoXome, Caelyx, eniposide, epirubicin, etoposide, idarubicin, irinotecan, mitoxantrone, pixantrone, sobuzoxane, topotecan, irinotecan, MINI-398 (liposomal irinotecan), vosaroxin and GPX-150, aldoxorubicin, AR-67, mavelertinib, AST-2818, avitinib (ACEA-0010), irofulven (MGI-114); corticosteroids, such as cortisone, dexamethasone, hydrocortisone, methylprednisolone, prednisone, prednisolone; growth factor signal transduction kinase inhibitors; nucleoside analogs, such as DFP-10917; Axl inhibitors, such as BGB-324 (bemcentinib), SLC-0211; Axl/Flt3 inhibitors, such as gilteritinib; Inhibitors of bromodomain and extraterminal motif (BET) proteins, including ABBV-744, BRD2 (NCBI Gene ID: 6046), BRD3 (NCBI Gene ID: 8019), BRD4 (NCBI Gene ID: 23476), and bromodomain testis-specific protein (BRDT; NCBI Gene ID: 676), such as INCB-054329, INCB057643, TEN-010, AZD-5153, ABT-767, BMS-986158, CC-90010, GSK525762 (molibresib), NHWD-870, ODM-207, GSK-2820151, GSK-1210151A, ZBC246, ZBC260, ZEN3694, FT-1101, RG-6146, CC-90010, CC-95775, mivebresib, BI-894999, PLX-2853, PLX-51107, CPI-0610, GS-5829; PARP inhibitors, such as olaparib (MK7339), rucaparib, veliparib, talazoparib, ABT-767, BGB-290, fluzolepali (SHR-3162), niraparib (JNJ-64091742), bendamustine hydrochloride; PARP/Tankyrase inhibitors such as 2X-121 (e-7499); IMP-4297, SC-10914, IDX-1197, HWH-340, CK-102, simmiparib; Proteasome inhibitors, such as ixazomib (NINLARO®), carfilzomib (Kyprolis®), marizomib, bortezomib; Glutaminase inhibitors, such as CB-839 (telaglenastat), bis-2-(5-phenylacetamido-1,3,4-thiadiazol-2-yl)ethyl sulfide (BPTES); mitochondrial complex I inhibitors, such as metformin, phenformin; vaccines, such as peptide vaccine TG-01 (RAS), GALE-301, GALE-302, nelipepimut-s, SurVaxM, DSP-7888, TPIV-200, PVX-410, VXL-100, DPX-E7, ISA-101, 6MHP, OSE-2101, galinpepimut-S, SVN53-67/M57-KLH, IMU-131, peptide subunit vaccine (acute lymphoblastic leukemia, University Children's Hospital Tuebingen); bacterial vector vaccines such as CRS-207/GVAX, axalimogene filolisbac (ADXS11-001); adenovirus vector vaccines such as nadofaragene firadenovec; autologous Gp96 vaccine; dendritic cells vaccines, such as CVactm, stapuldencel-T, eltrapuldencel-T, rocapuldencel-T (AGS-003), DCVAC, SL-701, BSK01TM, ADXS31-142, autologous dendritic cell vaccine (metastatic malignant melanoma, intradermal/intravenous, Universitatsklinikum Erlangen); oncolytic vaccines such as, talimogene laherparepvec, pexastimogene devacirepvec, GL-ONC1, MG1-MA3, parvovirus H-1, ProstAtak, enadenotucirev, MG1MA3, ASN-002 (TG-1042); therapeutic vaccines, such as CVAC-301, CMP-001, CreaVax-BC, PF-06753512, VBI-1901, TG-4010, ProscaVax™; tumor cell vaccines, such as Vigil® (IND-14205), Oncoquest-L vaccine; live attenuated, recombinant, serotype 1 poliovirus vaccine, such as PVS-RIPO; Adagloxad simolenin; MEDI-0457; DPV-001 a tumor-derived, autophagosome enriched cancer vaccine; RNA vaccines such as, CV-9209, LV-305; DNA vaccines, such as MEDI-0457, MVI-816, INO-5401; modified vaccinia virus Ankara vaccine expressing p53, such as MVA-p53; DPX-Survivac; BriaVax™; GI-6301; GI-6207; GI-4000; 10-103; Neoantigen peptide vaccines, such as AGEN-2017, GEN-010, NeoVax, RG-6180, GEN-009, PGV-001 (TLR-3 agonist), GRANITE-001, NEO-PV-01; Peptide vaccines that target heat shock proteins, such as PhosphoSynVax™; Vitespen (HSPPC-96-C), NANT Colorectal Cancer Vaccine containing aldoxorubicin, autologous tumor cell vaccine+systemic CpG-B+IFN-alpha (cancer), IO-120+IO-103 (PD-L1/PD-L2 vaccines), HB-201, HB-202, HB-301, TheraT®*-based vaccines; TLR-3 agonist/interferon inducers, such as Poly-ICLC (NSC-301463); STAT-3 inhibitors, such as napabucasin (BBI-608); ATPase p97 inhibitors, such as CB-5083; smoothened (SMO) receptor inhibitors, such as Odomzo® (sonidegib, formerly LDE-225), LEQ506, vismodegib (GDC-0449), BMS-833923, glasdegib (PF-04449913), LY2940680, and itraconazole; interferon alpha ligand modulators, such as interferon alpha-2b, interferon alpha-2a biosimilar (Biogenomics), ropeginterferon alfa-2b (AOP-2014, P-1101, PEG IFN alpha-2b), Multiferon (Alfanative, Viragen), interferon alpha 1b, Roferon-A (Canferon, Ro-25-3036), interferon alfa-2a follow-on biologic (Biosidus)(Inmutag, Inter 2A), interferon alfa-2b follow-on biologic (Biosidus-Bioferon, Citopheron, Ganapar, Beijing Kawin Technology—Kaferon), Alfaferone, pegylated interferon alpha-1b, peginterferon alfa-2b follow-on biologic (Amega), recombinant human interferon alpha-1b, recombinant human interferon alpha-2a, recombinant human interferon alpha-2b, veltuzumab-IFN alpha 2b conjugate, Dynavax (SD-101), and interferon alfa-n1 (Humoferon, SM-10500, Sumiferon); interferon gamma ligand modulators, such as interferon gamma (OH-6000, Ogamma 100); telomerase modulators, such as, tertomotide (GV-1001, HR-2802, Riavax) and imetelstat (GRN-163, JNJ-63935937); DNA methyltransferases inhibitors, such as temozolomide (CCRG-81045), decitabine, oral decitabine and cedazuridine (ASTX727), guadecitabine (S-110, SGI-110), KRX-0402, RX-3117, RRx-001, and azacytidine (CC-486); DNA gyrase inhibitors, such as pixantrone and sobuzoxane; DNA gyrase inhibitors/Topoisimerase II inhibitors, such as amrubicin; Bcl-2 family protein inhibitors, such as ABT-263, venetoclax (ABT-199), obatoclax mesylate, pelcitoclax, ABT-737, RG7601, and AT-101; Bcl-2/Bcl-XL inhibitors, such as navitoclax (ABT-263; RG-7433); Notch inhibitors, such as LY3039478 (crenigacestat), tarextumab (anti-Notch2/3), BMS-906024; hyaluronidase stimulators, such as PEGPH-20; Erbb2 tyrosine kinase receptor inhibitors/Hyaluronidase stimulators, such as Herceptin Hylecta; Wnt pathway inhibitors, such as SM-04755, PRI-724, WNT-974; gamma-secretase inhibitors, such as PF-03084014, MK-0752, RO-4929097; Grb-2 (growth factor receptor bound protein-2) inhibitors, such as BP1001; TRAIL pathway-inducing compounds, such as ONC201, ABBV-621; TRAIL modulators, such as SCB-313; Focal adhesion kinase inhibitors, such as VS-4718, defactinib, GSK2256098; hedgehog inhibitors, such as saridegib, sonidegib (LDE225), glasdegib; Aurora kinase inhibitors, such as alisertib (MLN-8237), and AZD-2811, AMG-900, barasertib, ENMD-2076; HSPB1 modulators (heat shock protein 27, HSP27), such as brivudine, apatorsen; ATR inhibitors, such as BAY-937, AZD6738, AZD6783, VX-803, VX-970 (berzosertib) and VX-970; Hsp90 inhibitors, such as AUY922, onalespib (AT13387), SNX-2112, SNX5422; murine double minute (mdm2) oncogene inhibitors, such as DS-3032b, RG7775, AMG-232, HDM201, and idasanutlin (RG7388); CD137 agonists, such as urelumab, utomilumab (PF-05082566), AGEN2373, ADG-106, BT-7480, QL1806; STING agonists, such as ADU-S100 (MIW-815), SB-11285, MK-1454, SR-8291, AdVCA0848, GSK-532, SYN-STING, MSA-1, SR-8291, GSK3745417; FGFR inhibitors, such as FGF-401, INCB-054828, BAY-1163877, AZD4547, JNJ-42756493, LY2874455, Debio-1347; fatty acid synthase (FASN) inhibitors, such as TVB-2640; CD44 binders, such as A6; protein phosphatease 2A (PP2A) inhibitors, such as LB-100; CYP17 inhibitors, such as seviteronel (VT-464), ASN-001, ODM-204, CFG920, abiraterone acetate; RXR agonists, such as IRX4204; hedgehog/smoothened (hh/Smo) antagonists, such as taladegib, patidegib, vismodegib; complement C3 modulators, such as Imprime PGG; IL-15 agonists, such as ALT-803, NKTR-255, interleukin-15/Fc fusion protein, AM-0015, NIZ-985, and hetIL-15; EZH2 (enhancer of zeste homolog 2) inhibitors, such as tazemetostat, CPI-1205, GSK-2816126, PF-06821497; oncolytic viruses, such as pelareorep, CG-0070, MV-NIS therapy, HSV-1716, DS-1647, VCN-01, ONCOS-102, TBI-1401, tasadenoturev (DNX-2401), vocimagene amiretrorepvec, RP-1, CVA21, Celyvir, LOAd-703, OBP-301, IMLYGIC®; DOT1L (histone methyltransferase) inhibitors, such as pinometostat (EPZ-5676); toxins such as Cholera toxin, ricin, Pseudomonas exotoxin, Bordetella pertussis adenylate cyclase toxin, diphtheria toxin, and caspase activators; DNA plasmids, such as BC-819; PLK inhibitors of PLK 1, 2, and 3, such as volasertib (PLK1); WEE1 inhibitors, such as AZD-1775 (adavosertib); Rho kinase (ROCK) inhibitors, such as AT13148, KD025; Inhibition of Apoptosis Protein (IAP) inhibitors, such as ASTX660, debio-1143, birinapant, APG-1387, LCL-161; RNA polymerase inhibitors, such has lurbinectedin (PM-1183), CX-5461; Tubulin inhibitors, such as PM-184, BAL-101553 (lisavanbulin), and OXI-4503, fluorapacin (AC-0001), plinabulin, vinflunine; Toll-like receptor 4 (TLR-4) agonists, such as G100, GSK1795091, and PEPA-10; Elongation factor 1 alpha 2 inhibitors, such as plitidepsin; Elongation factor 2 inhibitors/Interleukin-2 ligands/NAD ADP ribosyltransferase stimulators, such as denileukin diftitox; CD95 inhibitors, such as APG-101, APO-010, asunercept; WT1 inhibitors, such as DSP-7888; splicing factor 3B subunitl (SF3B1) inhibitors, such as H3B-8800; retinoid Z receptor gamma (RORγ) agonists, such as LYC-55716; and microbiome modulators, such as SER-401, EDP-1503, MRx-0518.

In various embodiments, the agent that inhibits binding between CD47 and SIRPα (e.g., magrolimab); and the NAE1 inhibitor (e.g., pevonedistat), as described herein, is further combined with one or more additional therapeutic agents comprising an inhibitor or antagonist of: myeloid cell leukemia sequence 1 (MCL1) apoptosis regulator (NCBI Gene ID: 4170); mitogen-activated protein kinase 1 (MAP4K1) (also called Hematopoietic Progenitor Kinase 1 (HPK1), NCBI Gene ID: 11184); diacylglycerol kinase alpha (DGKA, DAGK, DAGK1 or DGK-alpha; NCBI Gene ID: 1606); 5′-nucleotidase ecto (NTSE or CD73; NCBI Gene ID: 4907); ectonucleoside triphosphate diphosphohydrolase 1 (ENTPD1 or CD39; NCBI Gene ID: 593); transforming growth factor beta 1 (TGFB1 or TGFβ; NCBI Gene ID: 7040); heme oxygenase 1 (HMOX1, HO-1 or H01; NCBI Gene ID: 3162); heme oxygenase 2 (HMOX2, HO-2 or H02; NCBI Gene ID: 3163); vascular endothelial growth factor A (VEGFA or VEGF; NCBI Gene ID: 7422); erb-b2 receptor tyrosine kinase 2 (ERBB2, HER2, HER2/neu or CD340; NCBI Gene ID: 2064), epidermal growth factor receptor (EGFR, ERBB, ERBB1 or HER1; NCBI Gene ID: 1956); ALK receptor tyrosine kinase (ALK, CD246; NCBI Gene ID: 238); poly(ADP-ribose) polymerase 1 (PARP1; NCBI Gene ID: 142); poly(ADP-ribose) polymerase 2 (PARP2; NCBI Gene ID: 10038); TCDD inducible poly(ADP-ribose) polymerase (TIPARP, PARP7; NCBI Gene ID: 25976); cyclin dependent kinase 4 (CDK4; NCBI Gene ID: 1019); cyclin dependent kinase 6 (CDK6; NCBI Gene ID: 1021); TNF receptor superfamily member 14 (TNFRSF14, HVEM, CD270; NCBI Gene ID: 8764); T cell immunoreceptor with Ig and ITIM domains (TIGIT; NCBI Gene ID: 201633); X-linked inhibitor of apoptosis (XIAP, BIRC4, IAP-3; NCBI Gene ID: 331); baculoviral IAP repeat containing 2 (BIRC2, cIAP1; NCBI Gene ID: 329); baculoviral IAP repeat containing 3 (BIRC3, cIAP2; NCBI Gene ID: 330); baculoviral IAP repeat containing 5 (BIRC5, surviving; NCBI Gene ID: 332); C-C motif chemokine receptor 2 (CCR2, CD192; NCBI Gene ID: 729230); C-C motif chemokine receptor 5 (CCR5, CD195; NCBI Gene ID: 1234); C-C motif chemokine receptor 8 (CCR8, CDw198; NCBI Gene ID: 1237); C-X-C motif chemokine receptor 2 (CXCR2, CD182; NCBI Gene ID: 3579); C-X-C motif chemokine receptor 3 (CXCR3, CD182, CD183; NCBI Gene ID: 2833); C-X-C motif chemokine receptor 4 (CXCR4, CD184; NCBI Gene ID: 7852); arginase (ARG1 (NCBI Gene ID: 383), ARG2 (NCBI Gene ID: 384)), carbonic anhydrase (CA1 (NCBI Gene ID: 759), CA2 (NCBI Gene ID: 760), CA3 (NCBI Gene ID: 761), CA4 (NCBI Gene ID: 762), CASA (NCBI Gene ID: 763), CASB (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CA11 (NCBI Gene ID: 770), CA12 (NCBI Gene ID: 771), CA13 (NCBI Gene ID: 377677), CA14 (NCBI Gene ID: 23632)), prostaglandin-endoperoxide synthase 1 (PTGS1, COX-1; NCBI Gene ID: 5742), prostaglandin-endoperoxide synthase 2 (PTGS2, COX-2; NCBI Gene ID: 5743), secreted phospholipase A2, prostaglandin E synthase (PTGES, PGES; Gene ID: 9536), arachidonate 5-lipoxygenase (ALOX5, 5-LOX; NCBI Gene ID: 240) and/or soluble epoxide hydrolase 2 (EPHX2, SEH; NCBI Gene ID: 2053); a secreted phospholipase A2 (e.g., PLA2G1B (NCBI Gene ID: 5319); PLA2G7 (NCBI Gene ID: 7941), PLA2G3 (NCBI Gene ID: 50487), PLA2G2A (NCBI Gene ID: 5320); PLA2G4A (NCBI Gene ID: 5321); PLA2G12A (NCBI Gene ID: 81579); PLA2G12B (NCBI Gene ID: 84647); PLA2G10 (NCBI Gene ID: 8399); PLA2G5 (NCBI Gene ID: 5322); PLA2G2D (NCBI Gene ID: 26279); PLA2G15 (NCBI Gene ID: 23659)); indoleamine 2,3-dioxygenase 1 (IDO1; NCBI Gene ID: 3620); indoleamine 2,3-dioxygenase 2 (IDO2; NCBI Gene ID: 169355); hypoxia inducible factor 1 subunit alpha (HIF1A; NCBI Gene ID: 3091); angiopoietin 1 (ANGPT1; NCBI Gene ID: 284); Endothelial TEK tyrosine kinase (TIE-2, TEK, CD202B; NCBI Gene ID: 7010); Janus kinase 1 (JAK1; NCBI Gene ID: 3716); catenin beta 1 (CTNNB1; NCBI Gene ID: 1499); histone deacetylase 9 (HDAC9; NCBI Gene ID: 9734), and/or 5′-3′ exoribonuclease 1 (XRN1; NCBI Gene ID: 54464).

In various embodiments, the agent that inhibits binding between CD47 and SIRPα (e.g., magrolimab); and the NAE1 inhibitor (e.g., pevonedistat), as described herein, is further combined with an agonist of fms related receptor tyrosine kinase 3 (FLT3); FLK2; STK1; CD135; FLK-2; NCBI Gene ID: 2322). Examples of FLT3 agonists include, but are not limited to, CDX-301 and GS-3583.

In various embodiments, the agent that inhibits binding between CD47 and SIRPα (e.g., magrolimab); and the NAE1 inhibitor (e.g., pevonedistat), as described herein, is further combined with an anti-CD19 agent or antibody. Examples of anti-CD19 agents or antibodies that can be co-administered include without limitation: blinatumomab, tafasitamab, XmAb5574 (Xencor), AFM-11, inebilizumab, loncastuximab, MEDI 551 (Cellective Therapeutics); and MDX-1342 (Medarex).

In various embodiments, the agent that inhibits binding between CD47 and SIRPα (e.g., magrolimab); and the NAE1 inhibitor (e.g., pevonedistat), as described herein, is further combined with an anti-CD20 agent or antibody. Examples of anti-CD20 agents or antibodies that can be co-administered include without limitation: IGN-002, PF-05280586; Rituximab (Rituxan/Biogen Idec), Ofatumumab (Arzerra/Genmab), Obinutuzumab (Gazyva/Roche Glycart Biotech), Alemtuzumab, Veltuzumab, Veltuzumab, Ocrelizumab (Ocrevus/Biogen Idec; Genentech), Ocaratuzumab and Ublituximab, and LFB-R603 (LFB Biotech.; rEVO Biologics).

In various embodiments, the agent that inhibits binding between CD47 and SIRPα (e.g., magrolimab); and the NAE1 inhibitor (e.g., pevonedistat), as described herein, is further combined with an anti-CD22 agent or antibody. Examples of anti-CD22 agents or antibodies that can be co-administered include without limitation: Epratuzumab, AMG-412, IMMU-103 (Immunomedics).

In various embodiments, the agent that inhibits binding between CD47 and SIRPα (e.g., magrolimab); and the NAE1 inhibitor (e.g., pevonedistat), as described herein, is further combined with an anti-CD30 agent or antibody. Examples of anti-CD30 agents or antibodies that can be co-administered include without limitation: Brentuximab vedotin (Seattle Genetics).

In various embodiments, the agent that inhibits binding between CD47 and SIRPα (e.g., magrolimab); and the NAE1 inhibitor (e.g., pevonedistat), as described herein, is further combined with an anti-CD33 agent or antibody. Examples of anti-CD33 agents or antibodies that can be co-administered include without limitation: gemtuzumab, lintuzumab, vadastuximab, CIK-CAR.CD33; CD33CART, AMG-330 (CD33/CD3), AMG-673 (CD33/CD3), and GEM-333 (CD3/CD33), and IMGN-779.

In various embodiments, the agent that inhibits binding between CD47 and SIRPα (e.g., magrolimab); and the NAE1 inhibitor (e.g., pevonedistat), as described herein, is further combined with an anti-CD37 agent or antibody. Examples of anti-CD37 agents or antibodies that can be co-administered include without limitation: BI836826 (Boehringer Ingelheim), Otlertuzumab, and TRU-016 (Trubion Pharmaceuticals).

In various embodiments, the agent that inhibits binding between CD47 and SIRPα (e.g., magrolimab); and the NAE1 inhibitor (e.g., pevonedistat), as described herein, is further combined with an anti-CD38 agent or antibody. Examples of anti-CD38 agents or antibodies that can be co-administered include without limitation: CD38, such as T-007, UCART-38; Darzalex (Genmab), Daratumumab, JNJ-54767414 (Darzalex/Genmab), Isatuximab, SAR650984 (ImmunoGen), MOR202, MOR03087 (MorphoSys), TAK-079; and anti-CD38-attenukine, such as TAK573.

In various embodiments, the agent that inhibits binding between CD47 and SIRPα (e.g., magrolimab); and the NAE1 inhibitor (e.g., pevonedistat), as described herein, is further combined with an anti-CD52 agent or antibody. Examples of anti-CD52 agents or antibodies that can be co-administered include without limitation: anti-CD52 antibodies, such as Alemtuzumab (Campath/University of Cambridge).

In various embodiments, the agent that inhibits binding between CD47 and SIRPα (e.g., magrolimab); and the NAE1 inhibitor (e.g., pevonedistat), as described herein, is further combined with an anti-CD98 (4F2, FRP-1) agent or antibody. Examples of anti-CD98 agents or antibodies that can be co-administered include without limitation: IGN523 (Igenica).

In various embodiments, the agent that inhibits binding between CD47 and SIRPα (e.g., magrolimab); and the NAE1 inhibitor (e.g., pevonedistat), as described herein, is further combined with an anti-CD157 (BST-1) agent or antibody. Examples of anti-CD157 agents or antibodies that can be co-administered include without limitation: OBT357, MEN1112 (Menarini; Oxford BioTherapeutics).

In various embodiments, the agent that inhibits binding between CD47 and SIRPα (e.g., magrolimab); and the NAE1 inhibitor (e.g., pevonedistat), as described herein, is further combined with an anti-DKK-1 agent or antibody. Examples of anti-DKK-1 agents or antibodies that can be co-administered include without limitation: BHQ880 (MorphoSys; Novartis), and DKN-01, LY-2812176 (Eli Lilly).

In various embodiments, the agent that inhibits binding between CD47 and SIRPα (e.g., magrolimab); and the NAE1 inhibitor (e.g., pevonedistat), as described herein, is further combined with an anti-GRP78 (BiP) agent or antibody. Examples of anti-GRP78 agents or antibodies that can be co-administered include without limitation: PAT-SM6 (OncoMab GmbH).

In various embodiments, the agent that inhibits binding between CD47 and SIRPα (e.g., magrolimab); and the NAE1 inhibitor (e.g., pevonedistat), as described herein, is further combined with an anti-NOTCH1 agent or antibody. Examples of anti-NOTCH1 agents or antibodies that can be co-administered include without limitation: Brontictuzumab, OMP-52M51 (OncoMed Pharmaceuticals).

In various embodiments, the agent that inhibits binding between CD47 and SIRPα (e.g., magrolimab); and the NAE1 inhibitor (e.g., pevonedistat), as described herein, is further combined with an anti-ROR1 agent or antibody. Examples of anti-ROR1 agents or antibodies that can be co-administered include without limitation: Mapatumumab, TRM1, and HGS-1012 (Cambridge Antibody Technology).

In various embodiments, the agent that inhibits binding between CD47 and SIRPα (e.g., magrolimab); and the NAE1 inhibitor (e.g., pevonedistat), as described herein, is further combined with an anti-SLAMF7 (CS1, CD319) agent or antibody. Examples of anti-SLAMF7 agents or antibodies that can be co-administered include without limitation: Elotuzumab, HuLuc63, BMS-901608 (Empliciti/PDL BioPharma), Mogamulizumab (KW-0761).

In various embodiments, the agent that inhibits binding between CD47 and SIRPα (e.g., magrolimab); and the NAE1 inhibitor (e.g., pevonedistat), as described herein, is further combined with an anti-TNFRSF10A (DR4; APO2; CD261; TRAILR1; TRAILR-1) agent or antibody. Examples of anti-TNFRSF10A agents or antibodies that can be co-administered include without limitation: Mapatumumab, TRM1, and HGS-1012 (Cambridge Antibody Technology).

In various embodiments, the agent that inhibits binding between CD47 and SIRPα (e.g., magrolimab); and the NAE1 inhibitor (e.g., pevonedistat), as described herein, is further combined with an anti-Transferrin Receptor (TFRC; CD71) agent or antibody. Examples of anti-Transferrin Receptor agents or antibodies that can be co-administered include without limitation: E2.3/A27.15 (University of Arizona).

In various embodiments, the agent that inhibits binding between CD47 and SIRPα (e.g., magrolimab); and the NAE1 inhibitor (e.g., pevonedistat), as described herein, is further combined with an anti-EPHA3 agent or antibody. Examples of anti-EPHA3 agents or antibodies that can be co-administered include without limitation: Ifabotuzumab, KB004 (Ludwig Institute for Cancer Research).

In various embodiments, the agent that inhibits binding between CD47 and SIRPα (e.g., magrolimab); and the NAE1 inhibitor (e.g., pevonedistat), as described herein, is further combined with an anti-CCR4 agent or antibody. Examples of anti-CCR4 agents or antibodies that can be co-administered include without limitation: Mogamulizumab, KW-0761 (Poteligeo/Kyowa Hakko Kirin Co.).

In various embodiments, the agent that inhibits binding between CD47 and SIRPα (e.g., magrolimab); and the NAE1 inhibitor (e.g., pevonedistat), as described herein, is further combined with an anti-CXCR4 agent or antibody. Examples of anti-CXCR4 agents or antibodies that can be co-administered include without limitation: Ulocuplumab, BMS-936564, MDX-1338 (Medarex), and PF-06747143 (Pfizer).

In various embodiments, the agent that inhibits binding between CD47 and SIRPα (e.g., magrolimab); and the NAE1 inhibitor (e.g., pevonedistat), as described herein, is further combined with an anti-BAFF agent or antibody. Examples of anti-BAFF agents or antibodies that can be co-administered include without limitation: Tabalumab, LY2127399 (Eli Lilly).

In various embodiments, the agent that inhibits binding between CD47 and SIRPα (e.g., magrolimab); and the NAE1 inhibitor (e.g., pevonedistat), as described herein, is further combined with an anti-BAFF Receptor (BAFF-R) agent or antibody. Examples of anti-BAFF-R agents or antibodies that can be co-administered include without limitation: VAY736 (MorphoSys; Novartis).

In various embodiments, the agent that inhibits binding between CD47 and SIRPα (e.g., magrolimab); and the NAE1 inhibitor (e.g., pevonedistat), as described herein, is further combined with an anti-RANKL agent or antibody. Examples of anti-RANKL agents or antibodies that can be co-administered include without limitation: Denosumab, AMG-162 (Prolia; Ranmark; Xgeva/Amgen).

In various embodiments, the agent that inhibits binding between CD47 and SIRPα (e.g., magrolimab); and the NAE1 inhibitor (e.g., pevonedistat), as described herein, is further combined with an anti-IL-6 agent or antibody. Examples of anti-IL-6 agents or antibodies that can be co-administered include without limitation: Siltuximab, CNTO-328 (Sylvant/Centocor).

In various embodiments, the agent that inhibits binding between CD47 and SIRPα (e.g., magrolimab); and the NAE1 inhibitor (e.g., pevonedistat), as described herein, is further combined with an anti-IL-6 Receptor (IL-6R) agent or antibody. Examples of anti-IL-6R agents or antibodies that can be co-administered include without limitation: Tocilizumab, R-1569 (Actemra/Chugai Pharmaceutical; Osaka University), or AS-101 (CB-06-02, IVX-Q-101).

In various embodiments, the agent that inhibits binding between CD47 and SIRPα (e.g., magrolimab); and the NAE1 inhibitor (e.g., pevonedistat), as described herein, is further combined with an anti-IL3RA (CD123) agent or antibody. Examples of anti-IL3RA (CD123) agents or antibodies that can be co-administered include without limitation: CSL360 (CSL), talacotuzumab, JNJ-56022473, CSL362 (CSL); vibecotamab (XmAb14045; Xencor); KHK2823 (Kyowa Hakko Kirin Co.); APV0436 (CD123/CD3); flotetuzumab (CD123/CD3); JNJ-63709178 (CD123/CD3); and XmAb-14045 (CD123/CD3) (Xencor).

In various embodiments, the agent that inhibits binding between CD47 and SIRPα (e.g., magrolimab); and the NAE1 inhibitor (e.g., pevonedistat), as described herein, is further combined with an anti-IL2RA (CD25) agent or antibody. Examples of anti-IL2RA agents or antibodies that can be co-administered include without limitation: Basiliximab, SDZ-CHI-621 (Simulect/Novartis), and Daclizumab.

In various embodiments, the agent that inhibits binding between CD47 and SIRPα (e.g., magrolimab); and the NAE1 inhibitor (e.g., pevonedistat), as described herein, is further combined with an anti-IGF-1R (CD221) agent or antibody. Examples of anti-IGF-1R agents or antibodies that can be co-administered include without limitation: Ganitumab, AMG-479 (Amgen); Ganitumab, AMG-479 (Amgen), Dalotuzumab, MK-0646 (Pierre Fabre), and AVE1642 (ImmunoGen).

In various embodiments, the agent that inhibits binding between CD47 and SIRPα (e.g., magrolimab); and the NAE1 inhibitor (e.g., pevonedistat), as described herein, is further combined with an anti-GM-CSF (CSF2) agent or antibody. Examples of anti-GM-CSF agents or antibodies that can be co-administered include without limitation: Lenzilumab (a.k.a., KB003; KaloBios Pharmaceuticals).

In various embodiments, the agent that inhibits binding between CD47 and SIRPα (e.g., magrolimab); and the NAE1 inhibitor (e.g., pevonedistat), as described herein, is further combined with an anti-HGF agent or antibody. Examples of anti-HGF agents or antibodies that can be co-administered include without limitation: Ficlatuzumab, AV-299 (AVEO Pharmaceuticals).

In various embodiments, the agent that inhibits binding between CD47 and SIRPα (e.g., magrolimab); and the NAE1 inhibitor (e.g., pevonedistat), as described herein, is further combined with an anti-CD44 agent or antibody. Examples of anti-CD44 agents or antibodies that can be co-administered include without limitation: RG7356, RO5429083 (Chugai Biopharmaceuticals; Roche).

In various embodiments, the agent that inhibits binding between CD47 and SIRPα (e.g., magrolimab); and the NAE1 inhibitor (e.g., pevonedistat), as described herein, is further combined with an anti-VLA-4 (CD49d) agent or antibody. Examples of anti-VLA-4 agents or antibodies that can be co-administered include without limitation: Natalizumab, BG-0002-E (Tysabri/Elan Corporation).

In various embodiments, the agent that inhibits binding between CD47 and SIRPα (e.g., magrolimab); and the NAE1 inhibitor (e.g., pevonedistat), as described herein, is further combined with an anti-ICAM-1 (CD54) agent or antibody. Examples of anti-ICAM-1 agents or antibodies that can be co-administered include without limitation: BI-505 (BioInvent International).

In various embodiments, the agent that inhibits binding between CD47 and SIRPα (e.g., magrolimab); and the NAE1 inhibitor (e.g., pevonedistat), as described herein, is further combined with an anti-VEGF-A agent or antibody. Examples of anti-VEGF-A agents or antibodies that can be co-administered include without limitation: Bevacizumab (Avastin/Genentech; Hackensack University Medical Center).

In various embodiments, the agent that inhibits binding between CD47 and SIRPα (e.g., magrolimab); and the NAE1 inhibitor (e.g., pevonedistat), as described herein, is further combined with an anti-Endosialin (CD248, TEM1) agent or antibody. Examples of antiEndosialin agents or antibodies that can be co-administered include without limitation: Ontecizumab, MORAB-004 (Ludwig Institute for Cancer Research; Morphotek).

In various embodiments, the agent that inhibits binding between CD47 and SIRPα (e.g., magrolimab); and the NAE1 inhibitor (e.g., pevonedistat), as described herein, is further combined with an anti-CD79 agent or antibody. Examples of anti-CD79 agents or antibodies that can be co-administered include without limitation: polatuzumab, DCDS4501A, RG7596 (Genentech).

In various embodiments, the agent that inhibits binding between CD47 and SIRPα (e.g., magrolimab); and the NAE1 inhibitor (e.g., pevonedistat), as described herein, is further combined with an anti-Isocitrate dehydrogenase (IDH) agent or antibody. Examples of anti-IDH agents or antibodies that can be co-administered include without limitation: IDH1 inhibitor ivosidenib (Tibsovo; Agios) and the IDH2 inhibitor enasidenib (Idhifa; Celgene/Agios).

In various embodiments, the agent that inhibits binding between CD47 and SIRPα (e.g., magrolimab); and the NAE1 inhibitor (e.g., pevonedistat), as described herein, is further combined with an antibody that targets tumor associated calcium signal transducer 2 (TACSTD2) (NCBI Gene ID: 4070; EGP-1, EGP1, GA733-1, GA7331, GP50, M1S1, TROP2), such as sacituzumab, e.g., sacituzumab govitecan-hziy (TRODELVY™).

In various embodiments, the agent that inhibits binding between CD47 and SIRPα (e.g., magrolimab); and the NAE1 inhibitor (e.g., pevonedistat), as described herein, is further combined with an anti-major histocompatibility complex, class I, G (HLA-G; NCBI Gene ID: 3135) antibody, such as TTX-080.

In various embodiments, the agent that inhibits binding between CD47 and SIRPα (e.g., magrolimab); and the NAE1 inhibitor (e.g., pevonedistat), as described herein, is further combined with an anti-leukocyte immunoglobulin like receptor B2 (LILRB2, a.k.a., CD85D, ILT4; NCBI Gene ID: 10288) antibody, such as JTX-8064 or MK-4830.

### TNF Receptor Superfamily (TNFRSF) Member Agonists or Activators

In various embodiments, the agent that inhibits binding between CD47 and SIRPα (e.g., magrolimab); and the NAE1 inhibitor (e.g., pevonedistat), as described herein, is further combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628, each of which is hereby incorporated by reference in its entirety.

Examples anti-TNF receptor superfamily member 10b (TNFRSF10B, DR5, TRAILR2) antibodies that can be co-administered include without limitation, such as DS-8273, CTB-006, INBRX-109, and GEN-1029.

Examples of anti-TNFRSF5 (CD40) antibodies that can be co-administered include without limitation selicrelumab (RO7009789), mitazalimab (a.k.a., vanalimab, ADC-1013, JNJ-64457107), RG7876, SEA-CD40, APX-005M and ABBV-428, ABBV-927, and JNJ-64457107.

Examples of anti-TNFRSF7 (CD27) that can be co-administered include without limitation varlilumab (CDX-1127).

Examples of anti-TNFRSF9 (4-1BB, CD137) antibodies that can be co-administered include without limitation urelumab, utomilumab (PF-05082566), AGEN2373, and ADG-106, BT-7480, and QL1806.

Examples of anti-TNFRSF17 (BCMA) that can be co-administered include without limitation GSK-2857916.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628, each of which is hereby incorporated by reference in its entirety.

Example anti-TRAILR1, anti-TRAILR2, anti-TRAILR3, anti-TRAILR4 antibodies that can be co-administered include without limitation ABBV-621.

Examples of Bi-specific antibodies targeting TNFRSF family members that can be co-administered include without limitation PRS-343 (CD-137/HER2), AFM26 (BCMA/CD16A), AFM-13 (CD16/CD30), REGN-1979 (CD20/CD3), AMG-420 (BCMA/CD3), INHIBRX-105 (4-1BB/PDL1), FAP-4-IBBL (4-1BB/FAP), XmAb-13676 (CD3/CD20), RG-7828 (CD20/CD3), CC-93269 (CD3/BCMA), REGN-5458 (CD3/BCMA), and IMM-0306 (CD47/CD20), and AMG-424 (CD38.CD3).

Examples of inhibitors of PVR related immunoglobulin domain containing (PVRIG, CD112R) that can be co-administered include without limitation: COM-701.

Examples of inhibitors of T cell immunoreceptor with Ig and ITIM domains (TIGIT; NCBI Gene ID: 201633) that can be co-administered include without limitation: BMS-986207, RG-6058, AGEN-1307, and COM-902, etigilimab, tiragolumab (a.k.a., MTIG-7192A; RG-6058; RO 7092284), AGEN1777, IBI-939, AB154, MG1131 and E05884448 (EOS-448).

Examples of inhibitors of hepatitis A virus cellular receptor 2 (HAVCR2, TIMD3, TIM-3) that can be co-administered include without limitation: cobolimab (TSR-022), LY-3321367, sabatolimab (MBG-453), INCAGN-2390, RO-7121661 (PD-1/TIM-3), LY-3415244 (TIM-3/PDL1), and RG7769 (PD-1/TIM-3).

Examples of inhibitors of lymphocyte activating 3 (LAG-3, CD223) that can be co-administered include without limitation: relatlimab (ONO-4482), LAG-525, MK-4280, REGN-3767, INCAGN2385, TSR-033, MGD-013 (PD-1/LAG-3), and FS-118 (LAG-3/PD-L1).

Examples of anti-killer cell immunoglobulin like receptor, three Ig domains and long cytoplasmic tail 1 (KIR3DL1; KIR; NCBI Gene ID: 3811) monoclonal antibodies, such as lirilumab (IPH-2102), and IPH-4102.

Examples of anti-NKG2a antibodies that can be co-administered include without limitation: monalizumab.

Examples of anti-V-set immunoregulatory receptor (VSIR, B7H5, VISTA) antibodies that can be co-administered include without limitation: HMBD-002, and CA-170 (PD-L1/VISTA).

Examples of anti-CD70 antibodies that can be co-administered include without limitation: AMG-172.

Examples of anti-ICOS antibodies that can be co-administered include without limitation: JTX-2011, GSK3359609.

Examples of ICOS agonists that can be co-administered include without limitation: ICOS-L.COMP (Gariepy, J. et al. 106th Annu Meet Am Assoc Immunologists (AAI) (May 9-13, San Diego) 2019, Abst 71.5).

### Immune Checkpoint Inhibitors

In various embodiments, the agent that inhibits binding between CD47 and SIRPα (e.g., magrolimab); and the NAE1 inhibitor (e.g., pevonedistat), as described herein, is further combined with one or more immune checkpoint inhibitors. In some embodiments, the one or more immune checkpoint inhibitors is a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, HBM-4003, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors/antibodies of PD-L1 (CD274) or PD-1 (PDCD1) that can be co-administered include without limitation zimberelimab, pembrolizumab (KEYTRUDA®, MK-3477), nivolumab (OPDIVO®, BMS-936558, MDX-1106), cemiplimab, pidilizumab, spartalizumab (PDR-001), atezolizumab (RG-7446; TECENTRIQ, MPDL3280A), durvalumab (MEDI-4736), avelumab (MSB0010718C), tislelizumab (BGB-A317), toripalimab (JS-001), genolimzumab (CBT-501), camrelizumab (SHR-1210), dostarlimab (TSR-042), sintilimab 308), tislelizumab (BGB-A317), cemiplimab (REGN-2810), lambrolizumab (CAS Reg. No. 1374853-91-4), AMG-404, AMP-224, MEDI0680 (AMP-514), BMS-936559, CK-301, PF-06801591, GEN-1046 (PD-L1/4-1BB), GLS-010 (WBP-3055), AK-103 (HX-008), AK-105, CS-1003, HLX-10, MGA-012, BI-754091, AGEN-2034, JNJ-63723283, LZM-009, BCD-100, LY-3300054, SHR-1201, Sym-021, ABBV-181, PD1-PIK, BAT-1306, CX-072, CBT-502, MSB-2311, JTX-4014, BGB-A333, SHR-1316, CS-1001 (WBP-3155, KN-035, HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), RO-7247669 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM-3/PDL1), RG7769 (PD-1/TIM-3) and INBRX-105 (4-1BB/PDL1), GNS-1480 (PD-L1/EGFR), SCH-900475, PF-06801591, AGEN-2034, AK-105, PD1-PIK, BAT-1306, BMS-936559, CK-301, MEDI-0680, PDR001+Tafinlar+Mekinist®, and those described, e.g., in Intl. Patent Publ. Nos. WO2018195321, WO2020014643, WO2019160882, and WO2018195321.

In various embodiments, an anti-CD47 agent as described herein, is combined with an inhibitor of MCL1 apoptosis regulator, BCL2 family member (MCL1, TM; EAT; MCL1L; MCL1S; Mcl-1; BCL2L3; MCL1-ES; bcl2-L-3; mcl1/EAT; NCBI Gene ID: 4170). Examples of MCL1 inhibitors include AMG-176, AMG-397, S-64315, and AZD-5991, 483-LM, A-1210477, UMI-77, JKY-5-037, and those described in WO2018183418, WO2016033486, and WO2017147410.

### Toll-Like Receptor (TLR) Agonists

In various embodiments, an anti-CD47 agent or an anti-SIRPα agent as described herein, is combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation DS-0509, GS-9620, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). An TLR7/TLR8 agonist that can be co-administered is NKTR-262. Example TLR8 agonists that can be co-administered include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Example TLR9 agonists that can be co-administered include without limitation AST-008, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, leftolimod (MGN-1703), CYT-003, CYT-003-QbG10 and PUL-042. Examples of TLR3 agonist include rintatolimod, poly-ICLC, RIBOXXON®, Apoxxim, RIBOXXIM®, IPH-33, MCT-465, MCT-475, and ND-1.1.

Examples of TLR8 inhibitors include, but are not limited to, E-6887, IMO-8400, IMO-9200 and VTX-763.

Examples of TLR8 agonists include, but are not limited to, MCT-465, motolimod, GS-9688, and VTX-1463.

Examples of TLR9 agonists include but are not limited to, AST-008, IMO-2055, IMO-2125, lefitolimod, litenimod, MGN-1601, and PUL-042.

Examples of TLR7/TLR8 agonists include without limitation NKTR-262, IMO-4200, MEDI-9197 (telratolimod), and resiquimod.

Examples of TLR agonists include without limitation: lefitolimod, tilsotolimod, rintatolimod, DSP-0509, AL-034, G-100, cobitolimod, AST-008, motolimod, GSK-1795091, GSK-2245035, VTX-1463, GS-9688, LHC-165, BDB-001, RG-7854, telratolimod.

In some embodiments, the therapeutic agent is a stimulator of interferon genes (STING) In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, 5,6-dimethylxanthenone-4-acetic acid (DMXAA), cyclic-GAMP (cGAMP), and cyclic-di-AMP.

### TCR Signaling Modulators

In various embodiments, the agent that inhibits binding between CD47 and SIRPα (e.g., magrolimab); and the NAE1 inhibitor (e.g., pevonedistat), as described herein, is further combined with one or more agonist or antagonist of T-Cell Receptor (TCR) signaling modulators. Activation of T cells through the TCR and is essential for thymocyte development and effector T cell function. TCR activation promotes signaling cascades that ultimately determine cell fate through regulating cytokine production, cell survival, proliferation, and differentiation. Examples of TCR signaling modulators include without limitation CD2 (cluster of differentiation 2, LFA-2, T11, LFA-3 receptor), CD3 (cluster of differentiation 3), CD4 (cluster of differentiation 4), CD8 (cluster of differentiation 8), CD28 (cluster of differentiation 28), CD45 (PTPRC, B220, GP180), LAT (Linker for activation of T cells, LAT1), Lck, LFA-1 (ITGB2, CD18, LAD, LCAMB), Src, Zap-70, SLP-76, DGKalpha, CBL-b, CISH, HPK1. Examples of agonist of cluster of differentiation 3 (CD3) that can be co-administered include without limitation MGD015.

In various embodiments, the agent that inhibits binding between CD47 and SIRPα (e.g., magrolimab); and the NAE1 inhibitor (e.g., pevonedistat), as described herein, is further combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of cancer cells within the tumor microenvironment. Activation or stimulation of stimulatory immune check points can augment the effect of immune checkpoint inhibitors in cancer therapeutics. In various embodiments, the immune checkpoint proteins or receptors regulate T cell responses (e.g., reviewed in Xu, et al., J Exp Clin Cancer Res. (2018) 37:110). In various embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18(11):671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (PDL1, PD-L1); programmed cell death 1 (PDCD1, PD-1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM-3); galectin 9 (LGALS9); lymphocyte activating 3 (LAG-3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript 1L (RAET1L; ULBP6); lymphocyte activating 3 (CD223); killer cell immunoglobulin like receptor (KIR); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1).

In various embodiments, the agent that inhibits binding between CD47 and SIRPα (e.g., magrolimab); and the NAE1 inhibitor (e.g., pevonedistat), as described herein, is further combined with one or more blockers or inhibitors of one or more T-cell inhibitory immune checkpoint proteins or receptors. Illustrative T-cell inhibitory immune checkpoint proteins or receptors include without limitation CD274 (PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG-3, CD223); hepatitis A virus cellular receptor 2 (HAVCR2, TIMD3, TIM-3); galectin 9 (LGALS9); killer cell immunoglobulin like receptor (KIR); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1).

In various embodiments, the agent that inhibits binding between CD47 and SIRPα (e.g., magrolimab); and the NAE1 inhibitor (e.g., pevonedistat), as described herein, is further combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNF SF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu, et al., J Exp Clin Cancer Res. (2018) 37:110.

In various embodiments, the agent that inhibits binding between CD47 and SIRPα (e.g., magrolimab); and the NAE1 inhibitor (e.g., pevonedistat), as described herein, is further combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94).

In various embodiments, the agent that inhibits binding between CD47 and SIRPα (e.g., magrolimab); and the NAE1 inhibitor (e.g., pevonedistat), as described herein, is further combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis, et al., Semin Immunol. (2017) 31:64-75; Fang, et al., Semin Immunol. (2017) 31:37-54; and Chiossone, et al., Nat Rev Immunol. (2018) 18(11):671-688.

### Adenosine Generation and Signaling

In various embodiments, the agent that inhibits binding between CD47 and SIRPα (e.g., magrolimab); and the NAE1 inhibitor (e.g., pevonedistat), as described herein, is further combined with an agonist or antagonist of A1R, A2AR, A2BR, A3R, CD73, CD39, CD26; e.g., Adenosine A3 receptor (A3R) agonists, such as namodenoson (CF102); A2aR/A2bR antagonists, such as AB928; anti-CD73 antibodies, such as MEDI-9447 (oleclumab), CPX-006, IPH-53, BMS-986179, NZV-930, CPI-006; CD73 inhibitors, such as AB-680, PSB-12379, PSB-12441, PSB-12425, CB-708, and those described in Int Patent Publication No. WO19173692; CD39/CD73 inhibitors, such as PBF-1662; anti-CD39 antibodies, such as TTX-030; adenosine A2A receptor antagonists, such as CPI-444, AZD-4635, preladenant, PBF-509; and adenosine deaminase inhibitors, such as pentostatin, cladribine.

### Bi-Specific T-Cell Engagers

In various embodiments, the agent that inhibits binding between CD47 and SIRPα (e.g., magrolimab); and the NAE1 inhibitor (e.g., pevonedistat), as described herein, is further combined with a bi-specific T-cell engager (e.g., not having an Fc) or an anti-CD3 bi-specific antibody (e.g., having an Fc). Illustrative anti-CD3 bi-specific antibodies or BiTEs that can be co-administered include AMG-160 (PSMA/CD3), AMG-212 (PSMA/CD3), AMG-330 (CD33/CD3), AMG-420 (BCMA/CD3), AMG-427 (FLT3/CD3), AMG-562 (CD19/CD3), AMG-596 (EGFRvIII/CD3), AMG-701 (BCMA/CD3), AMG-757 (DLL3/CD3), JNJ-64052781 (CD19/CD3), AMG-211 (CEA/CD3), BLINCYTO® (CD19/CD3), RG7802 (CEA/CD3), ERY-974 (CD3/GPC3), huGD2-BsAb (CD3/GD2), PF-06671008 (Cadherins/CD3), APV0436 (CD123/CD3), ERY974, flotetuzumab (CD123/CD3), GEM333 (CD3/CD33), GEMoab (CD3/PSCA), REGN-1979 (CD20/CD3), REGN-5678 (PSMA/CD28), MCLA-117 (CD3/CLEC12A), JNJ-0819, JNJ-7564 (CD3/heme), JNJ-63709178 (CD123/CD3), MGD-007 (CD3/gpA33), MGD-009 (CD3/B7H3), IMCgp100 (CD3/gp100), XmAb-14045 (CD123/CD3), XmAb-13676 (CD3/CD20), XmAb-18087 (SSTR2/CD3), catumaxomab (CD3/EpCAM), REGN-4018 (MUC16/CD3), RG6026, RG6076, RG6194, RG-7828 (CD20/CD3), CC-93269 (CD3/BCMA), REGN-5458 (CD3/BCMA), GRB-1302 (CD3/Erbb2), GRB-1342 (CD38/CD3), PF-06863135 (BCMA/CD3), SAR440234 (CD3/CDw123). As appropriate, the anti-CD3 binding bi-specific molecules may or may not have an Fc. Illustrative bi-specific T-cell engagers that can be co-administered target CD3 and a tumor-associated antigen as described herein, including, e.g., CD19 (e.g., blinatumomab); CD33 (e.g., AMG330); CEA (e.g., MEDI-565); receptor tyrosine kinase-like orphan receptor 1 (ROR1) (Gohil, et al., Oncoimmunology. (2017) May 17; 6 (7):e1326437); PD-L1 (Horn, et al., Oncotarget. 2017 Aug. 3; 8(35):57964-57980); and EGFRvIII (Yang, et al., Cancer Lett. 2017 Sep. 10; 403:224-230).

### Bi- and Tri-Specific Natural Killer (NK)-Cell Engagers

In various embodiments, the agent that inhibits binding between CD47 and SIRPα (e.g., magrolimab); and the NAE1 inhibitor (e.g., pevonedistat), as described herein, is further combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). Illustrative anti-CD16 bi-specific antibodies, BiKEs or TriKEs that can be co-administered include AFM26 (BCMA/CD16A) and AFM-13 (CD16/CD30). As appropriate, the anti-CD16 binding bi-specific molecules may or may not have an Fc. Illustrative bi-specific NK-cell engagers that can be co-administered target CD16 and one or more tumor-associated antigens as described herein, including, e.g., CD19, CD20, CD22, CD30, CD33, CD123, EGFR, EpCAM, ganglioside GD2, HER2/neu, HLA Class II and FOLR1. BiKEs and TriKEs are described, e.g., in Felices, et al., Methods Mol Biol. (2016) 1441:333-346; Fang, et al., Semin Immunol. (2017) 31:37-54.

### Hematopoietic Progenitor Kinase 1 (HPK1) Inhibitors

In various embodiments, the agent that inhibits binding between CD47 and SIRPα (e.g., magrolimab); and the NAE1 inhibitor (e.g., pevonedistat), as described herein, is further combined with an inhibitor of mitogen-activated protein kinase kinase kinase kinase 1 (MAP4K1, HPK1; NCBI Gene ID: 11184). Examples of Hematopoietic Progenitor Kinase 1 (HPK1) inhibitors include without limitation, those described in WO-2018183956, WO-2018183964, WO-2018167147, WO-2018183964, WO-2016205942, WO-2018049214, WO-2018049200, WO-2018049191, WO-2018102366, WO-2018049152, WO2020092528, WO2020092621 and WO-2016090300.

### Apoptosis Signal-Regulating Kinase (ASK) Inhibitors

In various embodiments, the agent that inhibits binding between CD47 and SIRPα (e.g., magrolimab); and the NAE1 inhibitor (e.g., pevonedistat), as described herein, is further combined with an inhibitor of an ASK inhibitor, e.g., mitogen-activated protein kinase kinase kinase 5 (MAP3K5; ASK1, MAPKKK5, MEKK5; NCBI Gene ID: 4217). Examples of ASK1 inhibitors include without limitation, those described in WO 2011/008709 (Gilead Sciences) and WO 2013/112741 (Gilead Sciences).

### Bruton Tyrosine Kinase (BTK) Inhibitors

In various embodiments, the agent that inhibits binding between CD47 and SIRPα (e.g., magrolimab); and the NAE1 inhibitor (e.g., pevonedistat), as described herein, is further combined with an inhibitor of Bruton tyrosine kinase (BTK, AGMX1, AT, ATK, BPK, IGHD3, IMD1, PSCTK1, XLA; NCBI Gene ID: 695). Examples of BTK inhibitors include without limitation, (S)-6-amino-9-(1-(but-2-ynoyl)pyrrolidin-3-yl)-7-(4-phenoxyphenyl)-7H-purin-8(9H)-one, acalabrutinib (ACP-196), BGB-3111, CB988, HM71224, ibrutinib (Imbruvica), M-2951 (evobrutinib), M7583, tirabrutinib (ONO-4059), PRN-1008, spebrutinib (CC-292), TAK-020, vecabrutinib, ARQ-531, SHR-1459, DTRMWXHS-12, TAS-5315, Calquence+AZD6738, Calquence+danvatirsen.

### Cyclin-Dependent Kinase (CDK) Inhibitors

In various embodiments, the agent that inhibits binding between CD47 and SIRPα (e.g., magrolimab); and the NAE1 inhibitor (e.g., pevonedistat), as described herein, is further combined with an inhibitor of cyclin dependent kinase 1 (CDK1, CDC2; CDC28A; P34CDC2; NCBI Gene ID: 983); cyclin dependent kinase 2 (CDK2, CDKN2; p33 (CDK2); NCBI Gene ID: 1017); cyclin dependent kinase 3 (CDK3; NCBI Gene ID: 1018); cyclin dependent kinase 4 (CDK4, CMM3; PSK-J3; NCBI Gene ID: 1019); cyclin dependent kinase 6 (CDK6, MCPH12; PLSTIRE; NCBI Gene ID: 1021); cyclin dependent kinase 7 (CDK7, CAK; CAK1; HCAK; M015; STK1; CDKN7; p39MO15; NCBI Gene ID: 1022); cyclin dependent kinase 9 (CDK9, TAK; C-2k; CTK1; CDC2L4; PITALRE; NCBI Gene ID: 1025). Inhibitors of CDK 1, 2, 3, 4, 6, 7 and/or 9, include without limitation abemaciclib, alvocidib (HMR-1275, flavopiridol), AT-7519, dinaciclib, ibrance, FLX-925, LEE001, palbociclib, ribociclib, rigosertib, selinexor, UCN-01, SY1365, CT-7001, SY-1365, G1T38, milciclib, trilaciclib, PF-06873600, AZD4573, and TG-02.

### Discoidin Domain Receptor (DDR) Inhibitors.

In various embodiments, the agent that inhibits binding between CD47 and SIRPα (e.g., magrolimab); and the NAE1 inhibitor (e.g., pevonedistat), as described herein, is further combined with an inhibitor of discoidin domain receptor tyrosine kinase 1 (DDR1, CAK, CD167, DDR, EDDR1, HGK2, MCK10, NEP, NTRK4, PTK3, PTK3A, RTK6, TRKE; NCBI Gene ID: 780); and/or discoidin domain receptor tyrosine kinase 2 (DDR2, MIG20a, NTRKR3, TKT, TYRO10, WRCN; NCBI Gene ID: 4921). Examples of DDR inhibitors include without limitation, dasatinib and those disclosed in WO2014/047624 (Gilead Sciences), US 2009-0142345 (Takeda Pharmaceutical), US 2011-0287011 (Oncomed Pharmaceuticals), WO 2013/027802 (Chugai Pharmaceutical), and WO2013/034933 (Imperial Innovations).

### Histone Deacetylase (HDAC) Inhibitors

In various embodiments, the agent that inhibits binding between CD47 and SIRPα (e.g., magrolimab); and the NAE1 inhibitor (e.g., pevonedistat), as described herein, is further combined with an inhibitor of a histone deacetylase, e.g., histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CUDC-907 (fimepinostat), entinostat, givinostat, mocetinostat, panobinostat, pracinostat, quisinostat (JNJ-26481585), resminostat, ricolinostat, SHP-141, valproic acid (VAL-001), vorinostat, tinostamustine, remetinostat, entinostat, romidepsin, tucidinostat.

### Indoleamine-Pyrrole-2,3-Dioxygenase (IDO1) Inhibitors

In various embodiments, the agent that inhibits binding between CD47 and SIRPα (e.g., magrolimab); and the NAE1 inhibitor (e.g., pevonedistat), as described herein, is further combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, and shIDO-ST, EOS-200271, KHK-2455, LY-3381916.

### Janus Kinase (JAK) Inhibitors

In various embodiments, the agent that inhibits binding between CD47 and SIRPα (e.g., magrolimab); and the NAE1 inhibitor (e.g., pevonedistat), as described herein, is further combined with an inhibitor of Janus kinase 1 (JAK1, JAK1A, JAK1B, JTK3; NCBI Gene ID: 3716); Janus kinase 2 (JAK2, JTK10, THCYT3; NCBI Gene ID: 3717); and/or Janus kinase 3 (JAK3, JAK-3, JAK3 HUMAN, JAKL, L-JAK, LJAK; NCBI Gene ID: 3718). Examples of JAK inhibitors include without limitation, AT9283, AZD1480, baricitinib, BMS-911543, fedratinib, filgotinib (GLPG0634), gandotinib (LY2784544), INCB039110 (itacitinib), lestaurtinib, momelotinib (CYT0387), NS-018, pacritinib (SB1518), peficitinib (ASP015K), ruxolitinib, tofacitinib (formerly tasocitinib), INCB052793, and XL019.

### Matrix Metalloprotease (MMP) Inhibitors

In various embodiments, the agent that inhibits binding between CD47 and SIRPα (e.g., magrolimab); and the NAE1 inhibitor (e.g., pevonedistat), as described herein, is further combined with an inhibitor of a matrix metallopeptidase (MMP), e.g., an inhibitor of MMP1 (NCBI Gene ID: 4312), MMP2 (NCBI Gene ID: 4313), MMP3 (NCBI Gene ID: 4314), MMP7 (NCBI Gene ID: 4316), MMP8 (NCBI Gene ID: 4317), MMP9 (NCBI Gene ID: 4318); MMP10 (NCBI Gene ID: 4319); MMP11 (NCBI Gene ID: 4320); MMP12 (NCBI Gene ID: 4321), MMP13 (NCBI Gene ID: 4322), MMP14 (NCBI Gene ID: 4323), MMP15 (NCBI Gene ID: 4324), MMP16 (NCBI Gene ID: 4325), MMP17 (NCBI Gene ID: 4326), MMP19 (NCBI Gene ID: 4327), MMP20 (NCBI Gene ID: 9313), MMP21 (NCBI Gene ID: 118856), MMP24 (NCBI Gene ID: 10893), MMP25 (NCBI Gene ID: 64386), MMP26 (NCBI Gene ID: 56547), MMP27 (NCBI Gene ID: 64066) and/or MMP28 (NCBI Gene ID: 79148). Examples of MMP9 inhibitors include without limitation, marimastat (BB-2516), cipemastat (Ro 32-3555), GS-5745 (andecaliximab) and those described in WO 2012/027721 (Gilead Biologics).

### RAS and RAS Pathway Inhibitors

In various embodiments, the agent that inhibits binding between CD47 and SIRPα (e.g., magrolimab); and the NAE1 inhibitor (e.g., pevonedistat), as described herein, is further combined with an inhibitor of KRAS proto-oncogene, GTPase (KRAS; a.k.a., NS; NS3; CFC2; RALD; K-Ras; KRAS1; KRAS2; RASK2; KI-RAS; C—K-RAS; K-RAS2A; K-RAS2B; K-RAS4A; K-RAS4B; c-Ki-ras2; NCBI Gene ID: 3845); NRAS proto-oncogene, GTPase (NRAS; a.k.a., NS6; CMNS; NCMS; ALPS4; N-ras; NRAS1; NCBI Gene ID: 4893); HRas proto-oncogene, GTPase (HRAS; a.k.a., CTLO; KRAS; HAMSV; HRAS1; KRAS2; RASH1; RASK2; Ki-Ras; p21ras; C—H-RAS; c-K-ras; H-RASIDX; c-Ki-ras; C-BAS/HAS; C-HA-RAS1; NCBI Gene ID: 3265). The Ras inhibitors can inhibit Ras at either the polynucleotide (e.g., transcriptional inhibitor) or polypeptide (e.g., GTPase enzyme inhibitor) level. In some embodiments, the inhibitors target one or more proteins in the Ras pathway, e.g., inhibit one or more of EGFR, Ras, Raf (A-Raf, B-Raf, C-Raf), MEK (MEK1, MEK2), ERK, PI3K, AKT and mTOR.

In various embodiments, the agent that inhibits binding between CD47 and SIRPα (e.g., magrolimab); and the NAE1 inhibitor (e.g., pevonedistat), as described herein, is further combined with an inhibitor of KRAS. Examples of KRAS inhibitors include AMG-510, COTI-219, MRTX-1257, ARS-3248, ARS-853, WDB-178, BI-3406, BI-1701963, ARS-1620 (G12C), SML-8-73-1 (G12C), Compound 3144 (G12D), Kobe0065/2602 (Ras GTP), RT11, MRTX-849 (G12C) and K-Ras (G12D)-selective inhibitory peptides, including KRpep-2 (Ac-RRCPLYISYDPVCRR-NH2) (SEQ ID NO: 256) and KRpep-2d (Ac-RRRRCPLYISYDPVCRRRR-NH2) (SEQ ID NO: 257).

In various embodiments, the agent that inhibits binding between CD47 and SIRPα (e.g., magrolimab); and the NAE1 inhibitor (e.g., pevonedistat), as described herein, is further combined with an inhibitor of KRAS mRNA. Illustrative KRAS mRNA inhibitors include anti-KRAS U1 adaptor, AZD-4785, siG12D-LODER™, and siG12D exosomes.

In various embodiments, the agent that inhibits binding between CD47 and SIRPα (e.g., magrolimab); and the NAE1 inhibitor (e.g., pevonedistat), as described herein, is further combined with an inhibitor of MEK. Illustrative MEK inhibitors that can be co-administered include binimetinib, cobimetinib, PD-0325901, pimasertib, RG-7304, selumetinib, trametinib, and selumetinib.

In various embodiments, the agent that inhibits binding between CD47 and SIRPα (e.g., magrolimab); and the NAE1 inhibitor (e.g., pevonedistat), as described herein, is further combined with an inhibitor of AKT. Illustrative AKT inhibitors that can be co-administered include RG7440, MK-2206, ipatasertib, afuresertib, AZD5363, and ARQ-092, capivasertib, triciribine, ABTL-0812 (PI3K/Akt/mTOR).

In various embodiments, the agent that inhibits binding between CD47 and SIRPα (e.g., magrolimab); and the NAE1 inhibitor (e.g., pevonedistat), as described herein, is further combined with an inhibitor of Raf. Illustrative Raf inhibitors that can be co-administered BGB-283 (Raf/EGFR), HM-95573, LXH-254, LY-3009120, RG7304, TAK-580, dabrafenib, vemurafenib, encorafenib (LGX818), PLX8394. RAF-265 (Raf/VEGFR), ASN-003 (Raf/PI3K).

In various embodiments, the agent that inhibits binding between CD47 and SIRPα (e.g., magrolimab); and the NAE1 inhibitor (e.g., pevonedistat), as described herein, is further combined with an inhibitor of ERK. Illustrative ERK inhibitors that can be co-administered include LTT-462, LY-3214996, MK-8353, ravoxertinib, GDC-0994, and ulixertinib.

In various embodiments, the agent that inhibits binding between CD47 and SIRPα (e.g., magrolimab); and the NAE1 inhibitor (e.g., pevonedistat), as described herein, is further combined with an inhibitor of PI3K. Illustrative PI3K inhibitors that can be co-administered include idelalisib (Zydelig®), alpelisib, buparlisib, pictilisib, eganelisib (IPI-549). Illustrative PI3K/mTOR inhibitors that can be co-administered include dactolisib, omipalisib, voxtalisib, gedatolisib, GSK2141795, RG6114.

In various embodiments, the agent that inhibits binding between CD47 and SIRPα (e.g., magrolimab); and the NAE1 inhibitor (e.g., pevonedistat), as described herein, is further combined with an inhibitor of mTOR. Illustrative mTOR inhibitors that can be co-administered include as sapanisertib, vistusertib (AZD2014), ME-344, sirolimus (oral nano-amorphous formulation, cancer), TYME-88 (mTOR/cytochrome P450 3A4).

In certain embodiments, Ras-driven cancers (e.g., NSCLC) having CDKN2A mutations can be inhibited by co-administration of the MEK inhibitor selumetinib and the CDK4/6 inhibitor palbociclib. See, e.g., Zhou, et al., Cancer Lett. 2017 Nov. 1; 408:130-137. Also, K-RAS and mutant N-RAS can be reduced by the irreversible ERBB1/2/4 inhibitor neratinib. See, e.g., Booth, et al., Cancer Biol Ther. 2018 Feb. 1; 19(2):132-137.

In various embodiments, the agent that inhibits binding between CD47 and SIRPα (e.g., magrolimab); and the NAE1 inhibitor (e.g., pevonedistat), as described herein, is further combined with an inhibitor of RAS. Examples of RAS inhibitors include NEO-100, and rigosertib.

In various embodiments, the agent that inhibits binding between CD47 and SIRPα (e.g., magrolimab); and the NAE1 inhibitor (e.g., pevonedistat), as described herein, is further combined with an antagonist of EGFR, such as AMG-595, necitumumab, ABBV-221, depatuxizumab mafodotin (ABT-414), tomuzotuximab, ABT-806, vectibix, modotuximab, RM-1929.

In various embodiments, the agent that inhibits binding between CD47 and SIRPα (e.g., magrolimab); and the NAE1 inhibitor (e.g., pevonedistat), as described herein, is further combined with an inhibitor of protein tyrosine phosphatase non-receptor type 11 (PTPN11; BPTP3, CFC, JMML, METCDS, NS1, PTP-1D, PTP2C, SH-PTP2, SH-PTP3, SHP2; NCBI Gene ID: 5781). Examples of SHP2 inhibitors include TNO155 (SHP-099), RMC-4550, JAB-3068, RMC-4630, SAR442720 and those described in WO2018172984 and WO2017211303.

In various embodiments, the agent that inhibits binding between CD47 and SIRPα (e.g., magrolimab); and the NAE1 inhibitor (e.g., pevonedistat), as described herein, is further combined with an inhibitor of mitogen-activated protein kinase 7 (MAP2K7, JNKK2, MAPKK7, MEK, MEK 7, MKK7, PRKMK7, SAPKK-4, SAPKK4; NCBI Gene ID: 5609). Examples of MEK inhibitors include antroquinonol, binimetinib, CK-127, cobimetinib (GDC-0973, XL-518), MT-144, selumetinib (AZD6244), sorafenib, trametinib (GSK1120212), uprosertib+trametinib, PD-0325901, pimasertib, LTT462, AS703988, CC-90003, refametinib, TAK-733, CI-1040, RG7421.

In various embodiments, the agent that inhibits binding between CD47 and SIRPα (e.g., magrolimab); and the NAE1 inhibitor (e.g., pevonedistat), as described herein, is further combined with an inhibitor of a phosphatidylinositol-4,5-bisphosphate 3-kinase catalytic subunit, e.g., phosphatidylinositol-4,5-bisphosphate 3-kinase catalytic subunit alpha (PIK3CA, CLAPO, CLOVE, CWS5, MCAP, MCM, MCMTC, PI3K, PI3K-alpha, p110-alpha; NCBI Gene ID: 5290); phosphatidylinositol-4,5-bisphosphate 3-kinase catalytic subunit beta (PIK3CB, P110BETA, PI3K, PI3KBETA, PIK3C1; NCBI Gene ID: 5291); phosphatidylinositol-4,5-bisphosphate 3-kinase catalytic subunit gamma (PIK3CG, PI3CG, PI3K, PI3Kgamma, PIK3, p110gamma, p120-PI3K; Gene ID: 5494); and/or phosphatidylinositol-4,5-bisphosphate 3-kinase catalytic subunit delta (PIK3CD, APDS, IMD14, P110DELTA, PI3K, p110D, NCBI Gene ID: 5293). In some embodiments, the PI3K inhibitor is a pan-PI3K inhibitor. Examples of PI3K inhibitors include without limitation, ACP-319, AEZA-129, AMG-319, AS252424, AZD8186, BAY 1082439, BEZ235, bimiralisib (PQR309), buparlisib (BKM120), BYL719 (alpelisib), carboxyamidotriazole orotate (CTO), CH5132799, CLR-457, CLR-1401, copanlisib (BAY 80-6946), DS-7423, dactolisib, duvelisib (IPI-145), fimepinostat (CUDC-907), gedatolisib (PF-05212384), GDC-0032, GDC-0084 (RG7666), GDC-0077, pictilisib (GDC-0941), GDC-0980, GSK2636771, GSK2269577, GSK2141795, idelalisib (Zydelig®), INCB040093, INCB50465, IPI-443, IPI-549, KAR4141, LY294002, LY3023414, NERLYNX® (neratinib), nemiralisib (GSK2269557), omipalisib (GSK2126458, GSK458), OXY111A, panulisib (P7170, AK151761), PA799, perifosine (KRX-0401), Pilaralisib (SAR245408; XL147), puquitinib mesylate (XC-302), SAR260301, seletalisib (UCB-5857), serabelisib (INK-1117, MLN-1117, TAK-117), SF1126, sonolisib (PX-866), RG6114, RG7604, rigosertib sodium (ON-01910 sodium), RP5090, tenalisib (RP6530), RV-1729, SRX3177, taselisib, TG100115, umbralisib (TGR-1202), TGX221, voxtalisib (SAR245409), VS-5584, WX-037, X-339, X-414, XL499, XL756, wortmannin, ZSTK474, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences).

### Spleen Tyrosine Kinase (SYK) Inhibitors

In various embodiments, the agent that inhibits binding between CD47 and SIRPα (e.g., magrolimab); and the NAE1 inhibitor (e.g., pevonedistat), as described herein, is further combined with an inhibitor of spleen associated tyrosine kinase (SYK, p72-Syk, Gene ID: 6850). Examples of SYK inhibitors include without limitation, 6-(1H-indazol-6-yl)-N-(4-morpholinophenyl)imidazo[1,2-a]pyrazin-8-amine, BAY-61-3606, cerdulatinib (PRT-062607), entospletinib, fostamatinib (R788), HMPL-523, NVP-QAB 205 AA, R112, R343, tamatinib (R406), and those described in U.S. Pat. No. 8,450,321 (Gilead Connecticut) and those described in U.S. 2015/0175616.

### Tyrosine-Kinase Inhibitors (TKIs)

In various embodiments, the agent that inhibits binding between CD47 and SIRPα (e.g., magrolimab); and the NAE1 inhibitor (e.g., pevonedistat), as described herein, is further combined with a tyrosine kinase inhibitor (TKI). TKIs may target epidermal growth factor receptors (EGFRs) and receptors for fibroblast growth factor (FGF), platelet-derived growth factor (PDGF), and vascular endothelial growth factor (VEGF). Examples of TKIs include without limitation, axitinib, afatinib, ARQ-087 (derazantinib), asp5878, AZD3759, AZD4547, bosutinib, brigatinib, cabozantinib, cediranib, crenolanib, dacomitinib, dasatinib, dovitinib, E-6201, erdafitinib, erlotinib, gefitinib, gilteritinib (ASP-2215), FP-1039, HM61713, icotinib, imatinib, KX2-391 (Src), lapatinib, lestaurtinib, lenvatinib, midostaurin, nintedanib, ODM-203, olmutinib, osimertinib (AZD-9291), pazopanib, ponatinib, poziotinib, quizartinib, radotinib, rociletinib, sulfatinib (HMPL-012), sunitinib, famitinib L-malate, (MAC-4), tivoanib, TH-4000, tivoanib, and MEDI-575 (anti-PDGFR antibody), TAK-659, Cabozantinib.

### Chemotherapeutic Agents (Standard of Care)

In various embodiments, the agent that inhibits binding between CD47 and SIRPα (e.g., magrolimab); and the NAE1 inhibitor (e.g., pevonedistat), as described herein, is further combined with a chemotherapeutic agent or anti-neoplastic agent.

As used herein, the term “chemotherapeutic agent” or “chemotherapeutic” (or “chemotherapy” in the case of treatment with a chemotherapeutic agent) is meant to encompass any non-proteinaceous (e.g., non-peptidic) chemical compound useful in the treatment of cancer. Examples of chemotherapeutic agents include but not limited to: alkylating agents such as thiotepa and cyclophosphamide (CYTOXAN®); alkyl sulfonates such as busulfan, improsulfan, and piposulfan; aziridines such as benzodepa, carboquone, meturedepa, and uredepa; ethylenimines and methylamelamines including altretamine, triethylenemelamine, triethylenephosphoramide, triethylenethiophosphoramide, and trimemylolomelamine; acetogenins, e.g., bullatacin and bullatacinone; a camptothecin, including synthetic analog topotecan; bryostatin, callystatin; CC-1065, including its adozelesin, carzelesin, and bizelesin synthetic analogs; cryptophycins, particularly cryptophycin 1 and cryptophycin 8; dolastatin; duocarmycin, including the synthetic analogs KW-2189 and CBI-TMI; eleutherobin; 5-azacytidine; pancratistatin; a sarcodictyin; spongistatin; nitrogen mustards such as chlorambucil, chlornaphazine, cyclophosphamide, glufosfamide, evofosfamide, bendamustine, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, foremustine, lomustine, nimustine, and ranimustine; antibiotics such as the enediyne antibiotics (e.g., calicheamicin, especially calicheamicin gammaII and calicheamicin phiI1), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogs such as demopterin, methotrexate, pteropterin, and trimetrexate; purine analogs such as cladribine, pentostatin, fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replinishers such as frolinic acid; radiotherapeutic agents such as Radium-223, 177-Lu-PSMA-617; trichothecenes, especially T-2 toxin, verracurin A, roridin A, and anguidine; taxoids such as paclitaxel (TAXOL®), nab-paclitaxel (ABRAXANE®), docetaxel (TAXOTERE®), cabazitaxel, BIND-014, tesetaxel; platinum analogs such as cisplatin and carboplatin, NC-6004 nanoplatin; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; trabectedin, triaziquone; 2,2′,2″-trichlorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine (GEMZAR®); 6-thioguanine; mercaptopurine; methotrexate; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; NUC-1031; FOLFOX (folinic acid, 5-fluorouracil, oxaliplatin); FOLFIRI (folinic acid, 5-fluorouracil, irinotecan); FOLFOXIRI (folinic acid, 5-fluorouracil, oxaliplatin, irinotecan), FOLFIRINOX (folinic acid, 5-fluorouracil, irinotecan, oxaliplatin), and pharmaceutically acceptable salts, acids, or derivatives of any of the above. Such agents can be conjugated onto an antibody or any targeting agent described herein to create an antibody-drug conjugate (ADC) or targeted drug conjugate.

Also included in the definition of “chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors. Examples of anti-estrogens and SERMs include, for example, tamoxifen (including NOLVADEX™), raloxifene, droloxifene, 4-hydroxytamoxifen, trioxifene, keoxifene, LY117018, onapristone, and toremifene (FARESTON®). Inhibitors of the enzyme aromatase regulate estrogen production in the adrenal glands. Examples include 4(5)-imidazoles, aminoglutethimide, megestrol acetate (MEGACE®), exemestane, formestane, fadrozole, vorozole (RIVISOR®), letrozole (FEMARA®), and anastrozole (ARIMIDEX®). Examples of anti-androgens include apalutamide, abiraterone, enzalutamide, flutamide, galeterone, nilutamide, bicalutamide, leuprolide, goserelin, ODM-201, APC-100, ODM-204. An example progesterone receptor antagonist includes onapristone.

### Anti-Angiogenic Agents

In various embodiments, the agent that inhibits binding between CD47 and SIRPα (e.g., magrolimab); and the NAE1 inhibitor (e.g., pevonedistat), as described herein, is further combined with an anti-angiogenic agent. Anti-angiogenic agents that can be co-administered include, but are not limited to, retinoid acid and derivatives thereof, 2-methoxyestradiol, ANGIOSTATIN®, ENDOSTATIN®, regorafenib, necuparanib, suramin, squalamine, tissue inhibitor of metalloproteinase-1, tissue inhibitor of metalloproteinase-2, plasminogen activator inhibitor-1, plasminogen activator inhibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs such as 1-azetidine-2-carboxylic acid (LACA), cishydroxyproline, d,I-3,4-dehydroproline, thiaproline, α,α′-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carboxyphenyl-4-chloroanthronilic acid disodium or “CCA”, thalidomide, angiostatic steroid, carboxy aminoimidazole, metalloproteinase inhibitors such as BB-94, inhibitors of S100A9 such as tasquinimod. Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2.

### Anti-Fibrotic Agents

In various embodiments, the agent that inhibits binding between CD47 and SIRPα (e.g., magrolimab); and the NAE1 inhibitor (e.g., pevonedistat), as described herein, is further combined with an anti-fibrotic agent. Anti-fibrotic agents that can be co-administered include, but are not limited to, the compounds such as beta-aminoproprionitrile (BAPN), as well as the compounds disclosed in U.S. Pat. No. 4,965,288 relating to inhibitors of lysyl oxidase and their use in the treatment of diseases and conditions associated with the abnormal deposition of collagen and U.S. Pat. No. 4,997,854 relating to compounds which inhibit LOX for the treatment of various pathological fibrotic states, which are herein incorporated by reference. Further exemplary inhibitors are described in U.S. Pat. No. 4,943,593 relating to compounds such as 2-isobutyl-3-fluoro-, chloro-, or bromo-allylamine, U.S. Pat. Nos. 5,021,456, 5,059,714, 5,120,764, 5,182,297, 5,252,608 relating to 2-(1-naphthyloxymemyl)-3-fluoroallylamine, and US 2004-0248871, which are herein incorporated by reference.

Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone.

Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, particularly D-penicillamine, and its analogs such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

### Anti-Inflammatory Agents

In various embodiments, the agent that inhibits binding between CD47 and SIRPα (e.g., magrolimab); and the NAE1 inhibitor (e.g., pevonedistat), as described herein, is further combined with an anti-inflammatory agent. Example anti-inflammatory agents include without limitation inhibitors of one or more of arginase (ARG1 (NCBI Gene ID: 383), ARG2 (NCBI Gene ID: 384)), carbonic anhydrase (CA1 (NCBI Gene ID: 759), CA2 (NCBI Gene ID: 760), CA3 (NCBI Gene ID: 761), CA4 (NCBI Gene ID: 762), CASA (NCBI Gene ID: 763), CASB (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CA11 (NCBI Gene ID: 770), CA12 (NCBI Gene ID: 771), CA13 (NCBI Gene ID: 377677), CA14 (NCBI Gene ID: 23632)), prostaglandin-endoperoxide synthase 1 (PTGS1, COX-1; NCBI Gene ID: 5742), prostaglandin-endoperoxide synthase 2 (PTGS2, COX-2; NCBI Gene ID: 5743), secreted phospholipase A2, prostaglandin E synthase (PTGES, PGES; Gene ID: 9536), arachidonate 5-lipoxygenase (ALOX5, 5-LOX; NCBI Gene ID: 240), soluble epoxide hydrolase 2 (EPHX2, SEH; NCBI Gene ID: 2053) and/or mitogen-activated protein kinase kinase kinase 8 (MAP3K8, TPL2; NCBI Gene ID: 1326). In some embodiments, the inhibitor is a dual inhibitor, e.g., a dual inhibitor of COX-2/COX-1, COX-2/SEH, COX-2/CA, COX-2/5-LOX.

Examples of inhibitors of prostaglandin-endoperoxide synthase 1 (PTGS1, COX-1; NCBI Gene ID: 5742) that can be co-administered include without limitation mofezolac, GLY-230, and TRK-700.

Examples of inhibitors of prostaglandin-endoperoxide synthase 2 (PTGS2, COX-2; NCBI Gene ID: 5743) that can be co-administered include without limitation diclofenac, meloxicam, parecoxib, etoricoxib, AP-101, celecoxib, AXS-06, diclofenac potassium, DRGT-46, AAT-076, meisuoshuli, lumiracoxib, meloxicam, valdecoxib, zaltoprofen, nimesulide, Anitrazafen, Apricoxib, Cimicoxib, Deracoxib, Flumizole, Firocoxib, Mavacoxib, NS-398, Pamicogrel, Parecoxib, Robenacoxib, Rofecoxib, Rutecarpine, Tilmacoxib, and Zaltoprofen. Examples of dual COX1/COX2 inhibitors that can be co-administered include without limitation, HP-5000, lornoxicam, ketorolac tromethamine, bromfenac sodium, ATB-346, HP-5000. Examples of dual COX-2/carbonic anhydrase (CA) inhibitors that can be co-administered include without limitation polmacoxib and imrecoxib.

Examples of inhibitors of secreted phospholipase A2, prostaglandin E synthase (PTGES, PGES; Gene ID: 9536) that can be co-administered include without limitation LY3023703, GRC 27864, and compounds described in WO2015158204, WO2013024898, WO2006063466, WO2007059610, WO2007124589, WO2010100249, WO2010034796, WO2010034797, WO2012022793, WO2012076673, WO2012076672, WO2010034798, WO2010034799, WO2012022792, WO2009103778, WO2011048004, WO2012087771, WO2012161965, WO2013118071, WO2013072825, WO2014167444, WO2009138376, WO2011023812, WO2012110860, WO2013153535, WO2009130242, WO2009146696, WO2013186692, WO2015059618, WO2016069376, WO2016069374, WO2009117985, WO2009064250, WO2009064251, WO2009082347, WO2009117987, and WO2008071173. Metformin has further been found to repress the COX2/PGE2/STAT3 axis, and can be co-administered. See, e.g., Tong, et al., Cancer Lett. (2017) 389:23-32; and Liu, et al., Oncotarget. (2016) 7(19):28235-46.

Examples of inhibitors of carbonic anhydrase (e.g., one or more of CA1 (NCBI Gene ID: 759), CA2 (NCBI Gene ID: 760), CA3 (NCBI Gene ID: 761), CA4 (NCBI Gene ID: 762), CASA (NCBI Gene ID: 763), CASB (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CA11 (NCBI Gene ID: 770), CA12 (NCBI Gene ID: 771), CA13 (NCBI Gene ID: 377677), CA14 (NCBI Gene ID: 23632)) that can be co-administered include without limitation acetazolamide, methazolamide, dorzolamide, zonisamide, brinzolamide and dichlorphenamide. A dual COX-2/CA1/CA2 inhibitor that can be co-administered includes CG100649.

Examples of inhibitors of arachidonate 5-lipoxygenase (ALOX5, 5-LOX; NCBI Gene ID: 240) that can be co-administered include without limitation meclofenamate sodium, zileuton.

Examples of inhibitors of soluble epoxide hydrolase 2 (EPHX2, SEH; NCBI Gene ID: 2053) that can be co-administered include without limitation compounds described in WO2015148954. Dual inhibitors of COX-2/SEH that can be co-administered include compounds described in WO2012082647. Dual inhibitors of SEH and fatty acid amide hydrolase (FAAH; NCBI Gene ID: 2166) that can be co-administered include compounds described in WO2017160861.

Examples of inhibitors of mitogen-activated protein kinase kinase kinase 8 (MAP3K8, tumor progression loci-2, TPL2; NCBI Gene ID: 1326) that can be co-administered include without limitation GS-4875, GS-5290, BHM-078 and those described, e.g., in WO2006124944, WO2006124692, WO2014064215, WO2018005435, Teli, et al., J Enzyme Inhib Med Chem. (2012) 27(4):558-70; Gangwall, et al., Curr Top Med Chem. (2013) 13(9):1015-35; Wu, et al., Bioorg Med Chem Lett. (2009) 19(13):3485-8; Kaila, et al., Bioorg Med Chem. (2007) 15(19):6425-42; and Hu, et al., Bioorg Med Chem Lett. (2011) 21(16):4758-61.

### Tumor Oxygenation Agents

In various embodiments, the agent that inhibits binding between CD47 and SIRPα (e.g., magrolimab); and the NAE1 inhibitor (e.g., pevonedistat), as described herein, is further combined with an agent that promotes or increases tumor oxygenation or reoxygenation, or prevents or reduces tumor hypoxia. Illustrative agents that can be co-administered include, e.g., Hypoxia inducible factor-1 alpha (HIF-1α) inhibitors, such as PT-2977, PT-2385; VEGF inhibitors, such as bevasizumab, IMC-3C5, GNR-011, tanibirumab, LYN-00101, ABT-165; and/or an oxygen carrier protein (e.g., a heme nitric oxide and/or oxygen binding protein (HNOX)), such as OMX-302 and HNOX proteins described in WO 2007/137767, WO 2007/139791, WO 2014/107171, and WO 2016/149562.

### Immunotherapeutic Agents

In various embodiments, the agent that inhibits binding between CD47 and SIRPα (e.g., magrolimab); and the NAE1 inhibitor (e.g., pevonedistat), as described herein, is further combined with an immunotherapeutic agent. Example immunotherapeutic agents that can be co-administered include without limitation abagovomab, ABP-980, adecatumumab, afutuzumab, alemtuzumab, altumomab, amatuximab, anatumomab, arcitumomab, bavituximab, bectumomab, bevacizumab biosimilar, bivatuzumab, blinatumomab, brentuximab, cantuzumab, catumaxomab, CC49, cetuximab, citatuzumab, cixutumumab, clivatuzumab, conatumumab, dacetuzumab, dalotuzumab, daratumumab, detumomab, dinutuximab, drozitumab, duligotumab, dusigitumab, ecromeximab, emibetuzumab, ensituximab, ertumaxomab, etaracizumab, farletuzumab, figitumumab, flanvotumab, futuximab, gemtuzumab, girentuximab, glembatumumab, ibritumomab, igovomab, imgatuzumab, indatuximab, inotuzumab, intetumumab, ipilimumab (YERVOY®, MDX-010, BMS-734016, and MDX-101), iratumumab, labetuzumab, lexatumumab, lintuzumab, lorvotuzumab, lucatumumab, matuzumab, milatuzumab, minretumomab, mitumomab, moxetumomab, moxetumomab pasudotox, naptumomab, narnatumab, necitumumab, nimotuzumab, nofetumomab, OBI-833, obinutuzumab, ocaratuzumab, ofatumumab, olaratumab, onartuzumab, oportuzumab, oregovomab, panitumumab, parsatuzumab, pasudotox, patritumab, pemtumomab, pertuzumab, pintumomab, pritumumab, racotumomab, radretumab, ramucirumab (Cyramza®), rilotumumab, rituximab, robatumumab, samalizumab, satumomab, sibrotuzumab, siltuximab, solitomab, simtuzumab, tacatuzumab, taplitumomab, tenatumomab, teprotumumab, tigatuzumab, tositumomab, trastuzumab, trastuzumab biosimilar, tucotuzumab, ubilituximab, veltuzumab, vorsetuzumab, votumumab, zalutumumab, and 3F8. Rituximab can be used for treating indolent B-cell cancers, including marginal-zone lymphoma, WM, CLL and small lymphocytic lymphoma. A combination of Rituximab and chemotherapy agents is especially effective.

The exemplified therapeutic antibodies may be further labeled or combined with a radioisotope particle such as indium-111, yttrium-90 (90Y-clivatuzumab), or iodine-131.

In some embodiments, the immunotherapeutic agent is an antibody-drug conjugate (ADC). Illustrative ADCs that can be co-administered include without limitation drug-conjugated antibodies, fragments thereof, or antibody mimetics targeting the proteins or antigens listed above and herein (e.g., in Table B). Example ADCs that can be co-administered include without limitation gemtuzumab, brentuximab, trastuzumab, inotuzumab, glembatumumab, anetumab, mirvetuximab, depatuxizumab, rovalpituzumab, vadastuximab, labetuzumab, sacituzumab, lifastuzumab, indusatumab, polatzumab, pinatuzumab, coltuximab, indatuximab, milatuzumab, rovalpituzumab, ABBV-011, ABBV-2029, ABBV-321, ABBV-647, MLN0264 (anti-GCC, guanylyl cyclase C), T-DM1 (trastuzumab emtansine, Kadcycla); SYD985 (anti-HER2, Duocarmycin), milatuzumab-doxorubicin (hCD74-DOX), DCDT2980S, belantamab mafodotin (GSK2857916), polatuzumab vedotin (RG-7596), SGN-CD70A, SGN-CD19A, inotuzumab ozogamicin (CMC-544), lorvotuzumab mertansine, SAR3419, isactuzumab govitecan, enfortumab vedotin (ASG-22ME), ASG-15ME, DS-8201 ((trastuzumab deruxtecan), 225Ac-lintuzumab, U3-1402, 177Lu-tetraxetan-tetuloma, tisotumab vedotin, anetumab ravtansine, CX-2009, SAR-566658, W-0101, ABBV-085, gemtuzumab ozogamicin, ABT-414, glembatumumab vedotin (CDX-011), labetuzumab govitecan (IMMU-130), sacituzumab govitecan (IMMU-132; TRODELVY™), lifastuzumab vedotin, (RG-7599), milatuzumab-doxorubicin (IMMU-110), indatuximab ravtansine (BT-062), pinatuzumab vedotin (RG-7593), SGN-LIV1A, SGN-CD33A, SAR566658, MLN2704, SAR408701, rovalpituzumab tesirine, ABBV-399, AGS-16C3F, ASG-22ME, AGS67E, AMG 172, AMG 595, AGS-15E, BAY1129980, BAY1187982, BAY94-934 (anetumab ravtansine), GSK2857916, Humax-TF-ADC (tisotumab vedotin), IMGN289, IMGN529, IMGN853 (mirvetuximab soravtansine), LOP628, PCA062, MDX-1203, MEDI-547, PF-06263507, PF-06647020, PF-06647263, PF-06664178, PF-06688992, PF-06804103, RG7450, RG7458, RG7598, SAR566658, SGN-CD33A, DS-1602 and DS-7300, DS-6157, DS-6000, TAK-164, MEDI2228, MEDI7247, AMG575. ADCs that can be co-administered are described, e.g., in Lambert, et al., Adv Ther (2017) 34:1015-1035 and in de Goeij, Current Opinion in Immunology (2016) 40:14-23.

Illustrative therapeutic agents (e.g., anticancer or antineoplastic agents) that can be conjugated to the drug-conjugated antibodies, fragments thereof, or antibody mimetics include without limitation monomethyl auristatin E (MMAE), monomethyl auristatin F (MMAF), a calicheamicin, ansamitocin, maytansine or an analog thereof (e.g., mertansine/emtansine (DM1), ravtansine/soravtansine (DM4)), an anthracyline (e.g., doxorubicin, daunorubicin, epirubicin, idarubicin), pyrrolobenzodiazepine (PBD) DNA cross-linking agent SC-DR002 (D6.5), duocarmycin, a microtubule inhibitors (MTI) (e.g., a taxane, a vinca alkaloid, an epothilone), a pyrrolobenzodiazepine (PBD) or dimer thereof, a duocarmycin (A, B1, B2, C1, C2, D, SA, CC-1065), and other anticancer or anti-neoplastic agents described herein.

### Cancer Gene Therapy and Cell Therapy

In various embodiments, the agent that inhibits binding between CD47 and SIRPα (e.g., magrolimab); and the NAE1 inhibitor (e.g., pevonedistat), as described herein, is further combined with a cancer gene therapy and cell therapy. Cancer gene therapies and cell therapies include the insertion of a normal gene into cancer cells to replace a mutated or altered gene; genetic modification to silence a mutated gene; genetic approaches to directly kill the cancer cells; including the infusion of immune cells designed to replace most of the patient's own immune system to enhance the immune response to cancer cells, or activate the patient's own immune system (T cells or Natural Killer cells) to kill cancer cells, or find and kill the cancer cells; genetic approaches to modify cellular activity to further alter endogenous immune responsiveness against cancer.

### Cellular Therapies

In various embodiments, the agent that inhibits binding between CD47 and SIRPα (e.g., magrolimab); and the NAE1 inhibitor (e.g., pevonedistat), as described herein, is further combined with one or more cellular therapies. Illustrative cellular therapies include without limitation co-administration of one or more of a population of immune cells. In some embodiments, the immune cells are natural killer (NK) cells, NK-T cells, T cells, gamma delta T cells, B-cells, cytokine-induced killer (CIK) cells, macrophage (MAC) cells, tumor infiltrating lymphocytes (TILs) a granulocyte, an innate lymphoid cell, a megakaryocyte, a monocyte, a macrophage, a platelet, a thymocyte, a myeloid cell, and/or dendritic cells (DCs). In some embodiments, the cellular therapy entails a T cell therapy, e.g., co-administering a population of alpha/beta TCR T cells, gamma/delta TCR T cells, regulatory T (Treg) cells and/or TRuC™ T cells. In some embodiments, the cellular therapy entails a NK cell therapy, e.g., co-administering NK-92 cells. As appropriate, a cellular therapy can entail the co-administration of cells that are autologous, syngeneic or allogeneic to the subject.

In some embodiments, the cellular therapy entails co-administering immune cells engineered to express chimeric antigen receptors (CARs) or T cell receptors (TCRs) TCRs. In particular embodiments, a population of immune cells is engineered to express a CAR, wherein the CAR comprises a tumor antigen-binding domain. In other embodiments, a population of immune cells is engineered to express T cell receptors (TCRs) engineered to target tumor derived peptides presented on the surface of tumor cells. In one embodiment, the immune cell engineered to express chimeric antigen receptors (CARs) or T cell receptors (TCRs) TCRs is a T cell. In another embodiment, the immune cell engineered to express chimeric antigen receptors (CARs) or T cell receptors (TCRs) TCRs is an NK cell.

With respect to the structure of a CAR, in some embodiments, the CAR comprises an antigen binding domain, a transmembrane domain, and an intracellular signaling domain. In some embodiments, the intracellular domain comprises a primary signaling domain, a costimulatory domain, or both of a primary signaling domain and a costimulatory domain. In some embodiments, the primary signaling domain comprises a functional signaling domain of one or more proteins selected from the group consisting of CD3 zeta, CD3 gamma, CD3 delta, CD3 epsilon, common FcR gamma (FCERIG), FcR beta (Fc Epsilon R1b), CD79a, CD79b, Fcgamma RIIa, DAP10, and DAP12 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that binds with CD83, LIGHT, LIGHT, LTBR, Ly9 (CD229), Ly108), lymphocyte function-associated antigen-1 (LFA-1; CD1-1a/CD18), MEW class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

In some embodiments, the costimulatory domain comprises a functional domain of one or more proteins selected from the group consisting of CD27, CD28, 4-1BB (CD137), OX40, CD30, CD40, PD-1, ICOS, CD2, CD7, LIGHT, NKG2C, lymphocyte function-associated antigen-1 (LFA-1), MYD88, B7-H3, a ligand that specifically binds with CD83, CDS, ICAM-1, GITR, BAFFR, HVEM (LIGHTR), SLAMF7, NKp80 (KLRFI), CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, ITGAE, CD103, ITGAL, CD1A (NCBI Gene ID: 909), CD1B (NCBI Gene ID: 910), CD1C (NCBI Gene ID: 911), CD1D (NCBI Gene ID: 912), CD1E (NCBI Gene ID: 913), ITGAM, ITGAX, ITGB1, CD29, ITGB2 (CD18, LFA-1), ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

In some embodiments, the transmembrane domain comprises a transmembrane domain derived from a protein selected from the group consisting of the alpha, beta or zeta chain of the T-cell receptor, CD28, CD3 epsilon, CD3 delta, CD3 gamma, CD45, CD4, CD5, CD7, CD8 alpha, CD8 beta, CD9, CD11a, CD11b, CD11c, CD11d, CD16, CD18, CD22, CD33, CD37, CD64, CD80, CD86, CD134, CD137, CD154, KIRDS2, OX40, CD2, CD27, ICOS (CD278), 4-1BB (CD137), GITR, CD40, BAFFR, HVEM (LIGHTR), SLAMF7, NKp80 (KLRF1), CD19, CD19a, IL2R beta, IL2R gamma, IL7R alpha, ITGA1, VLA1, CD49a, ITGA4, IA4, CD49D, ITGA6, VLA-6, CD49f, ITGAD, CD1A, CD1B, CD1C, CD1D, CD1E, ITGAE, CD103, ITGAL, ITGAM, ITGAX, ITGB1, ITGB2, ITGB7, CD29, ITGB2 (LFA-1, CD18), ITGB7, TNFR2, DNAM1 (CD226), SLAMF4 (CD244, 2B4), CD84, CD96 (TACTILE), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C activating NK cell receptors, an Immunoglobulin protein, BTLA, CD247, CD276 (B7-H3), CD30, CD84, CDS, cytokine receptor, Fc gamma receptor, GADS, ICAM-1, Ig alpha (CD79a), integrins, LAT, a ligand that binds with CD83, LIGHT, MHC class 1 molecule, PAG/Cbp, TNFSF14, a Toll ligand receptor, TRANCE/RANKL, or a fragment, truncation, or a combination thereof.

In some embodiments, the CAR comprises a hinge domain. A hinge domain may be derived from a protein selected from the group consisting of the CD2, CD3 delta, CD3 epsilon, CD3 gamma, CD4, CD7, CD8.alpha, CD8.beta, CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD33? (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, or Toll ligand receptor, IgG1, IgG2, IgG3, IgG4, IgA, IgD, IgE, IgM or fragment or combination thereof.

In some embodiments, the TCR or CAR antigen binding domain or the immunotherapeutic agent described herein (e.g., monospecific or multi-specific antibody or antigen-binding fragment thereof or antibody mimetic) binds a tumor-associated antigen (TAA). In some embodiments, the tumor-associated antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECLI); CD33; epidermal growth factor receptor variant III (EGFRv111); ganglioside G2 (GD2); ganglioside GD3 (αNeuSAc(2-8)αNeuSAc(2-3)βDGaip(1-4)bDGIcp(1-1)Cer); ganglioside GM3 (αNeuSAc(2-3)βDGalp(1-4)βDGlcp(1-1)Cer); GM-CSF receptor; TNF receptor superfamily member 17 (TNFRSF17, BCMA); B-lymphocyte cell adhesion molecule; Tn antigen ((Tn Ag) or (GaINAcu-Ser/Thr)); prostate-specific membrane antigen (PSMA); Receptor tyrosine kinase-like orphan receptor 1 (RORI); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); HLA class I antigen A-2 alpha; HLA antigen; Lewis(Y)antigen; CD24; Platelet-derived growth factor receptor beta (PDGFR-beta); Stage-specific embryonic antigen-4 (SSEA-4); CD20; delta like 3 (DLL3); Folate receptor alpha; Folate receptor beta, GDNF alpha 4 receptor, Receptor tyrosine-protein kinase, ERBB2 (Her2/neu); Mucin 1, cell surface associated (MUC1); APRIL receptor; ADP ribosyl cyclase-1; Ephb4 tyrosine kinase receptor, DCAMKL1 serine threonine kinase, Aspartate beta-hydroxylase, epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); ephrin type-A receptor 3 (EphA3), Fucosyl GM1; sialyl Lewis adhesion molecule (sLe); transglutaminase 5 (TGS5); high molecular weight-melanomaassociatedantigen (HMWMAA); o-acetyl-GD2 ganglioside (OAcGD2); Folate receptor beta; tumor endothelial marker 1 (TEM1/CD248); tumor endothelial marker 7-related (TEM7R); six transmembrane epithelial antigen of the prostate I (STEAP1); claudin 6 (CLDN6); thyroid stimulating hormone receptor (TSHR); G protein-coupled receptor class C group 5, member D (GPRCSD); IL-15 receptor (IL-15); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (ORS IE2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma associated antigen 1 (MAGE-A1); Melanoma associated antigen 3 (MAGE-A3); Melanoma associated antigen 4 (MAGE-A4); T cell receptor beta 2 chain C; ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MADCT-1); melanoma cancer testis antigen-2 (MAD-CT-2); Fos-related antigen 1; tumor protein p53, (p53); p53 mutant; prostein; survivin; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin-A1; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP IBI); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAXS); proacrosin binding protein sp32 (OY-TES I); lymphocyte-specific protein tyrosine kinase (LCK); A kinase anchor protein 4 (AKAP-4); Peptidoglycan recognition protein, synovial sarcoma, X breakpoint 2 (SSX2); Receptor for Advanced Glycation Endproducts (RAGE-I); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module containing mucin-like hormone receptor-like 2 (EMR2); lymphocyte antigen 75 (LY75); Glypican-2 (GPC2); Glypican-3 (GPC3); Fc receptor-like 5 (FCRL5); and immunoglobulin lambda-like polypeptide 1 (IGLL1). In some embodiments, the target is an epitope of the tumor associated antigen presented in an MHC.

In some embodiments, the tumor antigen is selected from CD150, 5T4, ActRIIA, B7, TNF receptor superfamily member 17 (TNFRSF17, BCMA), CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, HER1-HER2 in combination, HER2-HER3 in combination, HER V-K, HIV-1 envelope glycoprotein gp120, HIV-1 envelope glycoprotein gp41, HLA-DR, HLA class I antigen alpha G, HM1.24, K-Ras GTPase, HMW-MAA, Her2, Her2/neu, IGF-1R, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NYESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-I, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D 1), ephrinB2, epithelial tumor antigen, estrogen receptor, fetal acetylcholine e receptor, folate binding protein, gp100, hepatitis B surface antigen, Epstein-Barr nuclear antigen 1, Latent membrane protein 1, Secreted protein BARF1, P2X7 purinoceptor, Syndecan-1, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, P2-Microgiobuiin, Fc Receptor-like 5 (FcRL5).

Examples of cell therapies include without limitation: AMG-119, Algenpantucel-L, ALOFISEL®, Sipuleucel-T, (BPX-501) rivogenlecleucel U.S. Pat. No. 9,089,520, WO2016100236, AU-105, ACTR-087, activated allogeneic natural killer cells CNDO-109-AANK, MG-4101, AU-101, BPX-601, FATE-NK100, LFU-835 hematopoietic stem cells, Imilecleucel-T, baltaleucel-T, PNK-007, UCARTCS1, ET-1504, ET-1501, ET-1502, ET-190, CD19-ARTEMIS, ProHema, FT-1050-treated bone marrow stem cell therapy, CD4CARNK-92 cells, SNK-01, NEXI-001, CryoStim, AlloStim, lentiviral transduced huCART-meso cells, CART-22 cells, EGFRt/19-28z/4-1BBL CAR T cells, autologous 4H11-28z/fIL-12/EFGRt T cell, CCR5-SBC-728-HSPC, CAR4-1BBZ, CH-296, dnTGFbRII-NY-ESOc259T, Ad-RTS-IL-12, IMA-101, IMA-201, CARMA-0508, TT-18, CMD-501, CMD-503, CMD-504, CMD-502, CMD-601, CMD-602, CSG-005, LAAP T-cell therapy, PD-1 knockout T cell therapy (esophageal cancer/NSCLC), anti-MUC1 CAR T-cell therapy (esophageal cancer/NSCLC), anti-MUC1 CAR T-cell therapy+PD-1 knockout T cell therapy (esophageal cancer/NSCLC), anti-KRAS G12D mTCR PBL, anti-CD123 CAR T-cell therapy, anti-mutated neoantigen TCR T-cell therapy, tumor lysate/MUC1/survivin PepTivator-loaded dendritic cell vaccine, autologous dendritic cell vaccine (metastatic malignant melanoma, intradermal/intravenous), anti-LeY-scFv-CD28-zeta CAR T-cells, PRGN-3005, iC9-GD2-CAR-IL-15 T-cells, HSC-100, ATL-DC-101, MIDRIX4-LUNG, MIDRIXNEO, FCR-001, PLX stem cell therapy, MDR-101, GeniusVac-Me14, ilixadencel, allogeneic mesenchymal stem cell therapy, romyelocel L, CYNK-001, ProTrans, ECT-100, MSCTRAIL, dilanubicel, FT-516, ASTVAC-2, E-CEL UVEC, CK-0801, allogenic alpha/beta CD3+ T cell and CD19+ B cell depleted stem cells (hematologic diseases, TBX-1400, HLCN-061, umbilical cord derived Hu-PHEC cells (hematological malignancies/aplastic anemia), AP-011, apceth-201, apceth-301, SENTI-101, stem cell therapy (pancreatic cancer), ICOVIR15-cBiTE, CD33HSC/CD33 CAR-T, PLX-Immune, SUBCUVAX, CRISPR allogeneic gamma-delta T-cell based gene therapy (cancer), ex vivo CRISPR allogeneic healthy donor NK-cell based gene therapy (cancer), ex-vivo allogeneic induced pluripotent stem cell-derived NK-cell based gene therapy (solid tumor), and anti-CD20 CAR T-cell therapy (non-Hodgkin's lymphoma).

### Additional Agents for Targeting Tumors

Additional agents for targeting tumors include without limitation: Alpha-fetoprotein modulators, such as ET-1402, and AFP-TCR; Anthrax toxin receptor 1 modulator, such as anti-TEM8 CAR T-cell therapy; TNF receptor superfamily member 17 (TNFRSF17, BCMA), such as bb-2121 (ide-cel), bb-21217, JCARH125, UCART-BCMA, ET-140, MCM-998, LCAR-B38M, CART-BCMA, SEA-BCMA, BB212, ET-140, P-BCMA-101, AUTO-2 (APRIL-CAR), JNJ-68284528; Anti-CLL-1 antibodies, (see, for example, PCT/US2017/025573); Anti-PD-L1-CAR tank cell therapy, such as KD-045; Anti-PD-L1 t-haNK, such as PD-L1 t-haNK; anti-CD45 antibodies, such as 131I-BC8 (lomab-B); anti-HER3 antibodies, such as LJM716, GSK2849330; APRIL receptor modulator, such as anti-BCMA CAR T-cell therapy, Descartes-011; ADP ribosyl cyclase-1/APRIL receptor modulator, such as dual anti-BCMA/anti-CD38 CAR T-cell therapy; CART-ddBCMA; B7 homolog 6, such as CAR-NKp30 and CAR-B7H6; B-lymphocyte antigen CD19, such as TBI-1501, CTL-119 huCART-19 T cells, 1 iso-cel, JCAR-015 U.S. Pat. No. 7,446,190, JCAR-014, JCAR-017, (WO2016196388, WO2016033570, WO2015157386), axicabtagene ciloleucel (KTE-C19, Yescarta®), KTE-X19, U.S. Pat. Nos. 7,741,465, 6,319,494, UCART-19, EBV-CTL, T tisagenlecleucel-T (CTL019), WO2012079000, WO2017049166, CD19CAR-CD28-CD3zeta-EGFRt-expressing T cells, CD19/4-1BBL armored CAR T cell therapy, C-CAR-011, CIK-CAR.CD19, CD19CAR-28-zeta T cells, PCAR-019, MatchCART, DSCAR-01, IM19 CAR-T, TC-110; anti-CD19 CAR T-cell therapy (B-cell acute lymphoblastic leukemia, Universiti Kebangsaan Malaysia); anti-CD19 CAR T-cell therapy (acute lymphoblastic leukemia/Non-Hodgkin's lymphoma, University Hospital Heidelberg), anti-CD19 CAR T-cell therapy (silenced IL-6 expression, cancer, Shanghai Unicar-Therapy Bio-medicine Technology), MB-CART2019.1 (CD19/CD20), GC-197 (CD19/CD7), CLIC-1901, ET-019003, anti-CD19-STAR-T cells, AVA-001, BCMA-CD19 cCAR (CD19/APRIL), ICG-134, ICG-132 (CD19/CD20), CTA-101, WZTL-002, dual anti-CD19/anti-CD20 CAR T-cells (chronic lymphocytic leukemia/B-cell lymphomas), HY-001, ET-019002, YTB-323, GC-012 (CD19/APRIL), GC-022 (CD19/CD22), CD19CAR-CD28-CD3zeta-EGFRt-expressing Tn/mem; UCAR-011, ICTCAR-014, GC-007F, PTG-01, CC-97540; allogeneic anti-CD19 CART cells, such as GC-007G; APRIL receptor modulator; SLAM family member 7 modulator, BCMA-CS1 cCAR; autologous dendritic cell tumor antigen (ADCTA), such as ADCTA-SSI-G; B-lymphocyte antigen CD20, such as ACTR707 ATTCK-20, PBCAR-20A; allogenic T cells expressing CD20 CAR, such as LB-1905; B-lymphocyte antigen CD19/B-lymphocyte antigen 22, such as TC-310; B-lymphocyte antigen 22 cell adhesion, such as UCART-22, JCAR-018 WO2016090190; NY-ESO-1 modulators, such as GSK-3377794, TBI-1301, GSK3537142; Carbonic anhydrase, such as DC-Ad-GMCAIX; Caspase 9 suicide gene, such as CaspaCIDe DLI, BPX-501; CCR5, such as SB-728; CCR5 gene inhibitor/TAT gene/TRIM5 gene stimulator, such as lentivirus vector CCR5 shRNA/TRIM5alpha/TAR decoy-transduced autologous CD34-positive hematopoietic progenitor cells; CDw123, such as MB-102, IM-23, JEZ-567, UCART-123; CD4, such as ICG-122; CD5 modulators, such as CD5.28z CART cells; Anti-CD22, such as anti-CD22 CART; Anti-CD30, such as TT-11; Dual anti-CD33/anti-CLL1, such as LB-1910; CD40 ligand, such as BPX-201, MEDI5083; CD56, such as allogeneic CD56-positive CD3-negative natural killer cells (myeloid malignancies); CD19/CD7 modulator, such as GC-197; T-cell antigen CD7 modulator, such as anti-CD7 CAR T-cell therapy (CD7-positive hematological malignancies); CD123 modulator, such as UniCAR02-T-CD123; Anti-CD276, such as anti-CD276 CART; CEACAM protein 5 modulators, such as MG7-CART; Claudin 6, such as CSG-002; Claudin 18.2, such as LB-1904; Chlorotoxin, such as CLTX-CART; EBV targeted, such as CMD-003; MUC16EGFR, such as autologous 4H11-28z/fIL-12/EFGRt T cell; Endonuclease, such as PGN-514, PGN-201; Epstein-Barr virus specific T-lymphocytes, such as TT-10; Epstein-Barr nuclear antigen 1/Latent membrane protein 1/Secreted protein BARF1 modulator, such as TT-10X; Erbb2, such as CST-102, CIDeCAR; Ganglioside (GD2), such as 4SCAR-GD2; Gamma delta T cells, such as ICS-200; folate hydrolase 1 (FOLH1, Glutamate carboxypeptidase II, PSMA; NCBI Gene ID: 2346), such as CIK-CAR.PSMA, CART-PSMA-TGFβRDN, P-PSMA-101; Glypican-3 (GPC3), such as TT-16, GLYCAR; Hemoglobin, such as PGN-236; Hepatocyte growth factor receptor, such as anti-cMet RNA CAR T; HLA class I antigen A-2 alpha modulator, such as FH-MCVA2TCR; HLA class I antigen A-2 alpha/Melanoma associated antigen 4 modulator, such as ADP-A2M4CD8; HLA antigen modulator, such as FIT-001, NeoTCR-P1; Human papillomavirus E7 protein, such as KITE-439 (see, for example, PCT/US2015/033129); ICAM-1 modulator, such as AIC-100; Immunoglobulin gamma Fc receptor III, such as ACTR087; IL-12, such as DC-RTS-IL-12; IL-12 agonist/mucin 16, such as JCAR-020; IL-13 alpha 2, such as MB-101; IL-15 receptor agonist, such as PRGN-3006, ALT-803; interleukin-15/Fc fusion protein (e.g., XmAb24306); recombinant interleukin-15 (e.g., AM0015, NIZ-985); pegylated IL-15 (e.g., NKTR-255); IL-2, such as CST-101; Interferon alpha ligand, such as autologous tumor cell vaccine+systemic CpG-B+IFN-alpha (cancer); K-Ras GTPase, such as anti-KRAS G12V mTCR cell therapy; Neural cell adhesion molecule L1 L1CAM (CD171), such as JCAR-023; Latent membrane protein 1/Latent membrane protein 2, such as Ad5f35-LMPd1-2-transduced autologous dendritic cells; MART-1 melanoma antigen modulator, such as MART-1 F5 TCR engineered PBMC; Melanoma associated antigen 10, such as MAGE-A10C796T MAGE-A10 TCR; Melanoma associated antigen 3/Melanoma associated antigen 6 (MAGE A3/A6) such as KITE-718 (see, for example, PCT/US2013/059608); Mesothelin, such as CSG-MESO, TC-210; Mucin 1 modulator, such as ICTCAR-052, Tn MUC-1 CAR-T, ICTCAR-053; Anti-MICA/MICB, such as CYAD-02; NKG2D, such as NKR-2; Ntrkr1 tyrosine kinase receptor, such as JCAR-024; PRAMET cell receptor, such as BPX-701; Prostate stem cell antigen modulator, such as MB-105; Roundabout homolog 1 modulator, such as ATCG-427; Peptidoglycan recognition protein modulator, such as Tag-7 gene modified autologous tumor cell vaccine; PSMA, such as PSMA-CAR T-cell therapy (lentiviral vector, castrate-resistant prostate cancer); SLAM family member 7 modulator, such as IC9-Luc90-CD828Z; TGF beta receptor modulator, such as DNR.NPC T-cells; T-lymphocyte, such as TT-12; T-lymphocyte stimulator, such as ATL-001; TSH receptor modulator, such as ICTCAR-051; Tumor infiltrating lymphocytes, such as LN-144, LN-145; and/or Wilms tumor protein, such as JTCR-016, WT1-CTL, ASP-7517.

### MCL1 Apoptosis Regulator, BCL2 Family Member (MCL1) Inhibitors

In various embodiments, an anti-CD47 agent or an anti-SIRPα agent as described herein, is combined with an inhibitor of MCL1 apoptosis regulator, BCL2 family member (MCL1, TM; EAT; MCL1L; MCL1S; Mcl-1; BCL2L3; MCL1-ES; bcl2-L-3; mcl1/EAT; NCBI Gene ID: 4170). Examples of MCL1 inhibitors include AMG-176, AMG-397, S-64315, and AZD-5991, 483-LM, A-1210477, UMI-77, JKY-5-037, and those described in WO2018183418, WO2016033486, WO2019222112 and WO2017147410.

### Cytokine Inducible SH2 Containing Protein (CISH) Inhibitors

In various embodiments, the agent that inhibits binding between CD47 and SIRPα (e.g., magrolimab); and the NAE1 inhibitor (e.g., pevonedistat), as described herein, is further combined with an inhibitor of cytokine inducible SH2 containing protein (CISH; CIS; G18; SOCS; CIS-1; BACTS2; NCBI Gene ID: 1154). Examples of CISH inhibitors include those described in WO2017100861, WO2018075664 and WO2019213610.

### Gene Editors

In various embodiments, the agent that inhibits binding between CD47 and SIRPα (e.g., magrolimab); and the NAE1 inhibitor (e.g., pevonedistat), as described herein, is further combined with gene editor. Illustrative gene editing system that can be co-administered include without limitation a CRISPR/Cas9 system, a zinc finger nuclease system, a TALEN system, a homing endonucleases system (e.g., an ARCUS), and a homing meganuclease system.

Other Drugs with Unspecified Targets

In various embodiments, the agent that inhibits binding between CD47 and SIRPα (e.g., magrolimab); and the NAE1 inhibitor (e.g., pevonedistat), as described herein, is further combined with human immunoglobulin (10% liquid formulation), Cuvitru (human immunoglobulin (20% solution), levofolinate disodium, IMSA-101, BMS-986288, IMUNO BGC Moreau RJ, R-OKY-034F, GP-2250, AR-23, calcium levofolinate, porfimer sodium, RG6160, ABBV-155, CC-99282, polifeprosan 20 with carmustine, Veregen, gadoxetate disodium, gadobutrol, gadoterate meglumine, gadoteridol, 99mTc-sestamibi, pomalidomide, pacibanil, and/or valrubicin.

### Exemplified Combination Therapies

### Lymphoma or Leukemia Combination Therapy

Some chemotherapy agents are suitable for treating lymphoma or leukemia. These agents include aldesleukin, alvocidib, amifostine trihydrate, aminocamptothecin, antineoplaston A10, antineoplaston AS2-1, anti-thymocyte globulin, arsenic trioxide, Bcl-2 family protein inhibitor ABT-263, beta alethine, BMS-345541bortezomib (VELCADE®, PS-341), bryostatin 1, bulsulfan, campath-1H, carboplatin, carfilzomib (Kyprolis®), carmustine, caspofungin acetate, CC-5103, chlorambucil, CHOP (cyclophosphamide, doxorubicin, vincristine, and prednisone), cisplatin, cladribine, clofarabine, curcumin, CVP (cyclophosphamide, vincristine, and prednisone), cyclophosphamide, cyclosporine, cytarabine, denileukin diftitox, dexamethasone, docetaxel, dolastatin 10, doxorubicin, doxorubicin hydrochloride, DT-PACE (dexamethasone, thalidomide, cisplatin, doxorubicin, cyclophosphamide, and etoposide), enzastaurin, epoetin alfa, etoposide, everolimus (RAD001), FCM (fludarabine, cyclophosphamide, and mitoxantrone), FCR (fludarabine, cyclophosphamide, and rituximab), fenretinide, filgrastim, flavopiridol, fludarabine, FR (fludarabine and rituximab), geldanamycin (17 AAG), hyperCVAD (hyperfractionated cyclophosphamide, vincristine, doxorubicin, dexamethasone, methotrexate, and cytarabine), ICE (iphosphamide, carboplatin, and etoposide), ifosfamide, irinotecan hydrochloride, interferon alpha-2b, ixabepilone, lenalidomide (REVLIMID®, CC-5013), pomalidomide (POMALYST®/IMNOVID®)lymphokine-activated killer cells, MCP (mitoxantrone, chlorambucil, and prednisolone), melphalan, mesna, methotrexate, mitoxantrone hydrochloride, motexafin gadolinium, mycophenolate mofetil, nelarabine, obatoclax (GX15-070), oblimersen, octreotide acetate, omega-3 fatty acids, Omr-IgG-am (WNIG, Omrix), oxaliplatin, paclitaxel, palbociclib (PD0332991), pegfilgrastim, PEGylated liposomal doxorubicin hydrochloride, perifosin, prednisolone, prednisone, recombinant flt3 ligand, recombinant human thrombopoietin, recombinant interferon alfa, recombinant interleukin-11, recombinant interleukin-12, rituximab, R-CHOP (rituximab and CHOP), R-CVP (rituximab and CVP), R-FCM (rituximab and FCM), R-ICE (rituximab and ICE), and R MCP (rituximab and MCP), R-roscovitine (seliciclib, CYC202), sargramostim, sildenafil citrate, simvastatin, sirolimus, styryl sulphones, tacrolimus, tanespimycin, temsirolimus (CC1-779), thalidomide, therapeutic allogeneic lymphocytes, thiotepa, tipifarnib, vincristine, vincristine sulfate, vinorelbine ditartrate, SAHA (suberanilohydroxamic acid, or suberoyl, anilide, and hydroxamic acid), vemurafenib (Zelboraf®), venetoclax (ABT-199).

One modified approach is radioimmunotherapy, wherein a monoclonal antibody is combined with a radioisotope particle, such as indium-111, yttrium-90, and iodine-131. Examples of combination therapies include, but are not limited to, iodine-131 tositumomab (BEXXAR®), yttrium-90 ibritumomab tiuxetan (ZEVALIN®), and BEXXAR® with CHOP.

The abovementioned therapies can be supplemented or combined with stem cell transplantation or treatment. Therapeutic procedures include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, low-LET cobalt-60 gamma ray therapy, bleomycin, conventional surgery, radiation therapy, and nonmyeloablative allogeneic hematopoietic stem cell transplantation.

### Non-Hodgkin's Lymphomas Combination Therapy

Treatment of non-Hodgkin's lymphomas (NHL), especially those of B cell origin, includes using monoclonal antibodies, standard chemotherapy approaches (e.g., CHOP (cyclophosphamide, doxorubicin, vincristine, and prednisone), CVP (cyclophosphamide, vincristine, and prednisone), FCM (fludarabine, cyclophosphamide, and mitoxantrone), MCP (Mitoxantrone, Chlorambucil, Prednisolone), all optionally including rituximab (R) and the like), radioimmunotherapy, and combinations thereof, especially integration of an antibody therapy with chemotherapy.

Examples of unconjugated monoclonal antibodies for the treatment of NHL/B-cell cancers include rituximab, alemtuzumab, human or humanized anti-CD20 antibodies, lumiliximab, anti-TNF-related apoptosis-inducing ligand (anti-TRAIL), bevacizumab, galiximab, epratuzumab, SGN-40, and anti-CD74.

Examples of experimental antibody agents used in treatment of NHL/B-cell cancers include ofatumumab, ha20, PRO131921, alemtuzumab, galiximab, SGN-40, CHIR-12.12, epratuzumab, lumiliximab, apolizumab, milatuzumab, and bevacizumab.

Examples of standard regimens of chemotherapy for NHL/B-cell cancers include CHOP, FCM, CVP, MCP, R-CHOP (rituximab, cyclophosphamide, doxorubicin, vincristine, and prednisone), R-FCM, R-CVP, and R MCP.

Examples of radioimmunotherapy for NHL/B-cell cancers include yttrium-90 ibritumomab tiuxetan (ZEVALIN®) and iodine-131 tositumomab (BEXXAR®).

### Mantle Cell Lymphoma Combination Therapy

Therapeutic treatments for mantle cell lymphoma (MCL) include combination chemotherapies such as CHOP, hyperCVAD, and FCM. These regimens can also be supplemented with the monoclonal antibody rituximab to form combination therapies R-CHOP, hyperCVAD-R, and R-FCM. Any of the abovementioned therapies may be combined with stem cell transplantation or ICE in order to treat MCL.

An alternative approach to treating MCL is immunotherapy. One immunotherapy uses monoclonal antibodies like rituximab. Another uses cancer vaccines, such as GTOP-99, which are based on the genetic makeup of an individual patient's tumor.

A modified approach to treat MCL is radioimmunotherapy, wherein a monoclonal antibody is combined with a radioisotope particle, such as iodine-131 tositumomab (BEXXAR®) and yttrium-90 ibritumomab tiuxetan (ZEVALIN®). In another example, BEXXAR® is used in sequential treatment with CHOP.

Other approaches to treating MCL include autologous stem cell transplantation coupled with high-dose chemotherapy, administering proteasome inhibitors such as bortezomib (VELCADE® or PS-341), or administering antiangiogenesis agents such as thalidomide, especially in combination with rituximab.

Another treatment approach is administering drugs that lead to the degradation of Bcl-2 protein and increase cancer cell sensitivity to chemotherapy, such as oblimersen, in combination with other chemotherapeutic agents.

A further treatment approach includes administering mTOR inhibitors, which can lead to inhibition of cell growth and even cell death. Non-limiting examples are sirolimus, temsirolimus (TORISEL®, CCI-779), CC-115, CC-223, SF-1126, PQR-309 (bimiralisib), voxtalisib, GSK-2126458, and temsirolimus in combination with RITUXAN®, VELCADE®, or other chemotherapeutic agents.

Other recent therapies for MCL have been disclosed. Such examples include flavopiridol, palbociclib (PD0332991), R-roscovitine (selicicilib, CYC202), styryl sulphones, obatoclax (GX15-070), TRAIL, Anti-TRAIL death receptors DR4 and DR5 antibodies, temsirolimus (TORISEL®, CC1-779), everolimus (RAD001), BMS-345541, curcumin, SAHA, thalidomide, lenalidomide (REVLIMID®, CC-5013), and geldanamycin (17 AAG).

### Waldenström's Macroglobulinemia Combination Therapy

Therapeutic agents used to treat Waldenström's Macroglobulinemia (WM) include aldesleukin, alemtuzumab, alvocidib, amifostine trihydrate, aminocamptothecin, antineoplaston A10, antineoplaston AS2-1, anti-thymocyte globulin, arsenic trioxide, autologous human tumor-derived HSPPC-96, Bcl-2 family protein inhibitor ABT-263, beta alethine, bortezomib (VELCADE®), bryostatin 1, busulfan, campath-1H, carboplatin, carmustine, caspofungin acetate, CC-5103, cisplatin, clofarabine, cyclophosphamide, cyclosporine, cytarabine, denileukin diftitox, dexamethasone, docetaxel, dolastatin 10, doxorubicin hydrochloride, DT-PACE, enzastaurin, epoetin alfa, epratuzumab (hLL2-anti-CD22 humanized antibody), etoposide, everolimus, fenretinide, filgrastim, fludarabine, ibrutinib, ifosfamide, indium-111 monoclonal antibody MN-14, iodine-131 tositumomab, irinotecan hydrochloride, ixabepilone, lymphokine-activated killer cells, melphalan, mesna, methotrexate, mitoxantrone hydrochloride, monoclonal antibody CD19 (such as ti sagenlecleucel-T, CART-19, CTL-019), monoclonal antibody CD20, motexafin gadolinium, mycophenolate mofetil, nelarabine, oblimersen, octreotide acetate, omega-3 fatty acids, oxaliplatin, paclitaxel, pegfilgrastim, PEGylated liposomal doxorubicin hydrochloride, pentostatin, perifosine, prednisone, recombinant flt3 ligand, recombinant human thrombopoietin, recombinant interferon alfa, recombinant interleukin-11, recombinant interleukin-12, rituximab, sargramostim, sildenafil citrate (VIAGRA®), simvastatin, sirolimus, tacrolimus, tanespimycin, thalidomide, therapeutic allogeneic lymphocytes, thiotepa, tipifarnib, tositumomab, ulocuplumab, veltuzumab, vincristine sulfate, vinorelbine ditartrate, vorinostat, WT1 126-134 peptide vaccine, WT-1 analog peptide vaccine, yttrium-90 ibritumomab tiuxetan, yttrium-90 humanized epratuzumab, and any combination thereof.

Examples of therapeutic procedures used to treat WM include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme techniques, low-LET cobalt-60 gamma ray therapy, bleomycin, conventional surgery, radiation therapy, and nonmyeloablative allogeneic hematopoietic stem cell transplantation.

### Diffuse Large B-Cell Lymphoma Combination Therapy

Therapeutic agents used to treat diffuse large B-cell lymphoma (DLBCL) include cyclophosphamide, doxorubicin, vincristine, prednisone, anti-CD20 monoclonal antibodies, etoposide, bleomycin, many of the agents listed for WM, and any combination thereof, such as ICE and RICE.

### Chronic Lymphocytic Leukemia Combination Therapy

Examples of therapeutic agents used to treat chronic lymphocytic leukemia (CLL) include chlorambucil, cyclophosphamide, fludarabine, pentostatin, cladribine, doxorubicin, vincristine, prednisone, prednisolone, alemtuzumab, many of the agents listed for WM, and combination chemotherapy and chemoimmunotherapy, including the following common combination regimens: CVP, R-CVP, ICE, R-ICE, FCR, and FR.

### Myelofibrosis Combination Therapy

Myelofibrosis inhibiting agents include, but are not limited to, hedgehog inhibitors, histone deacetylase (HDAC) inhibitors, and tyrosine kinase inhibitors. Non-limiting examples of hedgehog inhibitors are saridegib and vismodegib. Examples of HDAC inhibitors include, but are not limited to, pracinostat and panobinostat. Non-limiting examples of tyrosine kinase inhibitors are lestaurtinib, bosutinib, imatinib, radotinib, and cabozantinib.

### Hyperproliferative Disorder Combination Therapy

Gemcitabine, nab-paclitaxel, and gemcitabine/nab-paclitaxel may be used with a JAK inhibitor and/or PI3K6 inhibitor to treat hyperproliferative disorders.

3. Dosing and Scheduling

The methods described herein include administration of a therapeutically effective dose of compositions, e.g., a therapeutically effective dose of an agent that inhibits binding between CD47 and SIRPα and a therapeutically effective dose of an NAE1 inhibitor.

Compositions are administered to a patient in an amount sufficient to substantially ablate targeted cells, as described above. An amount adequate to accomplish this is defined as a “therapeutically effective dose,” which may provide for an improvement in overall survival rates. The term “therapeutically effective amount” is an amount that is effective to ameliorate a symptom of a disease (e.g., a cancer as described herein). A therapeutically effective amount can be a “prophylactically effective amount” as prophylaxis can be considered therapy. Single or multiple administrations of the compositions may be administered depending on the dosage and frequency as needed and tolerated by the patient. The particular dose used for a treatment will depend upon the medical condition and history of the mammal, as well as other factors such as age, weight, gender, administration route, efficiency, etc.

In some embodiments, combined therapeutic amounts of an agent that inhibits binding between CD47 and SIRPα; and a NEDD8-activating enzyme E1 regulatory subunit (NAE1) inhibitor, as described herein, optionally, with one or more additional therapeutic agents, as described herein, can (i) reduce the number of diseased cells; (ii) reduce tumor size; (iii) inhibit, retard, slow to some extent, and preferably stop the diseased cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with cancer or myeloproliferative disease. In some embodiments, combined therapeutic amounts of an agent that inhibits binding between CD47 and SIRPα; and a NEDD8-activating enzyme E1 regulatory subunit (NAE1) inhibitor, as described herein, optionally, with one or more additional therapeutic agents, as described herein, can (i) reduce the number of cancer cells; (ii) reduce tumor size; (iii) inhibit, retard, slow to some extent, and preferably stop cancer cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with the cancer. In various embodiments, the amount is sufficient to ameliorate, palliate, lessen, and/or delay one or more of symptoms of cancer.

An “increased” or “enhanced” amount (e.g., with respect to cancer cell proliferation or expansion, antitumor response, cancer cell metastasis) refers to an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

A “decreased” or “reduced” or “lesser” amount (e.g., with respect to tumor size, cancer cell proliferation or growth) refers to a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein.

An “anti-tumor effect” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence or recurrence of a tumor, e.g., a relapse after remission.

Effective doses of the combined agents for the treatment of cancer vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g., companion animals such as dogs, cats, horses, etc., laboratory mammals such as non-human primates, rabbits, mice, rats, etc., and the like. Treatment dosages can be titrated to optimize safety and efficacy.

A therapeutically effective dose of an anti-CD47 antibody can depend on the specific agent used, but is usually about 10 mg/kg body weight or more (e.g., about 10 mg/kg or more, about 15 mg/kg or more, 20 mg/kg or more, about 25 mg/kg or more, about 30 mg/kg or more, about 35 mg/kg or more, about 40 mg/kg or more, about 45 mg/kg or more, about 50 mg/kg or more, or about 55 mg/kg or more, or about 60 mg/kg or more, or about 65 mg/kg or more, or about 70 mg/kg or more), or from about 10 mg/kg, from about 15 mg/kg to about 70 mg/kg (e.g., from about 10 mg/kg to about 67.5 mg/kg, or from about 10 mg/kg, from about 15 mg/kg to about 60 mg/kg).

In some embodiments, the therapeutically effective dose of the anti-CD47 antibody is 10, 15, 20, 25, 30, 35, 40, 45, 50, 55, 60, 65, or 67.5 mg/kg. In some embodiments, the therapeutically effective dose of the anti-CD47 antibody is 10 to 60 mg/kg. In some embodiments, the therapeutically effective dose of the anti-CD47 antibody is 10 to 67.5 mg/kg. In some embodiments, the anti-CD47 antibody is administered at a dose of at least 10-30, 20-30, 15-60, 30-60, 10, 15, 20, 30, 40, 45, 50, or 60 mg of antibody per kg of body weight.

A therapeutic dose of an anti-CD47 antibody can be a flat dose. For example, a flat dose can be given irrespective of a particular subject's weight. Alternatively, a flat dose can be given based on a particular subject's weight falling within a particular weight range, e.g., a first range of less than or equal to 100 kg; or a second range of greater than 100 kg. A flat dose can be, e.g., 1000-5000, 2000-4000, 2000-3500, 2400-3500, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000 mg, or an interim number of mg thereof.

Methods can include a step of administering a primer agent to subject, followed by a step of administering a therapeutically effective dose of an anti-CD47 to the subject. In some embodiments, the step of administering a therapeutically effective dose is performed after at least about 3 days (e.g., at least about 4 days, at least about 5 days, at least about 6 days, at least about 7 days, at least about 8 days, at least about 9 days, or at least about 10 days) after beginning the administration of a primer agent. This period of time is, for example, sufficient to provide for enhanced reticulocyte production by the individual. In some embodiments, the anti-CD47 agent is an isolated anti-CD47 antibody.

The administration of a therapeutically effective dose of an anti-CD47 can be achieved in a number of different ways. In some cases, two or more therapeutically effective doses are administered after a primer agent is administered. Suitable administration of a therapeutically effective dose can entail administration of a single dose, or can entail administration of doses daily, semi-weekly, weekly, once every two weeks, once a month, annually, etc. In some cases, a therapeutically effective dose is administered as two or more doses of escalating concentration (i.e., increasing doses), where (i) all of the doses are therapeutic doses, or where (ii) a sub-therapeutic dose (or two or more sub-therapeutic doses) is initially given and therapeutic doses are achieved by said escalation. As one non-limiting example to illustrate escalating concentration (i.e., increasing doses), a therapeutically effective dose can be administered weekly, beginning with a sub-therapeutic dose (e.g., a dose of less than 10 mg/kg, e.g., 5 mg/kg, 4 mg/kg, 3 mg/kg, 2 mg/kg or 1 mg/kg), and each subsequent dose can be increased by a particular increment (e.g., by 5 mg/kg, by 10 mg/kg, by 15 mg/kg), or by variable increments, until a therapeutic dose (e.g., 15 mg/kg, 30 mg/kg, 45 mg/kg, 60 mg/kg) is reached, at which point administration may cease or may continue with one or more additional therapeutic doses (e.g., continued therapeutic doses or escalated therapeutic doses, e.g., doses of 15 mg/kg, 30 mg/kg, 45 mg/kg, 60 mg/kg). As another non-limiting example to illustrate escalating concentration (i.e., increasing doses), a therapeutically effective dose can be administered weekly, beginning with one or more relatively lower therapeutic doses (e.g., a dose of 10 mg/kg, 15 mg/kg or 30 mg/kg), and each subsequent dose can be increased by a particular increment (e.g., by 10 mg/kg or 15 mg/kg), or by variable increments, until a relatively higher therapeutic dose (e.g., 30 mg/kg, 45 mg/kg, 60 mg/kg, 100 mg/kg, etc.) is reached, at which point administration may cease or may continue (e.g., one or more continued or escalated therapeutic doses, e.g., doses of 30 mg/kg, 45 mg/kg, 60 mg/kg, 100 mg/kg, etc.). In various embodiments, relatively lower therapeutic doses are administered more often (e.g., two or more doses of 15 mg/kg administered weekly (Q1W) or two or more doses of 30 mg/kg administered every two weeks (Q2W)), and relatively higher therapeutic doses are administered less often (e.g., two or more doses of 45 mg/kg administered every 3 weeks (Q3W) or two or more doses of 60 mg/kg administered monthly or every 4 weeks (Q4W)). In some embodiments, administration of a therapeutically effective dose can be a continuous infusion and the dose can altered (e.g., escalated) over time.

The dose needed to achieve and/or maintain a particular serum level of the administered composition is proportional to the amount of time between doses and inversely proportional to the number of doses administered. Thus, as the frequency of dosing increases, the needed dose decreases. The optimization of dosing strategies will be readily understood and practiced by one of ordinary skill in the art. An exemplary treatment regime entails administration once every two weeks or once a month or once every 3 to 6 months. Therapeutic entities described herein are usually administered on multiple occasions. Intervals between single dosages can be weekly, monthly or yearly. Intervals can also be irregular as indicated by measuring blood levels of the therapeutic entity in the patient. Alternatively, therapeutic entities described herein can be administered as a sustained release formulation, in which case less frequent administration is used. Dosage and frequency vary depending on the half-life of the polypeptide in the patient. In some embodiments, the interval between each single dose is a week. In some embodiments, the interval between each single dose is two weeks. In some embodiments, the interval between each single dose is three weeks. In some embodiments, the interval between each single dose is four weeks. In some embodiments, the interval between each single dose of anti-CD47 antibody is a week. In some embodiments, the interval between each single dose of anti-CD47 antibody is two weeks. In some embodiments, the interval between each single dose of anti-CD47 antibody is three weeks. In some embodiments, the interval between each single dose of anti-CD47 antibody is four weeks. In some embodiments, the interval between each single dose of magrolimab is a week. In some embodiments, the interval between each single dose of magrolimab is two weeks. In some embodiments, the interval between each single dose of magrolimab is three weeks. In some embodiments, the interval between each single dose of magrolimab is four weeks.

A “maintenance dose” is a dose intended to be a therapeutically effective dose. For example, in experiments to determine the therapeutically effective dose, multiple different maintenance doses may be administered to different subjects. As such, some of the maintenance doses may be therapeutically effective doses and others may be sub-therapeutic doses.

In prophylactic applications, a relatively low dosage may be administered at relatively infrequent intervals over a long period of time. Some patients continue to receive treatment for the rest of their lives. In other therapeutic applications, a relatively high dosage at relatively short intervals is sometimes used until progression of the disease is reduced or terminated, and preferably until the patient shows partial or complete amelioration of symptoms of disease. Thereafter, the patent can be administered a prophylactic regime.

An initial dose of a CD47 or SIRPα binding agent, including but not limited to a priming dose, may lead to anemia or hemagglutination for a period of time immediately following infusion. Without being bound by the theory, it is believed that the initial dose of a multivalent CD47 or SIRPα binding agent may cause cross-linking of RBC bound to the agent. In certain embodiments, a CD47 or SIRPα binding agent is infused to a patient in an initial dose, and optionally in subsequent doses, over a period of time and/or concentration that reduces the possibility of hematologic microenvironments where there is a high local concentration of RBC and the agent.

The term “priming dose” or as used herein refers to a dose of an anti-CD47 antibody that primes a subject for administration of a therapeutically effective dose of anti-CD47 antibody such that the therapeutically effective dose does not result in a severe loss of RBCs (reduced hematocrit or reduced hemoglobin). The specific appropriate priming dose of an anti-CD47 antibody can vary depending on the nature of the agent used and on numerous subject-specific factors (e.g., age, weight, etc.). Examples of suitable priming doses of an anti-CD47 antibody include from about 0.5 mg/kg to about 5 mg/kg, from about 0.5 mg/kg to about 4 mg/kg, from about 0.5 mg/kg to about 3 mg/kg, from about 1 mg/kg to about 5 mg/kg, from about 1 mg/kg to about 4 mg/kg, from about 1 mg/kg to about 3 mg/kg, about 1 mg/kg, about 2 mg/kg, about 3 mg/kg, about 4 mg/kg, about 5 mg/kg. In some embodiments, the priming dose is preferably 1 mg/kg.

In some embodiments of the methods described herein, the anti-CD47 antibody is administered to the subject as a priming dose ranging from about 0.5 mg to about 10 mg, e.g., from about 0.5 to about 5 mg/kg of antibody, optionally, 4 mg/kg, 3 mg/kg, 2 mg/kg, or 1 mg/kg of antibody. In some embodiments, the anti-CD47 antibody is administered to the subject as a therapeutic dose ranging from about 20 to about 67.5 mg/kg of antibody, optionally from 15 to 60 mg/kg of antibody, optionally from 30 to 60 mg/kg of antibody, optionally 15 mg/kg of antibody, 20 mg/kg of antibody, 30 mg/kg of antibody, 45 mg/kg of antibody, 60 mg/kg of antibody, or 67.5 mg/kg of antibody.

A priming dose of an anti-CD47 antibody can be a flat priming dose. For example, a flat priming dose can be given irrespective of a particular subject's weight. Alternatively, a flat priming dose can be given based on a particular subject's weight falling within a particular weight range, e.g., a first range of less than or equal to 100 kg; or a second range of greater than 100 kg. A flat priming dose can be, e.g., 10-200, 50-100, 80-800, 80-400, 80-200, 70-90, 75-85, 10, 20, 30, 40, 50, 60, 70, 80, 90, 100, 110, 120, 130, 140, 150, 160, 170, 180, 190, 200, 240, 300, 320, 400, 500, 600, 700 or 800 mg, or an interim number of mg thereof.

In some embodiments, an effective priming dose of magrolimab is provided, where the effective priming dose for a human is around about 1 mg/kg, e.g., from at least about 0.5 mg/kg up to not more than about 5 mg/kg; from at least about 0.75 mg/kg up to not more than about 1.25 mg/kg; from at least about 0.95 mg/kg up to not more than about 1.05 mg/kg; and may be around about 1 mg/kg.

In some embodiments, an initial dose of a CD47 or SIRPα binding agent is infused over a period of at least about 2 hours, at least about 2.5 hours, at least about 3 hours, at least about 3.5 hours, at least about 4 hours, at least about 4.5 hours, at least about 5 hours, at least about 6 hours or more. In some embodiments an initial dose is infused over a period of time from about 2.5 hours to about 6 hours; for example, from about 3 hours to about 4 hours. In some such embodiments, the dose of agent in the infusate is from about 0.05 mg/ml to about 0.5 mg/ml; for example, from about 0.1 mg/ml to about 0.25 mg/ml.

In other embodiments, an initial dose of a CD47 or SIRPα binding agent, e.g., a priming dose, is administered by continuous fusion, e.g., as an osmotic pump, delivery patch, etc., where the dose is administered over a period of at least about 6 hours, at least about 12 hours, at least about 24 hours, at least about 2 days, at least about 3 days. Many such systems are known in the art. For example, DUROS technology, provides a bi-compartment system separated by a piston. One of the compartments consists of osmotic engine specifically formulated with an excess of solid NaCl, such that it remains present throughout the delivery period and results in a constant osmotic gradient. It also consists of a semi permeable membrane on one end through which water is drawn into the osmotic engine and establishes a large and constant osmotic gradient between the tissue water and the osmotic engine. Other compartment consists of a drug solution with an orifice from which the drug is released due to the osmotic gradient. This helps to provide site specific and systemic drug delivery when implanted in humans. The preferred site of implantation is subcutaneous placement in the inside of the upper arm.

Following administration of the priming agent, and allowing a period of time effective for an increase in reticulocyte production, a therapeutic dose of an anti-CD47 or anti-SIRPα agent is administered. The therapeutic dose can be administered in number of different ways. In some embodiments, two or more therapeutically effective doses are administered after a primer agent is administered, e.g., in a weekly dosing schedule. In some embodiments a therapeutically effective dose of an anti-CD47 agent is administered as two or more doses of escalating concentration, in others the doses are equivalent. There is reduced hemagglutination after the priming dose.

A therapeutically effective dose of an anti-SIRPα antibody can depend on the specific agent used, but is usually about 10 mg or more, e.g., about 30 mg, 50 mg, 100 mg, 200 mg, 400 mg or 800 mg, or more. Multiple administrations of an anti-SIRPα antibody, e.g., without Fc effector function, can be performed over an extended period of time, e.g., over 1, 2, 3, 4, 5, 6, 7, 8, 9, 10, 11, 12 months, at regular intervals, e.g., every 2 weeks (Q2W), every 3 weeks (Q3W), every 4 weeks (Q4W).

With respect to dosing of the NEDD8-activating enzyme E1 regulatory subunit (NAE1) inhibitor (e.g., pevonedistat), in some embodiments, the pevonedistat is administered at one or more doses in the range of 10 mg/m² to 50 mg/m², e.g., 10 mg/m², 15 mg/m², 20 mg/m², 25 mg/m², or 50 mg/m².

A therapeutically effective dose of a hypomethylating agent can be from 10 to 150 mg/kg. In some embodiments, the therapeutically effective dose of a hypomethylating agent is from 10-20, 20-30, 30-40, 40-50, 50-60, 60-70, 75, 70-80, 80-90, 90-100, 100-110, 110-120, 120-130, 130-140, or 140-150 mg/kg. In some embodiments, the therapeutically effective dose of a hypomethylating agent is about 10, 15, 20, 25, 30, 35, 40, 45, 50, 55, 60, 65, 70, 75, 80, 85, 90, 95, 100, 110, 120, 130, 140, or 150 mg/kg.

A therapeutically effective dose of azacitidine can be from 10 to 150 mg/kg. In some embodiments, the therapeutically effective dose of azacitidine is from 10-20, 20-30, 30-40, 40-50, 50-60, 60-70, 75, 70-80, 80-90, 90-100, 100-110, 110-120, 120-130, 130-140, or 140-150 mg/kg. In some embodiments, the therapeutically effective dose of azacitidine is about 10, 15, 20, 25, 30, 35, 40, 45, 50, 55, 60, 65, 70, 75, 80, 85, 90, 95, 100, 110, 120, 130, 140, or 150 mg/kg. In some embodiments, the therapeutically effective dose of azacitidine is 75 mg/kg. In some embodiments, the azacitidine is administered at a dose of at least 75 mg/m².

In some embodiments, the agent that inhibits binding between CD47 and SIRPα; and the NEDD8-activating enzyme E1 regulatory subunit (NAE1) inhibitor are administered in a combined synergistic amount. A “combined synergistic amount” as used herein refers to the sum of a first amount (e.g., an amount of an agent that inhibits binding between CD47 and SIRPα) and a second amount (e.g., an amount of an NAE1 inhibitor) that results in a synergistic effect (i.e., an effect greater than an additive effect). Therefore, the terms “synergy”, “synergism”, “synergistic”, “combined synergistic amount”, and “synergistic therapeutic effect” which are used herein interchangeably, refer to a measured effect of compounds administered in combination where the measured effect is greater than the sum of the individual effects of each of the compounds administered alone as a single agent.

Co-administration of an agent that inhibits binding between CD47 and SIRPα and a NAE1 inhibitor can allow for lower doses of one or both therapeutic agents. In embodiments, a synergistic amount may be about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the agent that inhibits binding between CD47 and SIRPα when used separately from the NAE1 inhibitor. In embodiments, a synergistic amount may be about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of NAE1 inhibitor when used separately from the agent that inhibits binding between CD47 and SIRPα.

Dosage and frequency may vary depending on the half-life of the therapeutic agent in the patient. It will be understood by one of skill in the art that such guidelines will be adjusted for the molecular weight of the active agent, e.g., in the use of antibody fragments, in the use of antibody conjugates, in the use of SIRPα reagents, in the use of soluble CD47 peptides etc. The dosage may also be varied for localized administration, e.g., intranasal, inhalation, etc., or for systemic administration, e.g., intramuscular (i.m.), intraperitoneal (i.p.), intravenous (i.v.), subcutaneous (s.c.), intratumoral, intracranial, and the like. In some embodiments, the agent that inhibits binding between CD47 and SIRPα; and the NAE1 inhibitor are administered concurrently. In some embodiments, the agent that inhibits binding between CD47 and SIRPα; and the NAE1 inhibitor are administered sequentially. For example, the agent that inhibits binding between CD47 and SIRPα, described herein, may be administered within seconds, minutes, hours or days of the administration of the NAE1 inhibitor. In some embodiments, a unit dose of an agent that inhibits binding between CD47 and SIRPα is administered first, followed within seconds, minutes, hours or days by administration of a unit dose of an NAE1 inhibitor. Alternatively, a unit dose of an NAE1 inhibitor is administered first, followed by administration of a unit dose of an agent that inhibits binding between CD47 and SIRPα within seconds, minutes, hours or days. In other embodiments, a unit dose of an agent that inhibits binding between CD47 and SIRPα is administered first, followed, after a period of hours (e.g., 1-12 hours, 1-24 hours, 1-36 hours, 1-48 hours, 1-60 hours, 1-72 hours), by administration of a unit dose of an NAE1 inhibitor. In yet other embodiments, a unit dose of an NAE1 inhibitor is administered first, followed, after a period of hours (e.g., 1-12 hours, 1-24 hours, 1-36 hours, 1-48 hours, 1-60 hours, 1-72 hours), by administration of a unit dose of an agent that inhibits binding between CD47 and SIRPα.

4. Conditions Subject to Treatment

Provided are methods of treating, ameliorating, mitigating, or preventing or delaying the recurrence or metastasis of, a cancer in a subject comprising administering: (a) an agent that inhibits binding between CD47 and SIRPα; and (b) a NEDD8-activating enzyme E1 regulatory subunit (NAE1) inhibitor to the subject.

As used herein, “treatment” or “treating” is an approach for obtaining beneficial or desired results including clinical results. For example, beneficial or desired clinical results may include one or more of the following: (i) decreasing one more symptoms resulting from the disease; (ii) diminishing the extent of the disease, stabilizing the disease (e.g., preventing or delaying the worsening of the disease); (iii) preventing or delaying the spread (e.g., metastasis) of the disease; (iv) preventing or delaying the occurrence or recurrence of the disease, delay or slowing the progression of the disease; (v) ameliorating the disease state, providing a remission (whether partial or total) of the disease, decreasing the dose of one or more other medications required to treat the disease; (vi) delaying the progression of the disease, increasing the quality of life, and/or (vii) prolonging survival. The beneficial or desired clinical results may be observed in more patients or subjects who have received the methods or treatments described herein.

“Prevention” or “preventing” means any treatment (i.e., medication, drug, therapeutic) of a disease or condition (i.e., cancer) that causes the clinical symptoms of the disease or condition not to develop. Compounds may, in some embodiments, be administered to a subject (including a human) who is at risk or has a family history of the disease or condition.

“Delaying” the development of a cancer means to defer, hinder, slow, retard, stabilize, and/or postpone development of the disease. The delay can be of varying lengths of time, depending on the history of the disease and/or subject being treated. As is evident to one of skill in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Disease development can be detectable using standard methods, such as routine physical exams, blood draw, mammography, imaging, or biopsy. Development may also refer to disease progression that may be initially undetectable and includes occurrence, recurrence, and onset.

The term “ameliorating” refers to any therapeutically beneficial result in the treatment of a disease state, e.g., a cancer disease state, including prophylaxis, lessening in the severity or progression, remission, or cure thereof.

In some embodiments, the subject has a hematopoietic disorder. Hematopoietic disorders include blood cancers, blood pre-cancers, blood disorders, blood dysplasia, blood hyperproliferative disorders, hematological cancers, hematologic malignancies, hematologic disorders, leukemias, pre-leukemias, acute myeloid leukemia (AML), myelodysplastic syndromes (MDS), clonal hematopoiesis (CH), clonal hematopoiesis of indeterminant potential (CHIP), age-related clonal hematopoiesis (ARCH), idiopathic cytopenias of undetermined significance (ICUS), and clonal cytopenia of undetermined significance (CCUS). A hematopoietic disorder can include a blood cancer or blood pre-cancer that includes one or more p53 mutations. A hematopoietic disorder can be a blood cancer. A hematopoietic disorder can be AML. A hematopoietic disorder can be MDS.

Selection and treatment of a subject having MDS with an anti-CD47 agent or an anti-SIRPα agent as described herein can be based on risk stratification of the subject. Cytogenetic abnormalities are seen in more than 80% of subjects with MDS and include translocations or aneuploidy (see Greenberg et al., Myelodysplastic Syndromes, Version 2.2017, NCCN Clinical Practice Guidelines in Oncology. J Natl Compr Canc Netw. 15(1):60-87, 2017, which is hereby incorporated by reference in its entirety). The International Prognostic Scoring System (IPSS) or revised IPSS (R-IPSS) are the most common MDS classification systems (see Dotson and Lebowicz. Myelodysplastic Syndrome. In: StatPearls [Internet]. Treasure Island (FL): StatPearls Publishing; 2020. Available from: www.ncbi.nlm.nih.gov/books/NBK534126/, which is hereby incorporated by reference in its entirety).

The IPSS can be used to classify the MDS risk level of a subject for treatment with an anti-CD47 agent or an anti-SIRPα agent as described herein. The IPSS stratifies patient risk based on the percentage of blasts in bone marrow, karyotype, and number of cell lineages with cytopenias. Karyotype with a good prognosis can include a normal karyotype, —Y, deletion 5q, or deletion 20q. Karyotype with a poor prognosis can include complex cytogenetics (e.g., greater than three abnormalities) or chromosome 7 abnormalities. All other karyotypes can be categorized as intermediate risk. Based on these findings, a score can be calculated to determine a risk score of low, intermediate-1, intermediate-2, or high risk. In some embodiments, a subject is classified as having low risk MDS. In some embodiments, a subject is classified as having intermediate-1 risk MDS. In some embodiments, a subject is classified as having intermediate-2 risk MDS. In some embodiments, a subject is classified as having high risk MDS.

The R-IPSS can be used to classify the MDS risk level of a subject for treatment with an anti-CD47 agent or an anti-SIRPα agent as described herein. The newer R-IPSS stratifies patient risk based on cytogenetics, blast percentage, and has separate scores for absolute neutrophil count, hemoglobin value, and platelet value. The R-IPSS can be used to stratify subjects into one of five categories: very good, good, intermediate, high, and very-high risk. In some embodiments, a subject is classified as having a very good prognosis of MDS. In some embodiments, a subject is classified as having a good prognosis of MDS. In some embodiments, a subject is classified as having an intermediate risk of MDS. In some embodiments, a subject is classified as having a high risk of MDS. In some embodiments, a subject is classified as having a very high risk of MDS.

In various embodiments, the subject has a B-cell hematologic malignancy, e.g., a CD20+ cancer, an indolent or aggressive lymphoma, e.g., diffuse large B-cell lymphoma (DLBCL) (including relapsed or refractory), follicular lymphoma (FL) (including relapsed, refractory, or asymptomatic), non-Hodgkin's lymphoma (NHL) (including relapsed or refractory), marginal zone lymphoma (e.g., extranodal marginal-zone lymphoma), mantle cell lymphoma (MCL) (including relapsed or refractory), chronic lymphocytic leukemia (CLL)/small lymphocytic leukemia (including relapsed or refractory), Waldenström's macroglobulinemia/lymphoplasmacytic lymphoma, primary mediastinal B-cell lymphoma, Burkitt's lymphoma, double hit lymphoma (e.g., high grade B cell lymphoma with MYC and one or both of BCL2 or BCL6 rearrangement), myc-rearranged lymphoma, B-cell lymphoma unclassified, B-cell acute lymphoblastic leukemia (ALL) (e.g., Philadelphia chromosome-negative acute lymphoblastic leukemia), or post-transplant lymphoproliferative disease (PTLD). In some embodiments, the subject has low Diffuse Large B-Cell Lymphoma (DLBCL), e.g., de novo or transformed DLBCL, or activated B cell (ABC), germinal center B cell (GCB), or non-germinal center B cell (non-GCB) DLBCL. In some embodiments, the subject has NHL, e.g., one or both of (i) low-grade or high-risk NHL or (ii) follicular (e.g., bulky, non-bulky, or advanced follicular) or nonfollicular NHL. In some embodiments, the subject has a relapsed or refractory form of a B-cell hematologic malignancy.

Provided herein are methods for treating individuals having a CD20+ cancer or reducing the size of such cancer in the subject, comprising administering: a therapeutically effective amount of an anti-CD47 antibody to the subject; and, optionally a therapeutically effective amount of at least one additional agent to the subject such as an anti-CD20 agent.

In some embodiments, a CD20+ cancer is a B cell cancer. In some embodiments, a subject has a B-cell hematologic malignancy. In some embodiments, a CD20+ cancer is an indolent or aggressive lymphoma. In some embodiments, the subject has a relapsed or refractory form of a B-cell cancer. B cell cancers can include Non-Hodgkin's lymphoma (NHL). In some embodiments, the NHL is low-grade or high-risk NHL. In some embodiments, the NHL is follicular (e.g., bulky, non-bulky, or advanced follicular) or nonfollicular NHL. NHL can include indolent lymphoma. Indolent lymphoma can include follicular lymphoma (FL). Indolent lymphoma can include marginal zone lymphoma. NHL can include diffuse large B cell lymphoma (DLBCL). NHL can further include DLBCL subtypes such as de novo DLBCL or transformed DLBCL. DLBCL can be from different cells of origin including activated B cell, germinal center B cell, and double hit lymphoma. A CD20+ cancer can include diffuse large B-cell lymphoma (DLBCL) (including relapsed or refractory), follicular lymphoma (FL) (including relapsed, refractory, or asymptomatic), non-Hodgkin's lymphoma (NHL) (including relapsed or refractory), marginal zone lymphoma (e.g., extranodal marginal-zone lymphoma), mantle cell lymphoma (MCL) (including relapsed or refractory), chronic lymphocytic leukemia (CLL)/small lymphocytic leukemia (including relapsed or refractory), Waldenström's macroglobulinemia/lymphoplasmacytic lymphoma, primary mediastinal B-cell lymphoma, Burkitt's lymphoma, double hit lymphoma (e.g., high grade B cell lymphoma with MYC and one or both of BCL2 or BCL6 rearrangement), myc-rearranged lymphoma, B-cell lymphoma unclassified, B-cell acute lymphoblastic leukemia (ALL) (e.g., Philadelphia chromosome-negative acute lymphoblastic leukemia), or post-transplant lymphoproliferative disease (PTLD). A given CD20+ cancer sub-type, such as those disclosed herein, can be classified based on histopathology, flow cytometry, molecular classification, one or more equivalent assays, or a combination thereof. A CD20+ cancer can include double hit lymphoma (e.g., high grade C cell lymphoma with MYC and BCL2 and/or BCL6 rearrangement). A CD20+ cancer can include a myc-rearranged lymphoma.

In some embodiments, the subject has a solid tumor. In various embodiments, the solid tumor arises from a primary malignancy having increased CD47 cell surface expression the surface, e.g., head and neck (HNSCC), melanoma, breast, lung, ovarian, pancreatic, colon, bladder, prostate, leiomyosarcoma, glioblastoma, medulloblastoma, oligodendroglioma, glioma, lymphoma, and multiple myeloma. In various embodiments, the cancer or tumor is malignant and/or a metastatic. In various embodiments, the subject has a cancer selected from the group consisting of an epithelial tumor (e.g., a carcinoma, a squamous cell carcinoma, a basal cell carcinoma, a squamous intraepithelial neoplasia), a glandular tumor (e.g., an adenocarcinoma, an adenoma, an adenomyoma), a mesenchymal or soft tissue tumor (e.g., a sarcoma, a rhabdomyosarcoma, a leiomyosarcoma, a liposarcoma, a fibrosarcoma, a dermatofibrosarcoma, a neurofibrosarcoma, a fibrous histiocytoma, an angiosarcoma, an angiomyxoma, a leiomyoma, a chondroma, a chondrosarcoma, an alveolar soft-part sarcoma, an epithelioid hemangioendothelioma, a Spitz tumor, a synovial sarcoma), and a lymphoma.

Examples of tissues containing cancerous cells whose proliferation is reduced or inhibited by combined administration of an agent that inhibits binding between CD47 and SIRPα (e.g., magrolimab); and a NEDD8-activating enzyme E1 regulatory subunit (NAE1) inhibitor (e.g., pevonedistat) include but are not limited to breast, prostate, brain, blood, bone marrow, liver, pancreas, skin, kidney, colon, ovary, lung, testicle, penis, thyroid, parathyroid, pituitary, thymus, retina, uvea, conjunctiva, spleen, head, neck, trachea, gall bladder, rectum, salivary gland, adrenal gland, throat, esophagus, lymph nodes, sweat glands, sebaceous glands, muscle, heart, and stomach.

In various embodiments, the subject has a solid tumor in or arising from a tissue or organ selected from the group consisting of:
bone (e.g., adamantinoma, aneurysmal bone cysts, angiosarcoma, chondroblastoma, chondroma, chondromyxoid fibroma, chondrosarcoma, chordoma, dedifferentiated chondrosarcoma, enchondroma, epithelioid hemangioendothelioma, fibrous dysplasia of the bone, giant cell tumour of bone, haemangiomas and related lesions, osteoblastoma, osteochondroma, osteosarcoma, osteoid osteoma, osteoma, periosteal chondroma, Desmoid tumor, Ewing sarcoma);
lips and oral cavity (e.g., odontogenic ameloblastoma, oral leukoplakia, oral squamous cell carcinoma, primary oral mucosal melanoma); salivary glands (e.g., pleomorphic salivary gland adenoma, salivary gland adenoid cystic carcinoma, salivary gland mucoepidermoid carcinoma, salivary gland Warthin's tumors);
esophagus (e.g., Barrett's esophagus, dysplasia and adenocarcinoma);
gastrointestinal tract, including stomach (e.g., gastric adenocarcinoma, primary gastric lymphoma, gastrointestinal stromal tumors (GISTs), metastatic deposits, gastric carcinoids, gastric sarcomas, neuroendocrine carcinoma, gastric primary squamous cell carcinoma, gastric adenoacanthomas), intestines and smooth muscle (e.g., intravenous leiomyomatosis), colon (e.g., colorectal adenocarcinoma), rectum, anus;
pancreas (e.g., serous neoplasms, including microcystic or macrocystic serous cystadenoma, solid serous cystadenoma, Von Hippel-Landau (VHL)-associated serous cystic neoplasm, serous cystadenocarcinoma; mucinous cystic neoplasms (MCN), intraductal papillary mucinous neoplasms (IPMN), intraductal oncocytic papillary neoplasms (IOPN), intraductal tubular neoplasms, cystic acinar neoplasms, including acinar cell cystadenoma, acinar cell cystadenocarcinoma, pancreatic adenocarcinoma, invasive pancreatic ductal adenocarcinomas, including tubular adenocarcinoma, adenosquamous carcinoma, colloid carcinoma, medullary carcinoma, hepatoid carcinoma, signet ring cell carcinoma, undifferentiated carcinoma, undifferentiated carcinoma with osteoclast-like giant cells, acinar cell carcinoma, neuroendocrine neoplasms, neuroendocrine microadenoma, neuroendocrine tumors (NET), neuroendocrine carcinoma (NEC), including small cell or large cell NEC, insulinoma, gastrinoma, glucagonoma, serotonin-producing NET, somatostatinoma, VIPoma, solid-pseudopapillary neoplasms (SPN), pancreatoblastoma);
gall bladder (e.g., carcinoma of the gallbladder and extrahepatic bile ducts, intrahepatic cholangiocarcinoma);
neuro-endocrine (e.g., adrenal cortical carcinoma, carcinoid tumors, phaeochromocytoma, pituitary adenomas);
thyroid (e.g., anaplastic (undifferentiated) carcinoma, medullary carcinoma, oncocytic tumors, papillary carcinoma, adenocarcinoma);
liver (e.g., adenoma, combined hepatocellular and cholangiocarcinoma, fibrolamellar carcinoma, hepatoblastoma, hepatocellular carcinoma, mesenchymal, nested stromal epithelial tumor, undifferentiated carcinoma; hepatocellular carcinoma, intrahepatic cholangiocarcinoma, bile duct cystadenocarcinoma, epithelioid hemangioendothelioma, angiosarcoma, embryonal sarcoma, rhabdomyosarcoma, solitary fibrous tumor, teratoma, York sac tumor, carcinosarcoma, rhabdoid tumor);
kidney (e.g., ALK-rearranged renal cell carcinoma, chromophobe renal cell carcinoma, clear cell renal cell carcinoma, clear cell sarcoma, metanephric adenoma, metanephric adenofibroma, mucinous tubular and spindle cell carcinoma, nephroma, nephroblastoma (Wilms tumor), papillary adenoma, papillary renal cell carcinoma, renal oncocytoma, renal cell carcinoma, succinate dehydrogenase-deficient renal cell carcinoma, collecting duct carcinoma);
breast (e.g., invasive ductal carcinoma, including without limitation, acinic cell carcinoma, adenoid cystic carcinoma, apocrine carcinoma, cribriform carcinoma, glycogen-rich/clear cell, inflammatory carcinoma, lipid-rich carcinoma, medullary carcinoma, metaplastic carcinoma, micropapillary carcinoma, mucinous carcinoma, neuroendocrine carcinoma, oncocytic carcinoma, papillary carcinoma, sebaceous carcinoma, secretory breast carcinoma, tubular carcinoma; lobular carcinoma, including without limitation, pleomorphic carcinoma, signet ring cell carcinoma);
peritoneum (e.g., mesothelioma; primary peritoneal cancer);
female sex organ tissues, including ovary (e.g., choriocarcinoma, epithelial tumors, germ cell tumors, sex cord-stromal tumors), Fallopian tubes (e.g., serous adenocarcinoma, mucinous adenocarcinoma, endometrioid adenocarcinoma, clear cell adenocarcinoma, transitional cell carcinoma, squamous cell carcinoma, undifferentiated carcinoma, mullerian tumors, adenosarcoma, leiomyosarcoma, teratoma, germ cell tumors, choriocarcinoma, trophoblastic tumors), uterus (e.g., carcinoma of the cervix, endometrial polyps, endometrial hyperplasia, intraepithelial carcinoma (EIC), endometrial carcinoma (e.g., endometrioid carcinoma, serous carcinoma, clear cell carcinoma, mucinous carcinoma, squamous cell carcinoma, transitional carcinoma, small cell carcinoma, undifferentiated carcinoma, mesenchymal neoplasia), leiomyoma (e.g., endometrial stromal nodule, leiomyosarcoma, endometrial stromal sarcoma (ESS), mesenchymal tumors), mixed epithelial and mesenchymal tumors (e.g., adenofibroma, carcinofibroma, adenosarcoma, carcinosarcoma (malignant mixed mesodermal sarcoma-MMMT)), endometrial stromal tumors, endometrial malignant mullerian mixed tumours, gestational trophoblastic tumors (partial hydatiform mole, complete hydatiform mole, invasive hydatiform mole, placental site tumour)), vulva, vagina;
male sex organ tissues, including prostate, testis (e.g., germ cell tumors, spermatocytic seminoma), penis;
bladder (e.g., squamous cell carcinoma, urothelial carcinoma, bladder urothelial carcinoma);
brain, (e.g., gliomas (e.g., astrocytomas, including non-infiltrating, low-grade, anaplastic, glioblastomas; oligodendrogliomas, ependymomas), meningiomas, gangliogliomas); schwannomas (neurilemmomas), craniopharyngiomas, chordomas, Non-Hodgkin lymphomas (NHLs), indolent non-Hodgkin's lymphoma (iNHL), refractory iNHL, pituitary tumors; eye (e.g., retinoma, retinoblastoma, ocular melanoma, posterior uveal melanoma, iris hamartoma);
head and neck (e.g., nasopharyngeal carcinoma, Endolymphatic Sac Tumor (ELST), epidermoid carcinoma, laryngeal cancers including squamous cell carcinoma (SCC) (e.g., glottic carcinoma, supraglottic carcinoma, subglottic carcinoma, transglottic carcinoma), carcinoma in situ, verrucous, spindle cell and basaloid SCC, undifferentiated carcinoma, laryngeal adenocarcinoma, adenoid cystic carcinoma, neuroendocrine carcinomas, laryngeal sarcoma), head and neck paragangliomas (e.g., carotid body, jugulotympanic, vagal);
thymus (e.g., thymoma);
heart (e.g., cardiac myxoma);
lung (e.g., small cell carcinoma (SCLC), non-small cell lung carcinoma (NSCLC), including squamous cell carcinoma (SCC), adenocarcinoma and large cell carcinoma, carcinoids (typical or atypical), carcinosarcomas, pulmonary blastomas, giant cell carcinomas, spindle cell carcinomas, pleuropulmonary blastoma);
lymph (e.g., lymphomas, including Hodgkin's lymphoma, non-Hodgkin's lymphoma (NHL), indolent non-Hodgkin's lymphoma (iNHL), refractory iNHL, Epstein-Barr virus (EBV)-associated lymphoproliferative diseases, including B cell lymphomas and T cell lymphomas (e.g., Burkitt lymphoma; large B cell lymphoma, diffuse large B-cell lymphoma (DLBCL), mantle cell lymphoma, indolent B-cell lymphoma, low grade B cell lymphoma, fibrin-associated diffuse large cell lymphoma; primary effusion lymphoma; plasmablastic lymphoma; extranodal NK/T cell lymphoma, nasal type; peripheral T cell lymphoma, cutaneous T cell lymphoma, angioimmunoblastic T cell lymphoma; follicular T cell lymphoma; systemic T cell lymphoma), lymphangioleiomyomatosis);
central nervous system (CNS) (e.g., gliomas including astrocytic tumors (e.g., pilocytic astrocytoma, pilomyxoid astrocytoma, subependymal giant cell astrocytoma, pleomorphic xanthoastrocytoma, diffuse astrocytoma, fibrillary astrocytoma, gemistocytic astrocytoma, protoplasmic astrocytoma, anaplastic astrocytoma, glioblastoma (e.g., giant cell glioblastoma, gliosarcoma, glioblastoma multiforme) and gliomatosis cerebri), oligodendroglial tumors (e.g., oligodendroglioma, anaplastic oligodendroglioma), oligoastrocytic tumors (e.g., oligoastrocytoma, anaplastic oligoastrocytoma), ependymal tumors (e.g., subependymom, myxopapillary ependymoma, ependymomas (e.g., cellular, papillary, clear cell, tanycytic), anaplastic ependymoma), optic nerve glioma, and non-gliomas (e.g., choroid plexus tumors, neuronal and mixed neuronal-glial tumors, pineal region tumors, embryonal tumors, medulloblastoma, meningeal tumors, primary CNS lymphomas, germ cell tumors, Pituitary adenomas, cranial and paraspinal nerve tumors, stellar region tumors); neurofibroma, meningioma, peripheral nerve sheath tumors, peripheral neuroblastic tumours (including without limitation neuroblastoma, ganglioneuroblastoma, ganglioneuroma), trisomy 19 ependymoma);
neuroendocrine tissues (e.g., paraganglionic system including adrenal medulla (pheochromocytomas) and extra-adrenal paraganglia ((extra-adrenal) paragangliomas);
skin (e.g., clear cell hidradenoma, cutaneous benign fibrous histiocytomas, cylindroma, hidradenoma, melanoma (including cutaneous melanoma, mucosal melanoma), basal cell carcinoma, pilomatricoma, Spitz tumors); and
soft tissues (e.g., aggressive angiomyxoma, alveolar rhabdomyosarcoma, alveolar soft part sarcoma, angiofibroma, angiomatoid fibrous histiocytoma, synovial sarcoma, biphasic synovial sarcoma, clear cell sarcoma, dermatofibrosarcoma protuberans, desmoid-type fibromatosis, small round cell tumor, desmoplastic small round cell tumor, elastofibroma, embryonal rhabdomyosarcoma, Ewing's tumors/primitive neurectodermal tumors (PNET), extraskeletal myxoid chondrosarcoma, extraskeletal osteosarcoma, paraspinal sarcoma, inflammatory myofibroblastic tumor, lipoblastoma, lipoma, chondroid lipoma, liposarcoma/malignant lipomatous tumors, liposarcoma, myxoid liposarcoma, fibromyxoid sarcoma, lymphangioleiomyoma, malignant myoepithelioma, malignant melanoma of soft parts, myoepithelial carcinoma, myoepithelioma, myxoinflammatory fibroblastic sarcoma, undifferentiated sarcoma, pericytoma, rhabdomyosarcoma, non-rhabdomyosarcoma soft tissue sarcoma (NRSTS), soft tissue leiomyosarcoma, undifferentiated sarcoma, well-differentiated liposarcoma.

5. Kits

Also described herein are kits comprising one or more unitary doses of the active agents, e.g., an agent that inhibits binding between CD47 and SIRPα (e.g., magrolimab) and a NEDD8-activating enzyme E1 regulatory subunit (NAE1) inhibitor (e.g., pevonedistat), and formulations thereof, as described herein, and instructions for use. In various embodiments, the agent that inhibits binding between CD47 and SIRPα and the NAE1 inhibitor can be in the same or different containers. The kit can further contain a least one additional reagent, e.g., a hypomethylation agent (e.g., azacitidine). Kits typically include a label indicating the intended use of the contents of the kit. The term label includes any writing, or recorded material supplied on or with the kit, or which otherwise accompanies the kit.

In some embodiments, one or both of the agent that inhibits binding between CD47 and SIRPα (e.g., magrolimab) and the NAE1 inhibitor (e.g., pevonedistat) are provided in a dosage form (e.g., a therapeutically effective dosage form). In some embodiments, one or both of the agent that inhibits binding between CD47 and SIRPα (e.g., magrolimab) and the NAE1 inhibitor (e.g., pevonedistat) are provided in two or more different dosage forms (e.g., two or more different therapeutically effective dosage forms). In the context of a kit, one or both of the agent that inhibits binding between CD47 and SIRPα (e.g., magrolimab) and the NAE1 inhibitor (e.g., pevonedistat) can be provided in liquid or sold form in any convenient packaging (e.g., stick pack, dose pack, etc.).

In various embodiments, the subject kits include a primer agent (e.g., an erythropoiesis-stimulating agent (ESA)) and an anti-CD47 agent. In some embodiments, a kit comprises two or more primer agents. In some embodiments, a kit comprises two or more anti-CD47 agents. In some embodiments, a primer agent is provided in a dosage form (e.g., a priming dosage form). In some embodiments, a primer agent is provided in two or more different dosage forms (e.g., two or more different priming dosage forms).

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### EXAMPLES

The following examples are offered to illustrate, but not to limit the claims.

### Example 1

### Combination of NAE1 SMI with Magrolimab Enhanced the Phagocytic Elimination of Acute Myelogenous Leukemia (AML) Cells

This study was designed to evaluate whether the combination of a NAE1 small molecule inhibitor (NAE1 SMI) with magrolimab could increase the elimination of leukemia cells in vitro and in vivo.

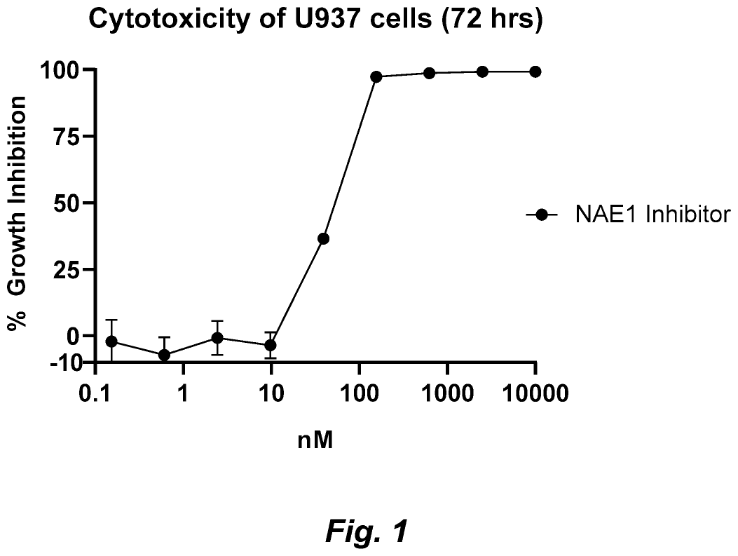

First, to confirm the therapeutic effect of the NAE1 SMI against leukemia cancer cells, human acute myelogenous leukemia (AML) cells (U937) were incubated with increasing doses of the NAE1 SMI. Consistent with the known effect of NAE1 SMI, a dosed-dependent growth inhibition of U937 AML cells by the NAE1 SMI was confirmed in the study (FIG. 1).

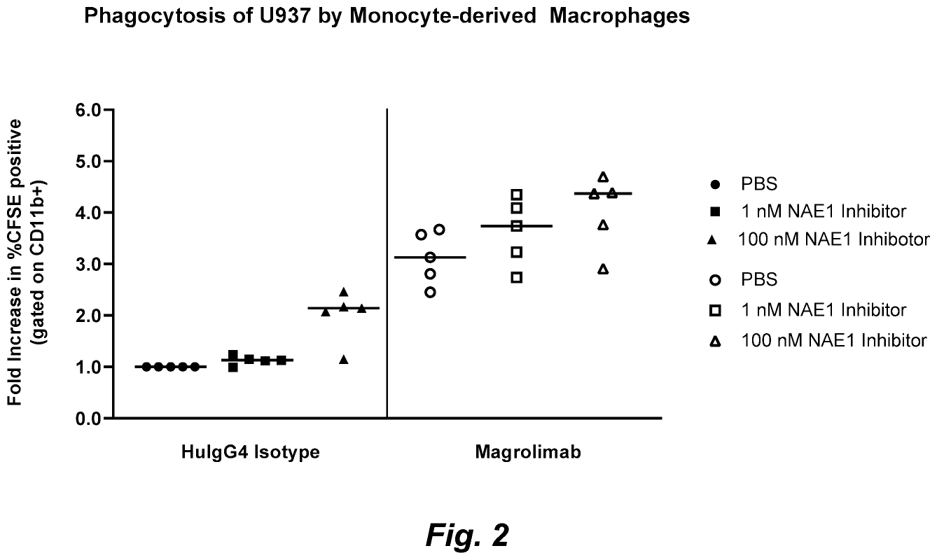

Next, to evaluate if the combination of the NAE1 SMI with magrolimab could increase the elimination of leukemia cells in vitro, a phagocytosis assay was performed. U937 AML cells with or without prior exposure to NAE1 SMI were incubated with macrophages derived from 5 human donors in the presence of magrolimab or IgG control. Consistent with the conclusion, the combination of magrolimab with NAE1 SMI enhanced the phagocytosis of the U937 AML cells compared to treatment with either agent alone, and in a dose-dependent manner (FIG. 2).

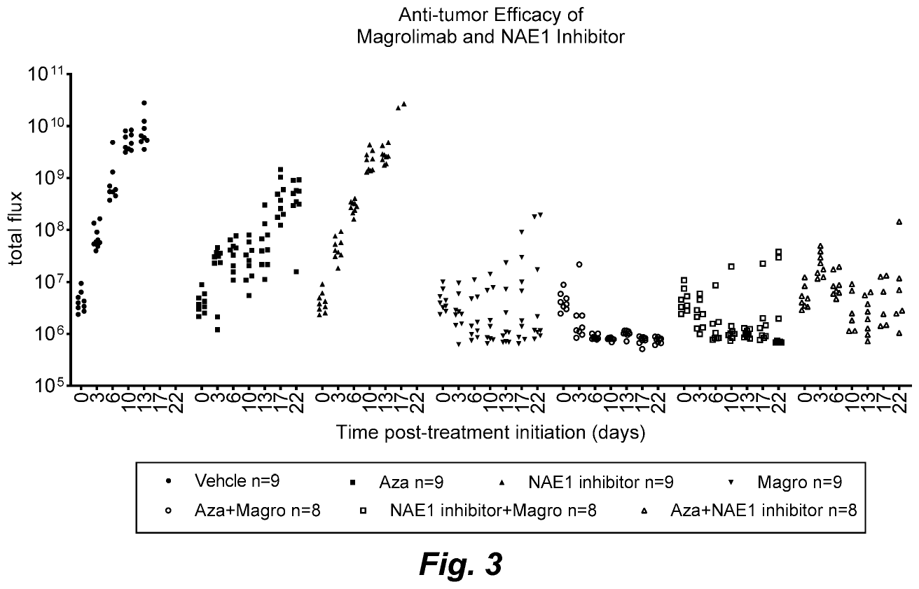

To evaluate, if the combination of NAE1 SMI with magrolimab could increase the elimination of leukemia cells in vivo, a treatment study was conducted in mice transplanted with U937 AML cells. In addition to NAE1 SMI, azacitidine, a hypomethylating and chemotherapeutic agent indicated for AML was also included into the study, since azacitidine had previously been established to enhance elimination of AML cells when combined with magrolimab (Chao, et al., Front Oncol (2020) 9:1380). After cancer cell transplantation and conformation of engraftment, the mice were randomized in 7 treatment cohorts: (1) vehicle control, (2) azacitidine, (3) NAE1 SMI, (4) magrolimab, (5) azacitidine+magrolimab, (6) NAE1 SMI+magrolimab, (7) azacitidine+NAE1 SMI. Consistent with the prior study (Chao, et al., supra), combination of azacytidine with magrolimab induced a durable cancer remission in all mice while treatment with azacytidine or magrolimab alone did initially slow tumor growth compared to vehicle control but failed to stop cancer progression. Consistent with the conclusion, the combination of NAE1 SMI with magrolimab did induce a durable cancer remission in the majority of the mice (6/8) and slowed cancer growth in the other mice (2/8) while NAE1 SMI alone did not achieve any inhibition of cancer cell growth. In addition, the combination of azacitidine with NAE1 SMI was more potent to slow cancer growth compared to single agent treatment with either molecule but failed to produce a durable cancer remission (FIG. 3).

In summary, the combination of NAE1 SMI with magrolimab enhanced the phagocytic elimination of AML cells by human macrophages in vitro, and enhanced clearance of AML cancer cells in vivo while single agent treatment with magrolimab or NAE1 SMI only achieved a modest or no inhibition of cancer growth, respectively.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating, mitigating, or preventing or delaying the progression of, or preventing or delaying the recurrence or metastasis of, a cancer in a subject comprising administering: (a) an agent that inhibits binding between CD47 and SIRPα; and (b) a NEDD8-activating enzyme E1 regulatory subunit (NAE1) inhibitor to the subject.

2. The method of claim 1, wherein the cancer is a hematologic cancer.

3. (canceled)

4. The method of claim 1, wherein the cancer has increased cell surface expression of CD47.

5. (canceled)

6. The method of claim 2, wherein the cancer is a leukemia or a pre-leukemia.

7. The method of claim 6, wherein the cancer is selected from the group consisting of a myelodysplastic syndrome (MDS), acute myelogenous leukemia (AML), chronic lymphocytic leukemia (CLL), small lymphocytic leukemia (SLL), B-cell acute lymphoblastic leukemia.

8. The method of claim 2, wherein the cancer is a lymphoma.

9. The method of claim 8, wherein the lymphoma is selected from the group consisting of non-Hodgkin's lymphoma, diffuse large B-cell lymphoma (DLBCL), follicular lymphoma (FL), marginal zone lymphoma, mantle cell lymphoma, Waldenström's macroglobulinemia/lymphoplasmacytic lymphoma, primary mediastinal B-cell lymphoma, Burkitt's lymphoma, B-cell lymphoma unclassified, or post-transplant lymphoproliferative disease (PTLD).

10. The method of claim 1, wherein the cancer is relapsed or refractory.

11. The method of claim 1, wherein the agent that inhibits binding between CD47 and SIRPα comprises an antibody that binds to CD47.

12. The method of claim 11, the antibody that binds to CD47 is selected from the group consisting of magrolimab, lemzoparlimab, letaplimab, AK117 (ligufalimab), AO-176, IBI-322, ZL-1201, IMC-002, SRF-231, CC-90002 (a.k.a., INBRX-103), NI-1701 (a.k.a., TG-1801) and STI-6643.

13. The method of claim 1, wherein the agent that inhibits binding between CD47 and SIRPα comprises an antibody that binds to SIRPα.

14. The method of claim 11, the antibody that binds to SIRPα is selected from the group consisting of GS-0189 (a.k.a., FSI-189), CC-95251, BI-765063 and APX-700.

15. The method of claim 1, wherein the agent that inhibits binding between CD47 and SIRPα comprises a SIRPα-Fc fusion protein.

16. The method of claim 15, the SIRPα-Fc fusion protein is selected from the group consisting of ALX-148 (evorpacept), TTI-621, TTI-622, JMT601 (CP0107) and SL-172154.

17. The method of claim 1, wherein the NAE1 inhibitor is selected from the group consisting of pevonedistat, TAK-243 and TAS-4464.

18. The method of claim 1, wherein the agent that inhibits binding between CD47 and SIRPα and the NAE1 inhibitor are administered concurrently.

19. The method of claim 1, wherein the agent that inhibits binding between CD47 and SIRPα and the NAE1 inhibitor are administered sequentially.

20-25. (canceled)

26. The method of claim 1, wherein the agent that inhibits binding between CD47 and SIRPα; and the NAE1 are administered in a combined synergistic amount.

27. The method of claim 1, wherein administration of the agent that inhibits binding between CD47 and SIRPα and the NAE1 inhibitor provides a synergistic effect.

28. The method of claim 27, wherein the synergistic effect is increased cancer cell death and/or decreased cancer cell growth when comparing the effect of the combination versus either the agent that inhibits binding between CD47 and SIRPα or the NAE1 inhibitor alone.

29. The method of claim 27, wherein the synergistic effect is increased phagocytosis of cancer cells by macrophages when comparing the effect of the combination versus either the agent that inhibits binding between CD47 and SIRPα or the NAE1 inhibitor alone.

30. The method of claim 27, wherein the synergistic effect is increased or enhanced cancer cell clearance when comparing the effect of the combination versus either the agent that inhibits binding between CD47 and SIRPα or the NAE1 inhibitor alone.

31. A method of treating, mitigating, or preventing or delaying the recurrence or metastasis of, a cancer in a subject comprising administering: (a) magrolimab; and (b) pevonedistat to the subject.

32. The method of claim 31, wherein the cancer is a hematologic cancer.

33. (canceled)

34. The method of claim 32, wherein the cancer has increased cell surface expression of CD47.

35. (canceled)

36. The method of claim 32, wherein the cancer is a leukemia or a pre-leukemia.

37. The method of claim 36, wherein the cancer is selected from the group consisting of a myelodysplastic syndrome (MDS), acute myelogenous leukemia (AML), chronic lymphocytic leukemia (CLL), small lymphocytic leukemia (SLL), B-cell acute lymphoblastic leukemia.

38. The method of claim 32, wherein the cancer is a lymphoma.

39. The method of claim 38, wherein the lymphoma is selected from the group consisting of non-Hodgkin's lymphoma, diffuse large B-cell lymphoma (DLBCL), follicular lymphoma (FL), marginal zone lymphoma, mantle cell lymphoma, Waldenström's macroglobulinemia/lymphoplasmacytic lymphoma, primary mediastinal B-cell lymphoma, Burkitt's lymphoma, B-cell lymphoma unclassified, or post-transplant lymphoproliferative disease (PTLD).

40. The method of claim 31, wherein the cancer is relapsed or refractory.

41-47. (canceled)

48. The method of claim 31, wherein administration of magrolimab and pevonedistat provides a synergistic effect.

49. The method of claim 48, wherein the synergistic effect is increased cancer cell death and/or decreased cancer cell growth when comparing the effect of the combination versus either magrolimab or pevonedistat alone.

50. The method of claim 48, wherein the synergistic effect is increased phagocytosis of cancer cells by macrophages when comparing the effect of the combination versus either magrolimab or pevonedistat alone.

51. The method of claim 48, wherein the synergistic effect is increased or enhanced cancer cell clearance when comparing the effect of the combination versus either the magrolimab or pevonedistat alone.

52. The method of claim 31, wherein the magrolimab is first administered at a priming dose of less than 10 mg/kg and then administered at one or more therapeutic doses of at least 15 mg/kg.

53. The method of claim 31, wherein the magrolimab is administered intravenously, subcutaneously or intratumorally.

54. The method of claim 31, wherein the pevonedistat is administered at one or more doses in the range of 10 mg/m² to 50 mg/m².

55. The method of claim 31, wherein the pevonedistat is administered orally, intravenously, intramuscularly or subcutaneously.

56. The method of claim 31, wherein the subject is a human.

57. A kit comprising one or more unitary doses of: (a) an agent that inhibits binding between CD47 and SIRPα; and (b) a NEDD8-activating enzyme E1 regulatory subunit (NAE1) inhibitor.

58-72. (canceled)
